(19) United States
(12) Patent Application Publication
(10) Pub. No.: US 2025/0066300 A1
(43) Pub. Date: Feb. 27, 2025

(54) 3-(2-(DIMETHYLAMINO)ETHYL)-1H-INDOL-4-yl OLIGOMERIC DERIVATIVES

(71) Applicant: Compass Pathfinder Limited, Altrincham (GB)

(72) Inventors: John Richard MORPHY, Altrincham (GB); Thomas Matthew BAKER, Nottingham (GB); Andrew William BROWN, Nottingham (GB)

(21) Appl. No.: 18/813,128

(22) Filed: Aug. 23, 2024

Related U.S. Application Data
(60) Provisional application No. 63/578,799, filed on Aug. 25, 2023.

(51) Int. Cl. C07D 209/16; A61K 31/4045

(52) U.S. Cl. CPC C07D 209/16; A61K 31/4045

(57) ABSTRACT

Provided herein are compounds of Formula (I), Formula (II), Formula (III), a compound selected from any of the compounds in Table 1, Table 2, Table 3, Table 4 or a pharmaceutically acceptable salt or deuterated form thereof, wherein R¹, R², R³, R⁴, R^{D}, R^{G}, p and q are defined herein. Also provided herein are pharmaceutical compositions comprising a compound of formula (I), or a compound selected from any of the compounds in Table 1, Table 2, Table 3, Table 4 or pharmaceutically acceptable salt or deuterated form thereof, and methods of using a compound of formula (I), Formula (II), Formula (III), or pharmaceutically acceptable salt or deuterated form thereof, e.g., in treating 5-HT₂A receptor associated diseases or disorders.

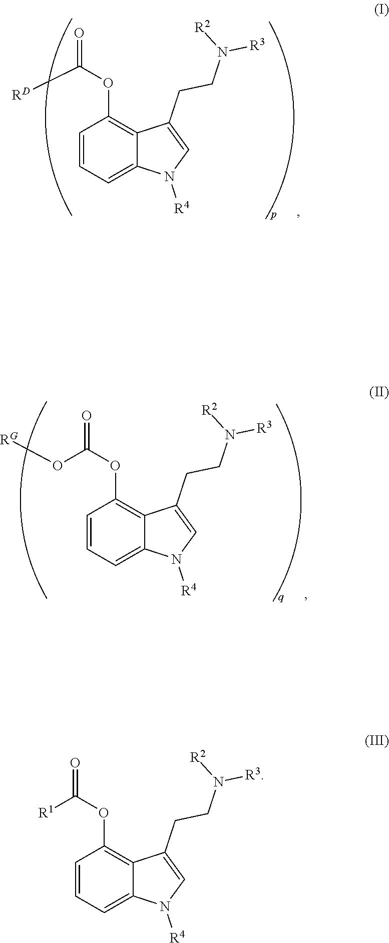

### CROSS-REFERENCE TO RELATED APPLICATIONS

This application claims priority to U.S. Provisional Application No. 63/578,799, filed Aug. 25, 2023, the disclosure of which is incorporated by reference in its entirety for all purposes.

### BACKGROUND

Over 50% of adults in the United States will be diagnosed with a psychiatric disorder at some point in their lifetime. Nearly 1 in 5 suffer from mental illness, and nearly 1 in 25 are afflicted with severe mental illness, such as major depression, schizophrenia, or bipolar disorder.

Psychedelics show promising activity in treating mental illness. New psychedelics compounds are needed for treating mental illness.

### BRIEF DESCRIPTION OF THE DRAWINGS

FIG. 1 shows pharmacokinetic data of compound 122 through subcutaneous administration.

### BRIEF DESCRIPTION

In some embodiments, the present disclosure provides a compound of formula (I):

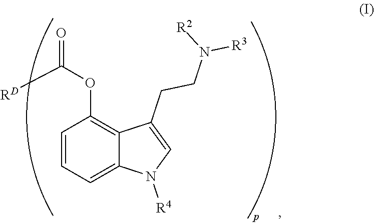

or a pharmaceutically acceptable salt thereof or deuterated form thereof, wherein:
   R² and R³ are independently alkyl;
   R⁴ is H or C(═O)Oalkyl;
   p is 2 or 3;
   R^{D} is a divalent or trivalent group selected from alkylene, alkenylene, alkylene-O-alkylene, —(CH₂)ₙ—(OCH₂CH₂)ₘ—, cycloalkylene, or arylene, or

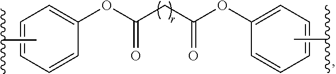

each of which is optionally substituted with 1-4 groups selected from halogen, OH, Oalkyl, alkyl, NH₂, NH(alkyl), N(alkyl)₂, C(═O)OH, C(═O)Oalkyl, OC(═O)alkyl, C(═O)alkyl or cycloalkyl;
n is 1, 2, 3, 4, 5, 6, 7 or 8;
m is 1, 2, 3, 4, 5, 6, 7 or 8;
r is 1, 2 or 3;
provided that when R² and R³ are each alkyl, then R^{D} is not CH₂, (CH₂)₂, (CH₂)₃, (CH₂)₄, (CH₂)₅, (CH₂)₈,

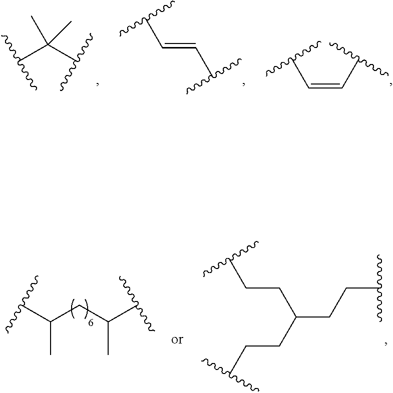

and provided that when R² and R³ are each deuterated alkyl then R^{D} is not (CH₂)₃.

In some embodiments, the present disclosure provides a compound of formula (II):

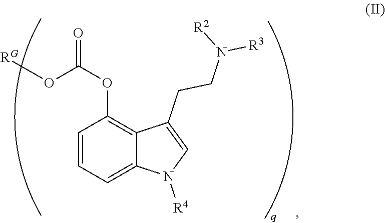

or a pharmaceutically acceptable salt or deuterated form thereof,
wherein:
   R² and R³ are independently alkyl;
   R⁴ is H or C(═O)Oalkyl;
   q is 2, 3 or 4;
   R^{G} is —(CH₂)ₙ—(OCH₂CH₂)ₘ—, or a divalent, trivalent or tetravalent alkylene or alkenylene;
   n is 1, 2, 3, 4, 5, 6, 7 or 8; and
   m is 0, 1, 2 3, 4, 5, 6, 7 or 8;
   provided that when q is 2 and R² and R³ are each CH₃, R^{G} is not —(CH₂)₂— or —(CH₂)₃—.

In some embodiments, the present disclosure provides a compound of formula (III):

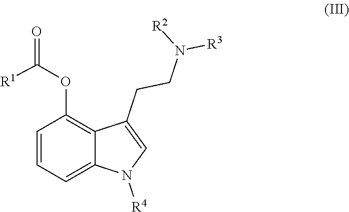

or a pharmaceutically acceptable salt thereof or deuterated form thereof, wherein:
   R² and R³ are independently alkyl;
   R⁴ is H or C(═O)Oalkyl;
   R¹ is alkylene-C(═O)OH wherein the alkylene is optionally substituted with OH, C(═O)OH, —OC(═O)alkyl or NH₂, alkenylene-C(═O)OH wherein the alkenylene is optionally substituted with C(═O)OH, cycloalkylene-C(═O)OH wherein the cycloalkylene is optionally substituted with C(═O)OH, arylene-C(═O)OH wherein the arylene is optionally substituted with alkyl, Oalkyl or C(═O)OH, —(CH₂CH₂O)ₘ—(CH₂CH₂), —C(═O)OH, alkylene-O-alkylene-C(═O)OH, arylene-O(C═O)-alkylene-C(═O)O-arylene-C(═O)OH, O-alkylene-OH wherein the alkylene is optionally substituted with OH, or —(OCH₂CH₂)ₘ—OH;
   m is 1, 2, 3,
   n is 1, 2, 3,
   provided that when provided that when R² and R³ are each alkyl, then R¹ is not (CH₂)₂, (CH₂)₃, —CH═CH—,

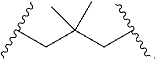 [SMILES: *CC(C)(C)C*]

### DETAILED DESCRIPTION

### Definitions

The terms below, as used herein, have the following meanings, unless indicated otherwise:

“Cyano” refers to the —CN radical.

“Hydroxy” or “hydroxyl” refers to the —OH radical.

“Oxo” refers to the ═O substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched multivalent (e.g., divalent, trivalent or tetravalent) hydrocarbon chain radical, and having from one to twelve carbon atoms. Non-limiting examples of C₁-C₁₂ alkylene include methylene, ethylene, propylene, n-butylene, and the like. As a non-limiting example, an alkylene chain is attached to the rest of the molecule through a single bond and to the radical group through a single bond. The points of attachment of the alkylene chain to the rest of the molecule and to the radical group can be through one carbon or any two or more carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkenyl group can be optionally substituted.

“Alkenylene” or “alkenylene chain” refers to a straight or branched multivalent (e.g., divalent, trivalent or tetravalent) hydrocarbon chain radical, having from two to twelve carbon atoms, and having one or more carbon-carbon double bonds. Non-limiting examples of C₂-C₁₂ alkenylene include ethenylene, propenylene, butenylene, and the like. As a non-limiting example, an alkenylene chain is attached to the rest of the molecule through a single bond and to the radical group through a single bond. The points of attachment of the alkenylene chain to the rest of the molecule and to the radical group can be through one carbon or any two or more carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkynyl group can be optionally substituted.

“Alkynylene” or “alkynylene chain” refers to a straight or branched multivalent (e.g., divalent, trivalent or tetravalent) hydrocarbon chain radical, having from two to twelve carbon atoms, and having one or more carbon-carbon triple bonds. Non-limiting examples of C₂-C₁₂ alkynylene include ethynylene, propargylene and the like. The points of attachment of the alkynylene chain to the rest of the molecule and to the radical group can be through one carbon or any two or more carbons within the chain. Unless stated otherwise specifically in the specification, an alkynylene chain can be optionally substituted.

“Alkoxy” refers to a radical of the formula —ORₐ where Rₐ is an alkyl, alkenyl or alkynyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Arylene” refers to a multivalent (e.g., divalent, trivalent or tetravalent) hydrocarbon ring system comprising hydrogen, 6 to 18 carbon atoms and at least one aromatic ring, and which is attached to the rest of the molecule by two or more single bonds. For purposes of this disclosure, the aryl can be a monocyclic, bicyclic, tricyclic or tetracyclic ring system, which can include fused or bridged ring systems. Arylenes include, but are not limited to, arylenes derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “arylene” can be optionally substituted.

“Aralkyl”, “arylalkyl” or “alkyene-aryl” refers to a radical of the formula —R_{b}—R_{c} where R_{b} is an alkylene group as defined above and R_{c} is one or more aryl radicals as defined above, for example, benzyl, diphenylmethyl and the like. Unless stated otherwise specifically in the specification, an aralkyl group can be optionally substituted.

“Carbocyclyl,” “carbocyclic ring” or “carbocycle” refers to a non-aromatic ring structure, wherein the atoms which form the ring are each carbon. Carbocyclic rings can comprise from 3 to 20 carbon atoms in the ring. Carbocyclic rings include cycloalkyl, cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, e.g., having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkene” refers to a multivalent (e.g., divalent, trivalent or tetravalent) non-aromatic monocyclic or polycyclic fully saturated hydrocarbon ring consisting solely of carbon and hydrogen atoms, which can include fused, spiro, or bridged ring systems, having from three to twenty carbon atoms, e.g., having from three to ten carbon atoms, and which is attached to the rest of the molecule by one or more single bonds. Monocyclic cycloalkylene include, for example, cyclopropylene, cyclobutylene, cyclopentylene, cyclohexylene, cycloheptylene, and cyclooctylene. Polycyclic cycloalkylene includes, for example, bicyclo[2.2.2]octanylene, cubanylene, bicyclo(1.1.1)pentylene, adamantylene, norbornylene, decalinylene, 7,7-dimethyl-bicyclo[2.2.1]heptanylene, and the like. Unless otherwise stated specifically in the specification, a cycloalkylene group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, e.g., having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, e.g., having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkynyl group can be optionally substituted.

“Heterocyclyl” “heterocyclic ring” or “heterocycle” refers to a stable 3- to 20-membered non-aromatic radical which consists of two to twelve carbon atoms and from one to six heteroatoms selected from nitrogen, oxygen, sulfur, or silicon. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heterocyclylene” refers to a multivalent (e.g., divalent, trivalent or tetravalent) 3- to 20-membered non-aromatic, partially aromatic, or aromatic ring radical which consists of two to twelve carbon atoms and from one to six heteroatoms selected from nitrogen, oxygen or sulfur. Unless stated otherwise specifically in the specification, the heterocyclylene radical can be a monocyclic, bicyclic, tricyclic or tetracyclic ring system, which can include fused, spiro, or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclylene radicals include, but are not limited to, dioxolanylene, decahydroisoquinolylene, imidazolinylene, imidazolidinylene, isothiazolidinylene, isoxazolidinylene, morpholinylene, octahydroindolylene, octahydroisoindolylene, 2-oxopiperazinylene, 2-oxopiperidinylene, 2-oxopyrrolidinylene, oxazolidinylene, piperidinylene, piperazinylene, 4-piperidonylene, pyrrolidinylene, pyrazolidinylene, quinuclidinylene, thiazolidinylene, tetrahydrofurylene, trithianylene, tetrahydropyranylene, thiomorpholinylene, thiamorpholinylene, 1-oxo-thiomorpholinylene, and 1,1-dioxo-thiomorpholinylene. Unless stated otherwise specifically in the specification, a heterocyclylene group can be optionally substituted.

“Heterocyclylalkyl” or “alkylene-heterocyclyl” refers to a radical of the formula —R_{b}—R_{c} where R_{b} is an alkylene group as defined above and Rₑ is a heterocyclyl radical as defined above. Unless stated otherwise specifically in the specification, a heterocycloalkyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heteroaryl” refers to a 5- to 20-membered ring system radical comprising hydrogen atoms, one to thirteen carbon atoms, one to six heteroatoms selected from nitrogen, oxygen and sulfur, and at least one aromatic ring comprising at least one heteroatom selected from nitrogen, oxygen and sulfur. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophene), benzotriazolyl, benzo[4,6]imidazo[1,2-a]pyridinyl, carbazolyl, cinnolinyl, dibenzofuranyl, dibenzothiophene, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophene (i.e. thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” or “alkylene-heteroaryl” refers to a radical of the formula —R_{b}—R_{f} where R_{b} is an alkylene chain as defined above and R_{f} is a heteroaryl radical as defined above. Unless stated otherwise specifically in the specification, a heteroarylalkyl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (e.g., alkyl, alkylene, alkenyl, alkenylene, alkynyl, alkynylene, alkoxy, alkylamino, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups.

“Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl.

“Substituted” further includes any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group.

In this specification, unless stated otherwise, the term “pharmaceutically acceptable” is used to characterize a moiety (e.g., a salt, dosage form, or excipient) as being appropriate for therapeutic use. In general, a pharmaceutically acceptable moiety has one or more benefits that outweigh any deleterious effect that the moiety may have. Deleterious effects may include, for example, excessive toxicity, irritation, allergic response, and other problems and complications.

The term “pharmaceutically acceptable salt” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The compounds of the disclosure, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

In some embodiments, “substituted” further means any alkyl, cycloalkyl or heterocyclylalkyl in which one or more hydrogen atoms is replaced by an isotope e.g., deuterium. In addition, each of the foregoing substituents can also be optionally substituted with one or more of the above substituents.

The compounds of the present disclosure may also contain unnatural proportions of atomic isotopes at one or more of the atoms that constitute such compounds. For example, the compounds may be radiolabeled with radioactive isotopes, such as for example deuterium (²H), tritium (³H), iodine-125 (¹²⁵I) or carbon-14 (¹⁴C). All isotopic variations of the compounds of the present disclosure, whether radioactive or not, are intended to be encompassed within the scope of the disclosure.

In some examples, unless otherwise specified, “rt” or “r.t.” means room temperature, “h” or “h.” means hour, “min” or “min.” means minutes, and “eq.” means equivalents.

### Compounds

The disclosure provides dimers, trimers, and tetramers of psilocin. The disclosed compounds are designed to release psilocin after administration to a subject in need thereof. In embodiments, the disclosed compounds have a psilocin release efficacy (as described in Example 14) ranging from about 7% to about 100%, including about 25% to about 100%, about 50% to about 100%, about 75% to about 100%, about 80% to about 100%, about 85% to about 100%, about 90% to about 100% or about 95% to about 100%. In embodiments, the disclosed compounds have a release efficacy of about 80% to about 100%. In some embodiments, this disclosure provides a compound of formula (I),

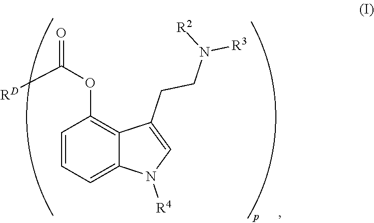

or a pharmaceutically acceptable salt thereof or deuterated form thereof, wherein:
   R² and R³ are independently alkyl;
   R⁴ is H or C(═O)Oalkyl;
   p is 2 (dimer), 3 (trimer), or 4 (tetramer);
   R^{D} is a divalent, trivalent, or tetravalent group selected from alkylene, alkenylene, alkylene-O-alkylene, —(CH₂)ₙ—(OCH₂CH₂)ₘ—, cycloalkylene, heterocyclylene, arylene, heteroarylene or

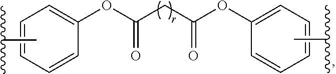

each of which is optionally substituted with OH, Oalkyl, alkyl, NH₂, NH(alkyl), N(alkyl)₂, C(═O)OH, C(═O)Oalkyl, OC(═O)alkyl, cycloalkyl, or C(═O)alkyl;
n is 1, 2, 3, 4, 5, 6, 7 or 8;
m is 1, 2, 3, 4, 5, 6, 7 or 8; or
r is 1, 2 or 3.

In some embodiments of formula (I), when R² and R³ are each non-deuterated alkyl, then R^{D} is not CH₂, (CH₂)₂, (CH₂)₃, (CH₂)₄, (CH₂)₅, (CH₂)₈,

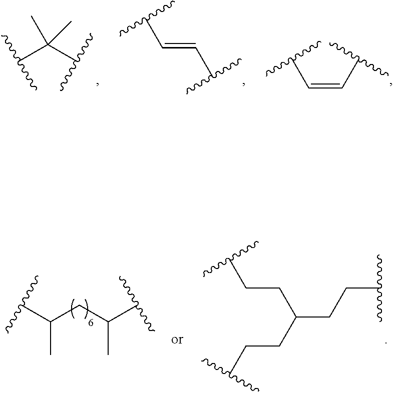

In some embodiments of formula (I), when R² and R³ are each deuterated alkyl, then R^{D} is not (CH₂)₃.

In some embodiments of formula (I), when R² and R³ are each CD₃, then R^{D} is not (CH₂)₃.

R^{D} can be any suitable moiety on to which 2, 3 or 4 psilocin residues can be conjugated to form psilocin dimers, trimers or tetramers. In some embodiments, R^{D} is a divalent or trivalent group selected from alkylene, alkylene-O-alkylene, —(CH₂)ₙ—(OCH₂CH₂)ₘ—, cycloalkylene, heterocyclylene, arylene, heteroarylene or

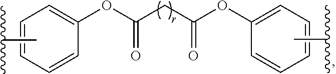

each of which is optionally substituted with OH, OCH₃, NH₂, COOH, cycloalkyl, or C(═O)alkyl. In some embodiments, R^{D} is a divalent or trivalent group selected from alkylene-O-alkylene, —(CH₂)ₙ—(OCH₂CH₂)ₘ—, cycloalkylene, heterocyclylene, arylene, heteroarylene or

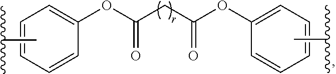

each of which is optionally substituted with OH, OCH₃, NH₂, COOH, cycloalkyl, or C(═O)alkyl. In some embodiments, R^{D} is a divalent alkylene. In some embodiments, R^{D} is a trivalent alkylene. In some embodiments, R^{D} is divalent alkylene-O-alkylene. In some embodiments, R^{D} is —(CH₂)ₙ—(OCH₂CH₂)ₘ— wherein n is 1-8 and m is 1-8. In some embodiments, R^{D} is divalent cycloalkylene. In some embodiments, R^{D} is trivalent cycloalkylene. In some embodiments, R^{D} is divalent arylene. In some embodiments, R^{D} is trivalent arylene. In some embodiments, R^{D} is

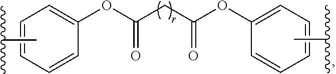

wherein r is 1-3. In some embodiments, R^{D} is optionally substituted with OH, OCH₃, NH₂, COOH, cycloalkyl, or C(═O)alkyl.

In some embodiments when the compound of formula (I) is deuterated, the compound has a structure of formula (D-I)

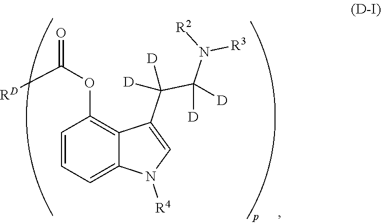

or a pharmaceutically acceptable salt thereof, wherein R^{D}, R², R³ and R⁴ are as defined in formula (I).

In some embodiments, R^{D} is R^{E}, and R^{E} is a divalent group. In some embodiments, the compound of formula (I) is a compound of formula (Ia)

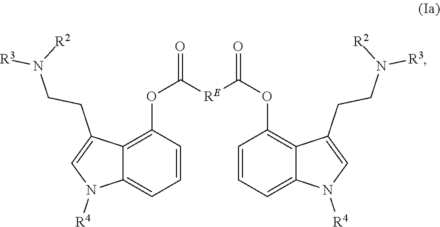

or a pharmaceutically acceptable salt or deuterated thereof,
   wherein:
each R² and R³ are independently alkyl;
each R⁴ is independently H or C(═O)Oalkyl;
R^{E} is a divalent group selected from alkylene, cycloalkylene, arylene, alkylene-O-alkylene, —(CH₂)ₙ—(OCH₂CH₂)ₘ— or

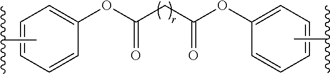

each of which is optionally substituted with one or more groups selected from Oalkyl, NH₂, NHC₁₋₆alkyl, N(C₁₋₆alkyl)₂, COOH, cycloalkyl, and C(═O)Oalkyl;
n is 1, 2, 3, 4, 5, 6, 7 or 8;
m is 1, 2 or 3; and
r is 1, 2 or 3.

In some embodiments, R^{E} is a divalent alkylene. In some embodiments, the alkylene group is linear or branched. In some embodiments, the alkylene group is linear. In some embodiments, the linear alkylene group is a C₃-C₇ alkylene. In some embodiments, the linear alkylene is substituted with —NH₂, OH, C(O)OH. In some embodiments, the alkylene group is branched. In some embodiments, the branched alkylene group is a C₃-C₅ alkylene. In some embodiments, the branched alkylene group is a C₃-C₅ alkylene is substituted with —NH₂, OH, C(O)OH.

In some embodiments, R^{E} is a divalent group selected from cycloalkylene, arylene, alkylene-O-alkylene, —(CH₂)ₙ—(OCH₂CH₂)ₘ— or

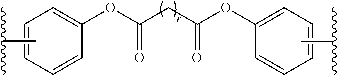

each of which is optionally substituted with one or more groups selected from Oalkyl, NH₂, NHC₁₋₆alkyl, N(C₁₋₆alkyl)₂, COOH, cycloalkyl, and C(═O)Oalkyl. In some embodiments, R^{E} is a divalent cycloalkylene. In some embodiments, R^{E} is divalent C₃-C₈ cycloalkylene. In some embodiments, R^{E} is divalent C₄ cycloalkylene. In some embodiments, R^{E} is divalent C₆ cycloalkylene. In some embodiments, R^{E} is

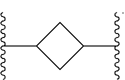 [SMILES: *C1CC(*)C1]

In some embodiments, R^{E} is

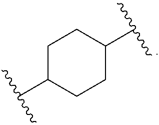 [SMILES: *C1CCC(*)CC1]

In some embodiments, R^{E} is divalent arylene. In some embodiments, R^{E} is divalent phenylene. In embodiments, the phenylene is substituted with Oalkyl, alkyl, or C(═O)OH. In embodiments, the divalent phenylene is substituted with OCH₃, CH₃ or C(═O)OH. In some embodiments, R^{E} is

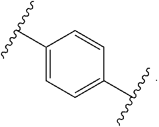 [SMILES: *c1ccc(*)cc1]

In some embodiments, R^{D} or R^{E} is

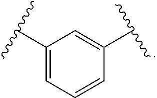 [SMILES: *c1cccc(*)c1]

In some embodiments, R^{D} or R^{E} is

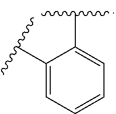 [SMILES: *c1ccccc1*]

In some embodiments, R^{D} or R^{E} is

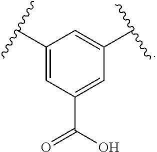 [SMILES: *c1cc(*)cc(C(=O)O)c1]

In some embodiments, R^{D} or R^{E} is

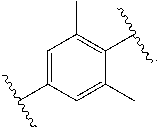 [SMILES: *c1cc(C)c(*)c(C)c1]

In some embodiments, R^{D} or R^{E} is

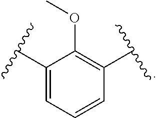 [SMILES: *c1cccc(*)c1OC]

In some embodiments, R^{E} is divalent alkylene-O-alkylene. In some embodiments, R^{E} is

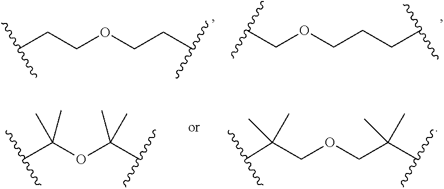

In some embodiments, R^{E} is

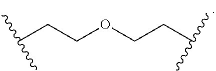 [SMILES: *CCOCC*]

In some embodiments, R^{E} is

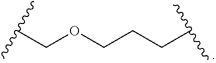 [SMILES: *CCCOC*]

In some embodiments, R^{E} is

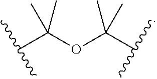 [SMILES: *C(C)(C)OC(*)(C)C]

In some embodiments, R^{E} is

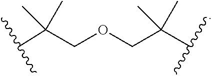 [SMILES: *C(C)(C)COCC(*)(C)C]

In some embodiments, R^{E} is —(CH₂)ₙ—(OCH₂CH₂)ₘ—. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, m is 1. In some embodiments, m is 2. In some embodiments, m is 3. In some embodiments, n is 1-3 and m is 1-4. In some embodiments, n is 2 and m is 2 or 3. In some embodiments, R^{E} is —(CH₂)₂—(OCH₂CH₂)₂—. In some embodiments, R^{E} is —(CH₂)₂—(OCH₂CH₂)₃—.

In some embodiments, R^{E}

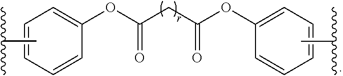

In some embodiments, r is 1. In some embodiments, r is 2. In some embodiments, r is 3. In some embodiments, R^{E} is

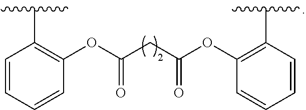

In some embodiments, R^{D} or R^{E} is (CH₂)₆, (CH₂)₇,

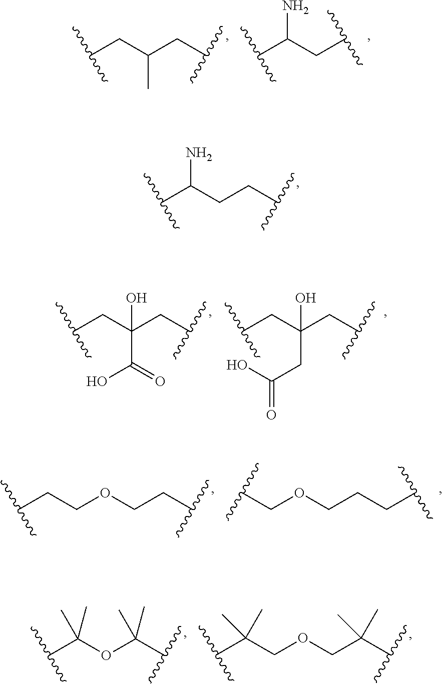

—(CH₂)₂—(OCH₂CH₂)₂—, —(CH₂)₂—(OCH₂CH₂)₃—,

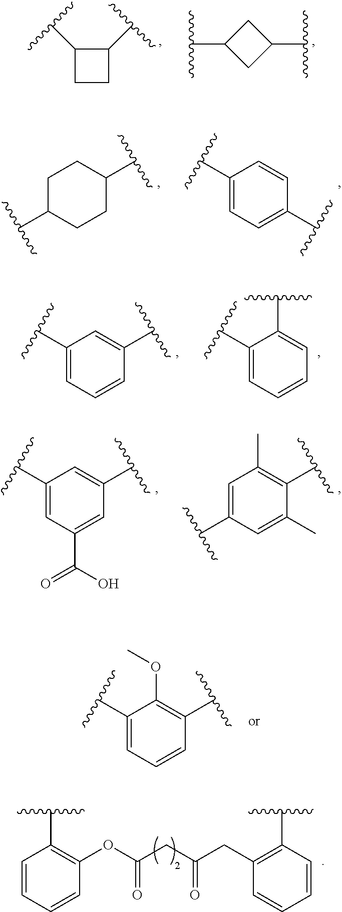

In some embodiments, R^{D} or R^{E} is (CH₂)₆. In some embodiments, R^{D} or R^{E} is (CH₂)₇. In some embodiments, R^{D} or R^{E} is

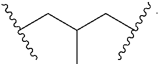 [SMILES: *CC(C)C*]

In some embodiments, R^{D} or R^{E} is

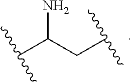 [SMILES: *CC(*)N]

In some embodiments, R^{D} or R^{E} is

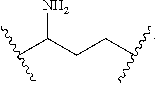 [SMILES: *CCC(*)N]

In some embodiments, R^{D} or R^{E} is

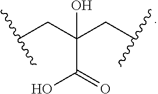 [SMILES: *CC(O)(C*)C(=O)O]

In some embodiments, R^{D} or R^{E} is

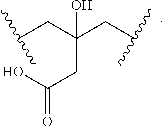 [SMILES: *CC(O)(C*)CC(=O)O]

In some embodiments, R^{D} or R^{E} is

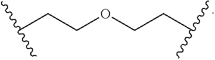 [SMILES: *CCOCC*]

In some embodiments, R^{D} or R^{E} is

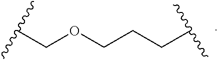 [SMILES: *CCCOC*]

In some embodiments, R^{D} or R^{E} is

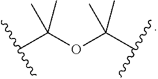 [SMILES: *C(C)(C)OC(*)(C)C]

In some embodiments, R^{D} or R^{E} is

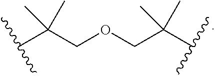 [SMILES: *C(C)(C)COCC(*)(C)C]

In some embodiments, R^{D} or R^{E} is —(CH₂)₂—(OCH₂CH₂)₂—. In some embodiments, R^{D} or R^{E} is —(CH₂)₂—(OCH₂CH₂)₃—. In some embodiments, R^{D} or R^{E} is

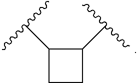 [SMILES: *C1CCC1*]

In some embodiments, R^{D} or R^{E} is

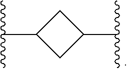 [SMILES: *C1CC(*)C1]

In some embodiments, R^{D} or R^{E} is

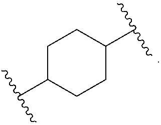 [SMILES: *C1CCC(*)CC1]

In some embodiments, R^{D} or R^{E} is

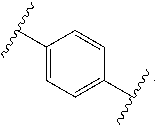 [SMILES: *c1ccc(*)cc1]

In some embodiments, R^{D} or R^{E} is

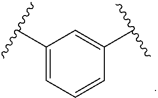 [SMILES: *c1cccc(*)c1]

In some embodiments, R^{D} or R^{E} is

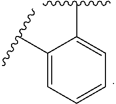 [SMILES: *c1ccccc1*]

In some embodiments, R^{D} or R^{E} is

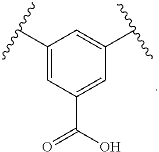 [SMILES: *c1cc(*)cc(C(=O)O)c1]

In some embodiments, R^{D} or R^{E} is

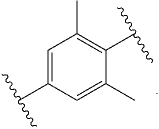 [SMILES: *c1cc(C)c(*)c(C)c1]

In some embodiments, R^{D} or R^{E} is

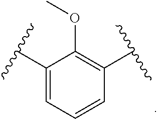 [SMILES: *c1cccc(*)c1OC]

In some embodiments, R^{D} or R^{E} is

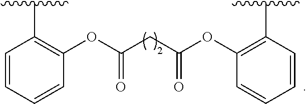

In some embodiments, when the compound of formula (Ia) is deuterated, the compound has a structure of formula (D-Ia),

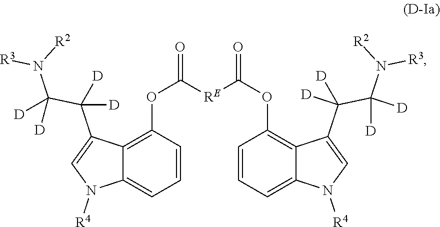

or a pharmaceutically acceptable salt thereof, wherein R^{E}, R², R³ and R⁴ are defined in formula (Ia).

In some embodiments, the compound of formula (I) is a compound of formula (Ib)

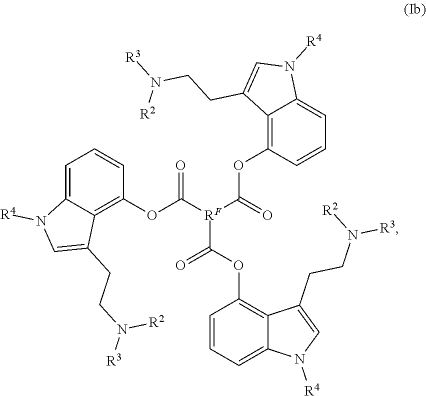

or a pharmaceutically acceptable salt or deuterated form thereof, wherein
each R² and R³ are independently alkyl;
each R⁴ is independently H or C(═O)Oalkyl;
R^{F} is a trivalent group selected from alkylene substituted with OH or C(═O)alkyl, alkenylene, cycloalkylene or arylene.

In some embodiments, R^{F} is a trivalent alkylene. In some embodiments, R^{F} is a trivalent C₃-C₈ alkylene. In some embodiments, R^{F} is a trivalent C₃-C₆ alkylene. In some embodiments, the trivalent alkylene is substituted with OH or C(═O)alkyl. In some embodiments, the trivalent alkylene is substituted with OH or C(═O)CH₃. In some embodiments, R^{F} is

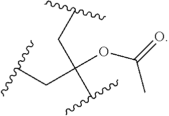 [SMILES: *CC(*)(C*)OC(C)=O]

In some embodiments, R^{F} is

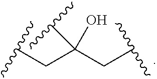 [SMILES: *CC(*)(O)C*]

In some embodiments, R^{F} is a trivalent alkenylene. In some embodiments, R^{F} is a trivalent C₃-C₈ alkenylene. In some embodiments, R^{F} is a trivalent C₃-C₆ alkenylene. In some embodiments, R^{F} is a trivalent C₃-C₅ alkenylene. In some embodiments, R^{F} is

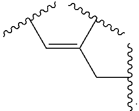 [SMILES: */C=C(\*)C*]

In some embodiments, R^{F} is a trivalent cycloalkylene. In some embodiments, R^{F} is a trivalent C₃-C₈ cycloalkylene. In some embodiments, R^{F} is a trivalent C₃-C₆ cycloalkylene. In some embodiments, R^{F} is a trivalent C₆ cycloalkylene. In some embodiments, R^{F} is

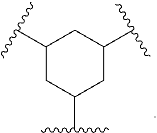 [SMILES: *C1CC(*)CC(*)C1]

In some embodiments, R^{F} is a trivalent arylene. In embodiments, R^{F} is trivalent phenylene. In embodiments, R^{F} is

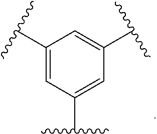 [SMILES: *c1cc(*)cc(*)c1]

In some embodiments, when the compound of formula (Ib) is deuterated, the compound has a structure of formula (D-Ib)

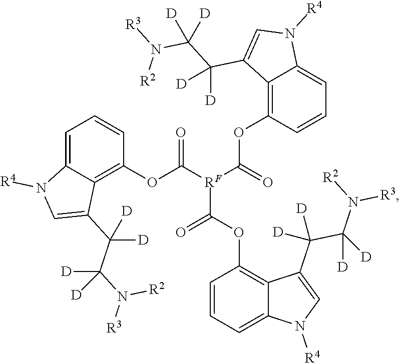

or a pharmaceutically acceptable salt thereof, wherein R^{F}, R², R³ and R⁴ are as defined in formula (Ib).

In some embodiments, this disclosure provides a compound of formula (II),

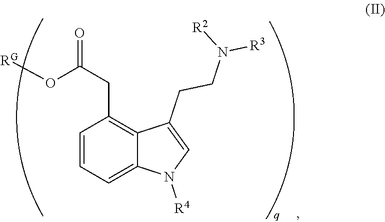

or a pharmaceutically acceptable salt or deuterated form thereof,
wherein:
R² and R³ are independently alkyl;
R⁴ is H or C(═O)Oalkyl;
q is 2, 3 or 4;
R^{G} is —(CH₂)ₙ—(OCH₂CH₂)ₘ—, or a divalent, trivalent or tetravalent alkylene or alkenylene;
n is 1, 2, 3, 4, 5, 6, 7 or 8; and
m is 0, 1, 2 3, 4, 5, 6, 7 or 8.

In some embodiments of formula (II), when q is 2 and R² and R³ are each CH₃, R^{G} is not —(CH₂)₂— or —(CH₂)₃—.

In some embodiments, R^{G} is —(CH₂)ₙ—(OCH₂CH₂)ₘ—, or a trivalent or tetravalent alkylene, or divalent, trivalent or tetravalent alkenylene.

In some embodiments, R^{G} is —(CH₂)ₙ—(OCH₂CH₂)ₘ—. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, m is 1. In some embodiments, m is 2. In some embodiments, m is 3. In some embodiments, n is 1-3 and m is 1-4. In some embodiments, n is 2 and m is 2 or 3. In some embodiment, n is 2 and m is 2.

In some embodiments, R^{G} is —(CH₂)₂—(OCH₂CH₂)₂—,

In some embodiments, R^{G} is a divalent alkylene. In some embodiments, R^{G} is a divalent C₁-C₁₂ alkylene. some embodiments, R^{G} is a divalent C₁-C₁₀ alkylene. In some embodiments, R^{G} is a divalent C₁-C₈ alkylene. In embodiments, R^{G} is —CH₂—. In embodiments, R^{G} is —(CH₂)₄—. In embodiments, R^{G} is —(CH₂)₅—. In embodiments, R^{G} is —(CH₂)₆—. In embodiments, R^{G} is —(CH₂)₇—. In embodiments, R^{G} is —(CH₂)₈—.

In some embodiments, R^{G} is a trivalent alkylene. In embodiments, R^{G} is a trivalent C₁-C₈ alkylene. In embodiments, R^{G} is a trivalent C₁-C₈ alkylene. In embodiments, R^{G} is a trivalent C₁-C₆ alkylene. In embodiments, R^{G} is a trivalent C₂-C₆ alkylene. In embodiments, R^{G} is a trivalent C₁-C₅ alkylene. In embodiments, R^{G} is a trivalent C₂-C₅ alkylene. In embodiments, R^{G} is a trivalent C₂-C₄ alkylene. In embodiments, R^{G} is

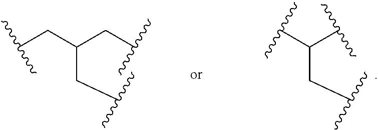 [SMILES: *CC(C*)C* ; *CC(*)*]

In embodiments, R^{G} is

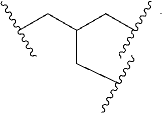 [SMILES: *CC(C*)C*]

In embodiments, R^{G} is

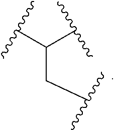 [SMILES: *CC(*)*]

In some embodiments, R^{G} is a tetravalent alkylene. In embodiments, R^{G} is a tetravalent C₁-C₈ alkylene. In embodiments, R^{G} is a tetravalent C₃-C₈ alkylene. In embodiments, R^{G} is a tetravalent C₃-C₆ alkylene. In embodiments, R^{G} is a tetravalent C₅ alkylene. In embodiments, R^{G} is

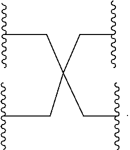 [SMILES: *CC(C*)(C*)C*]

In some embodiments, R^{G} is —CH₂—, —(CH₂)₄—, —(CH₂)₅—, —(CH₂)₆—, —(CH₂)₇—, —(CH₂)₈— —(CH₂)₂—(OCH₂CH₂)₂—,

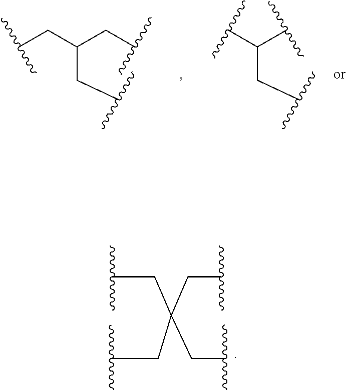 [SMILES: *CC(C*)C* ; *CC(*)* ; *CC(C*)(C*)C*]

In some embodiments, when the compound of formula (II) is deuterated, the compound has a structure of formula (D-II),

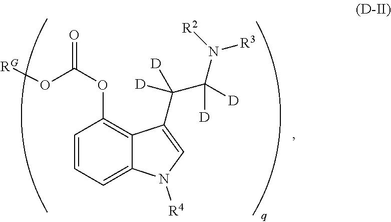

or a pharmaceutically acceptable salt thereof, wherein R^{G}, R², R³ and R⁴ are as defined in formula (II).

In some embodiments, the compound of formula (II) is a compound of formula (IIa),

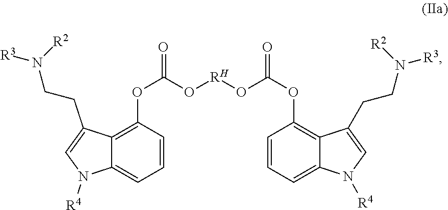

or a pharmaceutically acceptable salt or deuterated form thereof,
wherein:
   each R² and R³ are independently alkyl;
   each R⁴ is independently H or C(═O)Oalkyl;
   R^{H} is —(CH₂)ₙ—(OCH₂CH₂)ₘ—;
   n is 1, 2, 3, 4, 5, 6, 7 or 8, and
   m is 0, 1, 2 or 3.

In embodiments, compound of formula (IIa) is a compound of formula (IIa-D) is

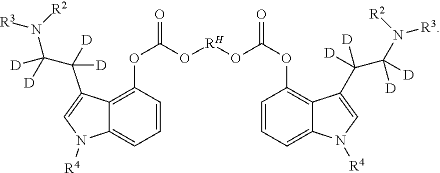

In some embodiments, R^{H} is —(CH₂)ₙ—(OCH₂CH₂)ₘ—. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, m is 1. In some embodiments, m is 2. In some embodiments, m is 3. In some embodiments, n is 1-3 and m is 1-4. In some embodiments, n is 2 and m is 2 or 3. In some embodiment, n is 2 and m is 2.

In some embodiments, R^{H} is —(CH₂)₂—(OCH₂CH₂)₂—,

In some embodiments, R^{G} or R^{H} is —(CH₂)₂—, —(CH₂)₄—, —(CH₂)₅—, —(CH₂)₆—, —(CH₂)₇—, —(CH₂)₈— or —(CH₂)₂—(OCH₂CH₂)₂—.

In embodiments, when the compound of formula (IIa) is a deuterated compound, the compound has a structure of formula (D-IIa),

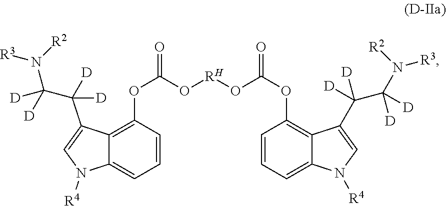

or a pharmaceutically acceptable salt thereof, wherein R^{H}, R², R³ and R⁴ are as defined in formula (IIa).

In some embodiments, the compound of formula (II) is a compound of formula (IIb),

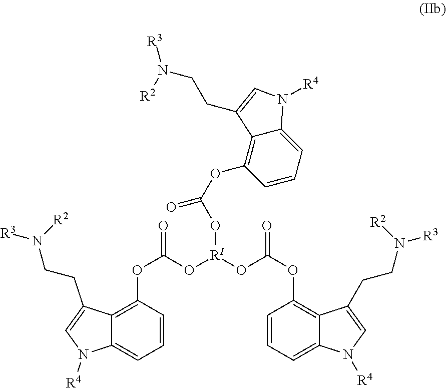

or a pharmaceutically acceptable salt or deuterated form thereof,
wherein:
   each R² and R³ are independently alkyl;
   each R⁴ is independently H or C(═O)Oalkyl;
   R^{I} is a trivalent alkylene group.

In some embodiments, R^{G} or R^{I} is a trivalent C₁₋₆ alkylene group. In some embodiments, R^{G} or R^{I} is a trivalent C₂₋₆ alkylene group. In some embodiments, R^{G} or R^{I} is a trivalent C₂₋₅ alkylene group. In some embodiments, R^{G} or R^{I} is a trivalent C₂₋₄ alkylene group. In some embodiments, R^{G} or R^{I} is a trivalent C₂ alkylene group. In some embodiments, R^{G} or R^{I} is a trivalent C₄ alkylene group. In some embodiments, R^{G} or R^{I} is

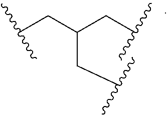 [SMILES: *CC(C*)C*]

In some embodiments, R^{G} or R^{I} is

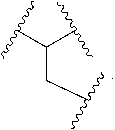 [SMILES: *CC(*)*]

In some embodiments, R^{G} or R^{I} is

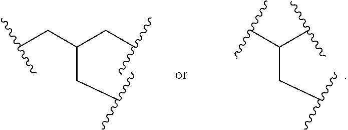 [SMILES: *CC(C*)C* ; *CC(*)*]

In some embodiments, when the compound of formula (IIb) is deuterated, the compound has the structure of formula (D-IIb)

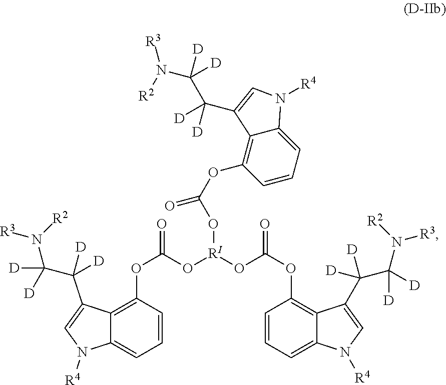

or a pharmaceutically acceptable salt thereof, wherein R¹, R², R³ and R⁴ are defined in formula (IIb).

In some embodiments, the compound is a compound of formula (IIc),

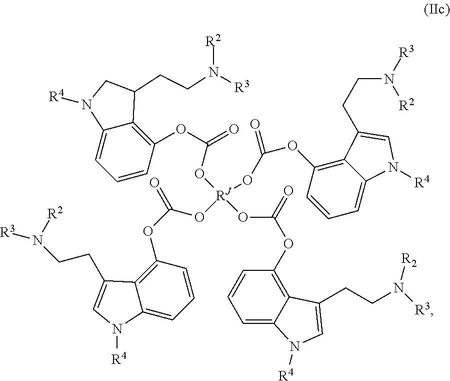

or a pharmaceutically acceptable salt or deuterated form thereof,
wherein:
   R² and R³ are independently alkyl;
   R⁴ is H or C(═O)Oalkyl;
   R^{J} is a tetravalent alkylene group.

In some embodiments, R^{G} or R^{J} is a tetravalent C₁₋₆ alkylene group. In some embodiments, R^{G} or R^{J} is a tetravalent C₂₋₆ alkylene group. In some embodiments, R^{G} or R^{J} is a tetravalent C₂₋₅ alkylene group. In some embodiments, R^{G} or R^{J} is a tetravalent C₅ alkylene group.

In some embodiments, R^{G} or R^{J} is

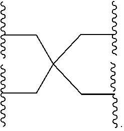

In some embodiments, when the compound of formula (IIc) is deuterated, the compound has the structure of formula (D-IIc),

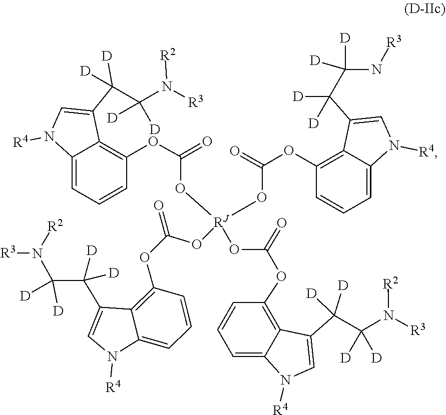

or a pharmaceutically acceptable salt thereof, wherein G^{J}, R², R³ and R⁴ are defined in formula (IIc).

In some embodiments, this disclosure provides a compound of formula (III),

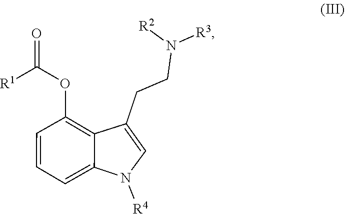

or a pharmaceutically acceptable salt thereof or deuterated form thereof, wherein:
R² and R³ are independently alkyl;
R⁴ is H or C(═O)Oalkyl;
R¹ is alkylene-C(═O)OH wherein the alkylene is optionally substituted with OH, C(═O)OH, —OC(═O)alkyl or NH₂, alkenylene-C(═O)OH wherein the alkenylene is optionally substituted with C(═O)OH, cycloalkylene-C(═O)OH wherein the cycloalkylene is optionally substituted with C(═O)OH, arylene-C(═O)OH wherein the arylene is optionally substituted with alkyl, Oalkyl or C(═O)OH, —(CH₂CH₂O)ₘ—(CH₂CH₂)ₙ—C(═O)OH, alkylene-O-alkylene-C(═O)OH, arylene-O(C═O)-alkylene-C(═O)O-arylene-C(═O)OH, O-alkylene-OH wherein the alkylene is optionally substituted with OH, or —(OCH₂CH₂)ₘ—OH;
m is 1, 2, 3; and
n is 1, 2, 3.

In some embodiments of formula (III), when R² and R³ are each alkyl, then R¹ is not (CH₂)₂C(═O)OH, (CH₂)₃C(═O)OH, —CH═CHC(═O)OH or

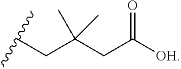 [SMILES: *CC(C)(C)CC(=O)O]

In some embodiments, R¹ is alkenylene-C(═O)OH wherein the alkenylene is optionally substituted with C(═O)OH, cycloalkylene-C(═O)OH wherein the cycloalkylene is optionally substituted with C(═O)OH, arylene-C(═O)OH wherein the arylene is optionally substituted with alkyl, Oalkyl or C(═O)OH, —(CH₂CH₂O)ₘ—(CH₂CH₂)ₙ—C(═O)OH, alkylene-O-alkylene-C(═O)OH, arylene-O(C═O)-alkylene-C(═O)O-arylene-C(═O)OH, O-alkylene-OH wherein the alkylene is optionally substituted with OH, or —(OCH₂CH₂)ₘ—OH. In some embodiments, R¹ is C₁₋₁₀alkylene-C(═O)OH wherein the alkylene is optionally substituted with OH, C(═O)OH, —OC(═O)alkyl or NH₂, C₁₋₆alkenylene-C(═O)OH wherein the alkenylene is optionally substituted with C(═O)OH, C₃₋₆cycloalkylene-C(═O)OH wherein the cycloalkylene is optionally substituted with C(═O)OH, arylene-C(═O)OH wherein the arylene is optionally substituted with alkyl, Oalkyl or C(═O)OH, —(CH₂CH₂O)ₘ—(CH₂CH₂)ₙ—C(═O)OH, C₁₋₆alkylene-O—C₁₋₆alkylene-C(═O)OH, arylene-O(C═O)-alkylene-C(═O)O-arylene-C(═O)OH, O—C₁₋₁₀alkylene-OH wherein the alkylene is optionally substituted with OH, or —(OCH₂CH₂)ₘ—OH.

In some embodiments, R¹ is C₁₋₁₀alkylene-C(═O)OH wherein the alkylene is optionally substituted with OH, C(═O)OH, —OC(═O)alkyl or NH₂.

In some embodiments, R¹ is C₁₋₆alkenylene-C(═O)OH wherein the alkenylene is optionally substituted with C(═O)OH.

In some embodiments, R¹ is C₃₋₆cycloalkylene-C(═O)OH wherein the cycloalkylene is optionally substituted with C(═O)OH,

In some embodiments, R¹ is arylene-C(═O)OH wherein the arylene is optionally substituted with alkyl, Oalkyl or C(═O)OH.

In some embodiments, R¹ is —(CH₂CH₂O)ₘ—(CH₂CH₂)ₙ—C(═O)OH. In some embodiments, m is 1, 2, 3 and n is 1.

In some embodiments, R¹ is C₁₋₆alkylene-O—C₁₋₆alkylene-C(═O)OH. In some embodiments, R¹ is C₁₋₅alkylene-O—C₁₋₅alkylene-C(═O)OH. In some embodiments, R¹ is C₁₋₄alkylene-O—C₁₋₄alkylene-C(═O)OH.

In some embodiments, R¹ is arylene-O(C═O)-alkylene-C(═O)O-arylene-C(═O)OH.

In some embodiments, R¹ is O—C₁₋₁₀alkylene-OH wherein the alkylene is optionally substituted with OH or —(OCH₂CH₂)ₘ—OH

In embodiments, R¹ is —(OCH₂CH₂)ₘ—OH.

In some embodiments, R¹ is

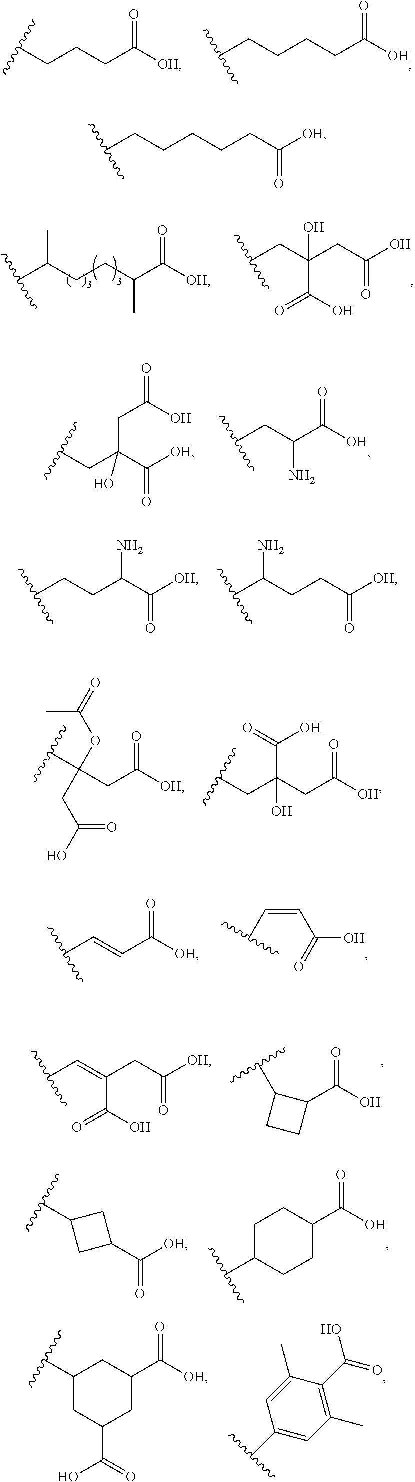

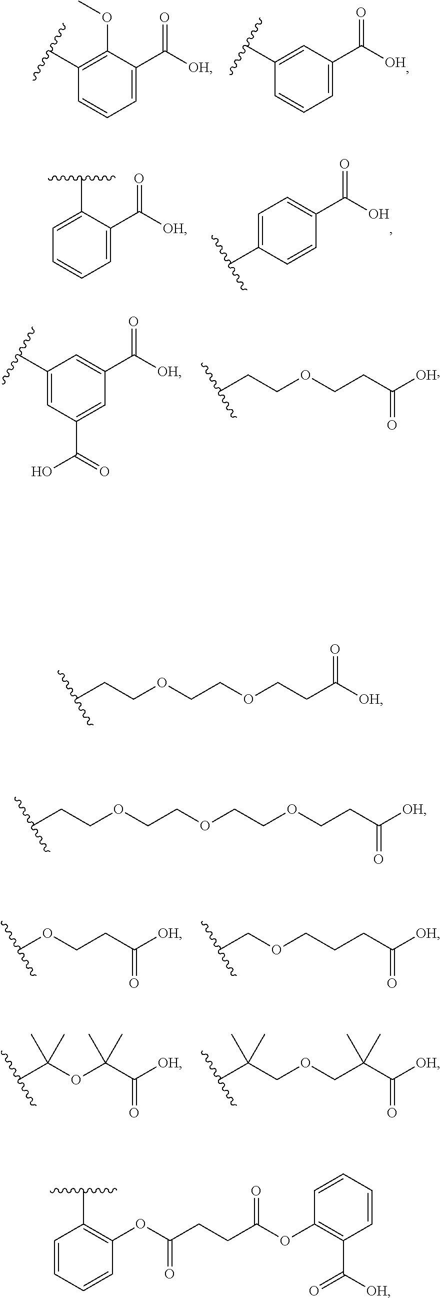

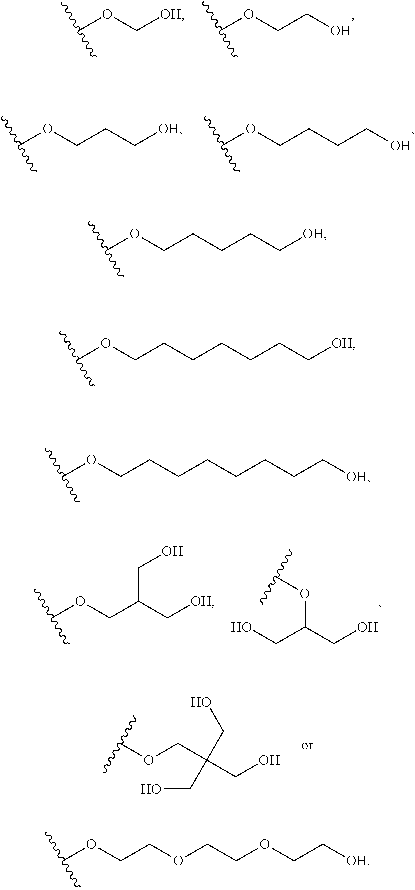

In some embodiments, R¹ is

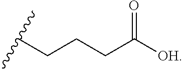 [SMILES: *CCCC(=O)O]

In some embodiments, R¹ is

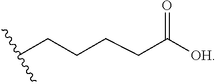 [SMILES: *CCCCC(=O)O]

In some embodiments, R¹ is

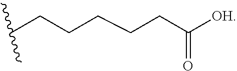 [SMILES: *CCCCCC(=O)O]

In some embodiments. In some embodiments, R¹ is R¹ is

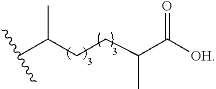

In some embodiments, R¹ is

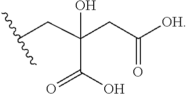 [SMILES: *CC(O)(CC(=O)O)C(=O)O]

In some embodiments, R¹ is

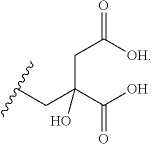 [SMILES: *CC(O)(CC(=O)O)C(=O)O]

In some embodiments, R¹ is

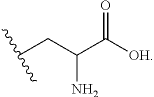 [SMILES: *CC(N)C(=O)O]

In some embodiments, R¹ is

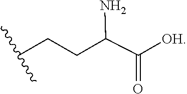 [SMILES: *CCC(N)C(=O)O]

In some embodiments, R¹ is

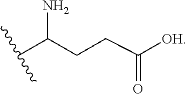 [SMILES: *C(N)CCC(=O)O]

In some embodiments, R¹ is

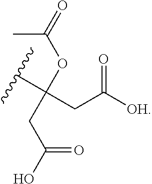 [SMILES: *C(CC(=O)O)(CC(=O)O)OC(C)=O]

In some embodiments, R¹ is

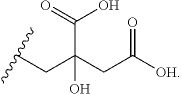 [SMILES: *CC(O)(CC(=O)O)C(=O)O]

In some embodiments, R¹ is

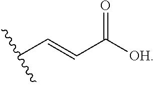 [SMILES: */C=C/C(=O)O]

In some embodiments, R¹ is

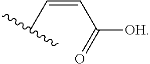 [SMILES: */C=C\C(=O)O]

In some embodiments, R¹ is

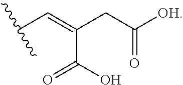 [SMILES: */C=C(/CC(=O)O)C(=O)O]

In some embodiments, R¹ is

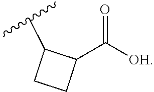 [SMILES: *C1CCC1C(=O)O]

In some embodiments, R¹ is

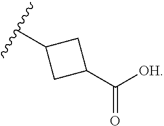 [SMILES: *C1CC(C(=O)O)C1]

In some embodiments, R¹ is

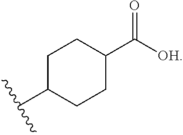 [SMILES: *C1CCC(C(=O)O)CC1]

In some embodiments, R¹ is

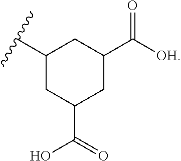 [SMILES: *C1CC(C(=O)O)CC(C(=O)O)C1]

In some embodiments, R¹ is

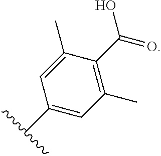 [SMILES: *c1cc(C)c(C(=O)O)c(C)c1]

In some embodiments, R¹ is

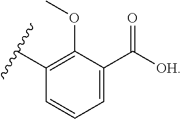 [SMILES: *c1cccc(C(=O)O)c1OC]

In some embodiments, R¹ is

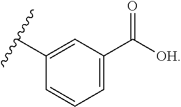 [SMILES: *c1cccc(C(=O)O)c1]

In some embodiments, R¹ is

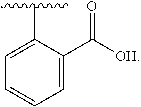 [SMILES: *c1ccccc1C(=O)O]

In some embodiments, R¹ is

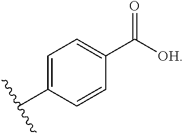 [SMILES: *c1ccc(C(=O)O)cc1]

In some embodiments, R¹ is

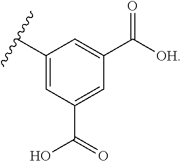 [SMILES: *c1cc(C(=O)O)cc(C(=O)O)c1]

In some embodiments, R¹ is

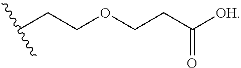 [SMILES: *CCOCCC(=O)O]

In some embodiments, R¹ is

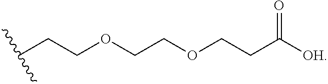 [SMILES: *CCOCCOCCC(=O)O]

In some embodiments, R¹ is

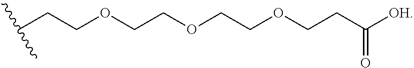 [SMILES: *CCOCCOCCOCCC(=O)O]

In some embodiments, R¹ is

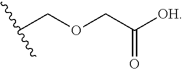 [SMILES: *COCC(=O)O]

In some embodiments, R¹ is

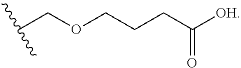 [SMILES: *COCCCC(=O)O]

In some embodiments, R¹ is

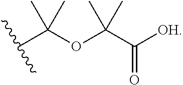 [SMILES: *C(C)(C)OC(C)(C)C(=O)O]

In some embodiments, R¹ is

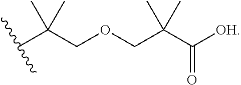 [SMILES: *C(C)(C)COCC(C)(C)C(=O)O]

In some embodiments, R¹ is

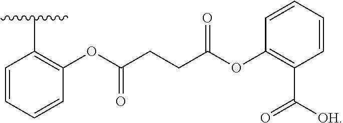 [SMILES: *c1ccccc1OC(=O)CCC(=O)Oc1ccccc1C(=O)O]

In some embodiments, R¹ is

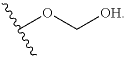 [SMILES: *OCO]

In some embodiments, R¹ is

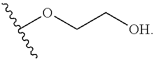 [SMILES: *OCCO]

In some embodiments, R¹ is

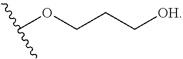 [SMILES: *OCCCO]

In some embodiments, R¹ is

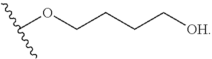 [SMILES: *OCCCCO]

In some embodiments, R¹ is

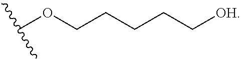 [SMILES: *OCCCCCO]

In some embodiments, R¹ is

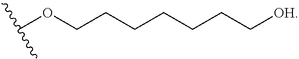 [SMILES: *OCCCCCCCO]

In some embodiments, R¹ is

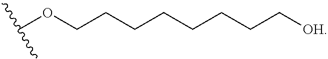 [SMILES: *OCCCCCCCCO]

In some embodiments, R¹ is

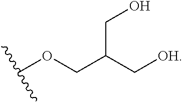 [SMILES: *OCC(CO)CO]

In some embodiments, R¹ is

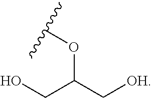 [SMILES: *OC(CO)CO]

In some embodiments, R¹ is

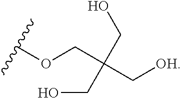 [SMILES: *OCC(CO)(CO)CO]

In some embodiments, R¹ is

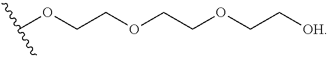 [SMILES: *OCCOCCOCCO]

In some embodiments, when the compound of formula (III) is deuterated, the compound has the structure of formula (D-III),

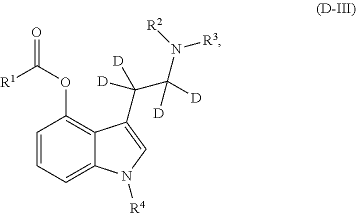

or a pharmaceutically acceptable salt thereof, wherein R¹, R², R³ and R⁴ are defined in formula (III).

In some embodiments of formula (I), (Ia), (Ib), (II), (Iia), (Iib), (IIc), (III), (D-I), (D-Ia), (D-Ib), (D-II), (D-Iia), (D-Iib), (D-IIc) or (D-III), R² and R³ are independently C₁₋₆ alkyl.

In some embodiments of formula (I), (Ia), (Ib), (II), (Iia), (Iib), (IIc), (III), (D-I), (D-Ia), (D-Ib), (D-II), (D-Iia), (D-lib), (D-IIc) or (D-III), R² and R³ are independently deuterated C₁₋₆ alkyl.

In some embodiments of formula (I), (Ia), (Ib), (II), (Iia), (Iib), (IIc), (III), (D-I), (D-Ia), (D-Ib), (D-II), (D-Iia), (D-lib), (D-IIc) or (D-III), R² and R³ are independently perdeuterated C₁₋₆ alkyl.

In some embodiments of formula (I), (Ia), (Ib), (II), (Iia), (Iib), (IIc), (III), (D-I), (D-Ia), (D-Ib), (D-II), (D-Iia), (D-Iib), (D-IIc) or (D-III), R² and R³ are —CH₃.

In some embodiments of formula (I), (Ia), (Ib), (II), (Iia), (Iib), (IIc), (III), (D-I), (D-Ia), (D-Ib), (D-II), (D-Iia), (D-Iib), (D-IIc) or (D-III), R² and R³ are deuterated-CH₃.

In some embodiments of formula (I), (Ia), (Ib), (II), (Iia), (Iib), (IIc), (III), (D-I), (D-Ia), (D-Ib), (D-II), (D-Iia), (D-Iib), (D-IIc) or (D-III), R² and R³ are deuterated-CD₃.

In some embodiments of formula (I), (Ia), (Ib), (II), (Iia), (Iib), (IIc), (III), (D-I), (D-Ia), (D-Ib), (D-II), (D-Iia), (D-Iib), (D-IIc) or (D-III), R⁴ is H or C(═O)OC₁₋₆ alkyl.

In some embodiments of formula (I), (Ia), (Ib), (II), (Iia), (Iib), (IIc), (III), (D-I), (D-Ia), (D-Ib), (D-II), (D-Iia), (D-lib), (D-IIc) or (D-III), R⁴ is H or C(═O)OCH₃.

In some embodiments of formula (I), (Ia), (Ib), (II), (Iia), (Iib), (IIc), (III), (D-I), (D-Ia), (D-Ib), (D-II), (D-Iia), (D-Iib), (D-IIc) or (D-III), R⁴ is H.

In some embodiments of formula (I), (Ia), (Ib), (II), (Iia), (Iib), (IIc), (III), (D-I), (D-Ia), (D-Ib), (D-II), (D-Iia), (D-Iib), (D-IIc) or (D-III), R² and R³ are independently CH₃, and R⁴ is H.

In some embodiments, this disclosure provides a compound selected from Table 1, Table 2, Table 3, or a pharmaceutically acceptable salt thereof, or a deuterated form thereof.

In some embodiments, this disclosure provides a compound selected from Table 1, Table 2, or a pharmaceutically acceptable salt thereof, or a deuterated form thereof.

In some embodiments, this disclosure provides a compound selected from Table 1, a pharmaceutically acceptable salt thereof, or a deuterated form thereof.

In some embodiments, this disclosure provides a compound selected from Table 4, a pharmaceutically acceptable salt thereof, or a deuterated form thereof.

In some embodiments, this disclosure provides a method of treating a disease comprising subcutaneously administrating a pharmaceutical composition comprising a compound selected from Table 1, Table 2, Table 3, Table 4, or a pharmaceutically acceptable salt thereof, or a deuterated form thereof.

In some embodiments, this disclosure provides a method of treating a disease comprising subcutaneously administrating a pharmaceutical composition comprising a compound of formula (I), formula (II), or formula (III) or a pharmaceutically acceptable salt thereof, or a deuterated form thereof,

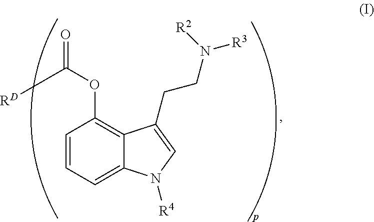

wherein
   R² and R³ are independently alkyl;
   R⁴ is H or C(═O)Oalkyl;
   p is 2 or 3;
   R^{D} is a divalent or trivalent group selected from alkyl, alkenyl, alkylene-O-alkylene, —(CH₂)ₙ—(OCH₂CH₂)ₘ—, cycloalkyl, heterocyclyl, aryl, heteroaryl or

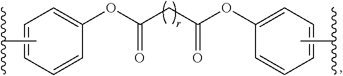

each of which is optionally substituted with OH, Oalkyl, NH₂, NH(alkyl), N(alkyl)₂, cycloalkyl, COOH, C(═O)Oalkyl, or C(═O)alkyl;
n is 1, 2, 3, 4, 5, 6, 7 or 8; m is 1, 2 or 3; and
r is 1, 2, 3;
   or

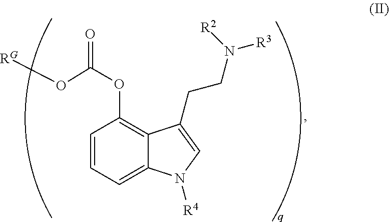

or a pharmaceutically acceptable salt or deuterated form thereof,
   wherein:
R² and R³ are independently alkyl;
R⁴ is H or C(═O)Oalkyl;
q is 2, 3 or 4;
R^{G} is —(CH₂)ₙ—(OCH₂CH₂)ₘ—, or a trivalent or tetravalent alkyl group; wherein n is 1, 2, 3, 4, 5, 6, 7 or 8, and m is 0, 1, 2 or 3,
or

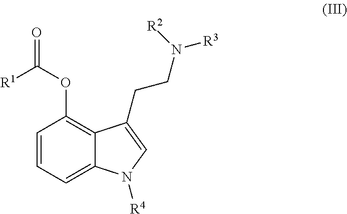

or a pharmaceutically acceptable salt thereof or deuterated form thereof, wherein:
   R² and R³ are independently alkyl;
   R⁴ is H or C(═O)Oalkyl;
   R¹ is alkylene-C(═O)OH wherein the alkylene is optionally substituted with OH, C(═O)OH, —OC(═O)alkyl or NH₂, alkenylene-C(═O)OH wherein the alkenylene is optionally substituted with C(═O)OH, cycloalkylene-C(═O)OH wherein the cycloalkylene is optionally substituted with C(═O)OH, arylene-C(═O)OH wherein the arylene is optionally substituted with alkyl, Oalkyl or C(═O)OH, —(CH₂CH₂O)ₘ—(CH₂CH₂)ₙ—C(═O)OH, alkylene-O-alkylene-C(═O)OH, arylene-O(C═O)-alkylene-C(═O)O-arylene-C(═O)OH, O-alkylene-OH wherein the alkylene is optionally substituted with OH, or —(OCH₂CH₂)ₘ—OH;
   m is 1, 2, 3,
   n is 1, 2, 3.

In some embodiments, the present disclosure provides a compound of formula (I):

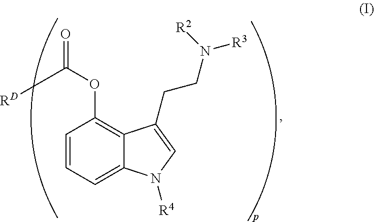

or a pharmaceutically acceptable salt thereof or deuterated form thereof, wherein:
   R² and R³ are independently alkyl;
   R⁴ is H or C(═O)Oalkyl;
   p is 2 or 3;
   R^{D} is a divalent or trivalent group selected from alkylene, alkenylene, alkylene-O-alkylene, —(CH₂)ₙ—(OCH₂CH₂)ₘ—, cycloalkylene, or arylene, or

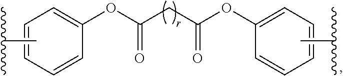

each of which is optionally substituted with 1-4 groups selected from halogen, OH, Oalkyl, alkyl, NH₂, NH(alkyl), N(alkyl)₂, C(═O)OH, C(═O)Oalkyl, OC(═O)alkyl, cycloalkyl, or C(═O)alkyl;
n is 1, 2, 3, 4, 5, 6, 7 or 8;
m is 1, 2, 3, 4, 5, 6, 7 or 8;
r is 1, 2 or 3;
provided that when R² and R³ are each alkyl, then R^{D} is not CH₂, (CH₂)₂, (CH₂)₃, (CH₂)₄, (CH₂)₅, (CH₂)₈,

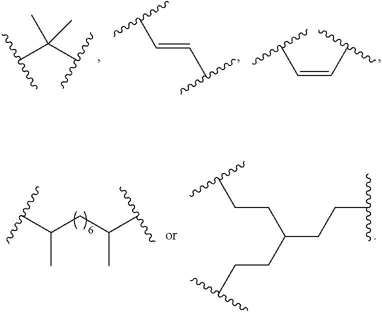

In some embodiments of formula (I), when R² and R³ are each alkyl, then R^{D} is not CH₂, (CH₂)₂, (CH₂)₃, (CH₂)₄, (CH₂)₅, (CH₂)₈,

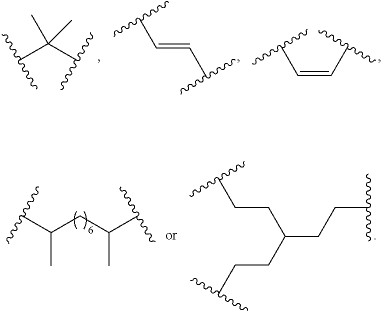

In some embodiments, R^{D} is a divalent or trivalent group selected from C₁₋₁₂ alkylene, C₁₋₆alkylene-O—C₁₋₆alkylene, —(CH₂)ₙ—(OCH₂CH₂)ₘ—, C₃₋₈cycloalkylene, arylene or

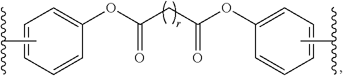

each of which is optionally substituted with 1-4 groups selected from with C₁₋₃ alkyl, OH, OCH₃, NH₂, cycloalkyl, COOH, or C(═O)alkyl.

In some embodiments, the compound is a compound of formula (Ia),

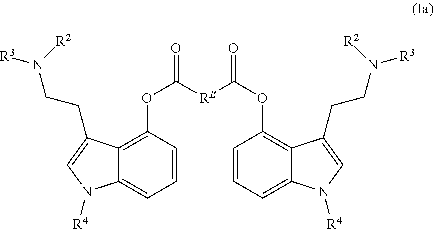

or a pharmaceutically acceptable salt or deuterated thereof,
wherein:
   each R² and R³ are independently alkyl;
   each R⁴ is independently H or C(═O)Oalkyl;
   R^{E} is a divalent group selected from alkylene, cycloalkylene, arylene, alkylene-O-alkylene, —(CH₂)ₙ—(OCH₂CH₂)^{m}— or

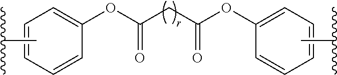

each of which is optionally substituted with one or more groups selected from halogen, alkyl, Oalkyl, NH₂, NHC₁₋₆alkyl, NC(₁₋₆alkyl)₂, COOH, and C(═O)Oalkyl;
n is 1, 2, or 3;
m is 1, 2 or 3; and
r is 1, 2 or 3.

In embodiments, R^{E} is a divalent group selected from C₁₋₁₂ alkylene, C₃₋₈cycloalkylene, arylene, C₁₋₆alkylene-O—C₁₋₆alkylene, —(CH₂)ₙ—(OCH₂CH₂)ₘ— or

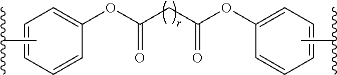

each of which is optionally substituted with one or more groups selected from halogen, C₁₋₃alkyl, Oalkyl, NH₂, NHC₁₋₆alkyl, NC(₁₋₆alkyl)₂, COOH, and C(═O)Oalkyl.

In some embodiments, R^{D} or R^{E} is

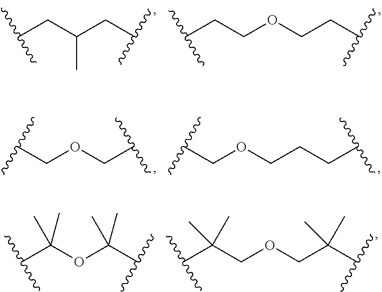

—(CH₂)₂—(OCH₂CH₂)₂—, —(CH₂)₂—(OCH₂CH₂)₃—,

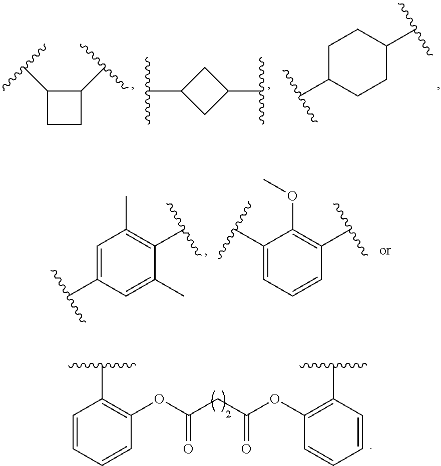

In some embodiments, the compound of formula (I) is a compound of formula (Ib)

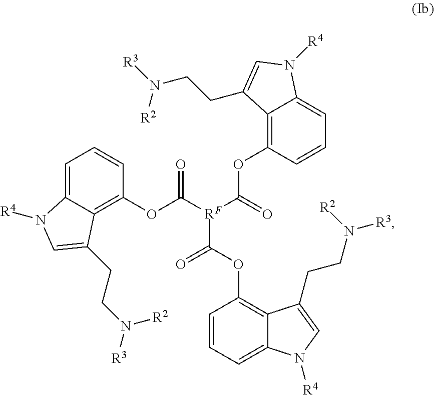

or a pharmaceutically acceptable salt or deuterated form thereof, wherein
each R² and R³ are independently alkyl;
each R⁴ is independently H or C(═O)Oalkyl;
R^{F} is a trivalent cycloalkylene or alkylene group.

In some embodiments, R^{F} is a trivalent C₃₋₈cycloalkylene.

In some embodiments, R^{F} is

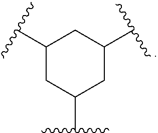 [SMILES: *C1CC(*)CC(*)C1]

In certain embodiments, R^{F} is

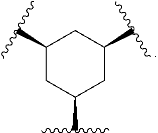 [SMILES: *[C@H]1C[C@@H](*)C[C@@H](*)C1]

In some embodiments, the present disclosure provides A compound of formula (II),

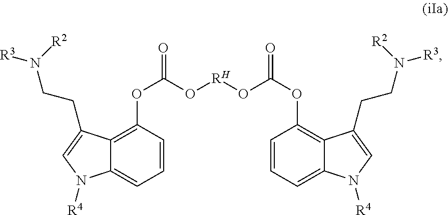

or a pharmaceutically acceptable salt or deuterated form thereof,
wherein:
   each R² and R³ are independently alkyl;
   each R⁴ is independently H or C(═O)Oalkyl;
   R^{H} is —(CH₂)ₙ—(OCH₂CH₂)ₘ— or alkylene;
   n is 1, 2, 3, 4, 5, 6, 7, 8, 9, 10, 11 or 12, and
   m is 1, 2 or 3;
   provided that when q is 2 and R² and R³ are each CH₃, R^{H} is not —(CH₂)₃—.

In some embodiments of compound of formula (II), n is 1, 2, 3, 4, 5, 6, 7, 8, and m is 1, 2 or 3.

In some embodiments, R^{H} is —(CH₂)₅—, —(CH₂)₈— or —(CH₂)₂—(OCH₂CH₂)₂—.

In some embodiments, R² and R³ are independently C₁₋₆ alkyl.

In some embodiments, R² and R³ are —CH₃.

In some embodiments, R⁴ is H or C(═O)OC₁₋₆ alkyl.

In some embodiments, R⁴ is H or C(═O)OCH₃.

In some embodiments, R⁴ is H.

In some embodiments, wherein R² and R³ are independently CH₃, and R⁴ is H.

In embodiments, the disclosure provides compounds of Table 1.

In embodiments, the disclosure provides compounds of Table 1 and Table 2.

In embodiments, the disclosure provides compounds of Table 4.

In some embodiments, the compound disclosed herein has a human plasma psilocin release efficiency of greater than 50%. In some embodiments, the compound disclosed herein has a human plasma psilocin release efficiency of greater than 60%. In some embodiments, the compound disclosed herein has a human plasma psilocin release efficiency of greater than 70%. In some embodiments, the compound disclosed herein has a human plasma psilocin release efficiency of greater than 80%. In some embodiments, the compound disclosed here has a human plasma release efficiency of 50%, 55%, 60%, 65%, 70%, 75%, 80%, 85%, 90%, 95%, 96%, 97%, 98%, 99%, or 100%, including all values and ranges therein.

TABLE 1

| Cmpd | | |
|---|---|---|
| No. | Structure | Formula |
| 101 | 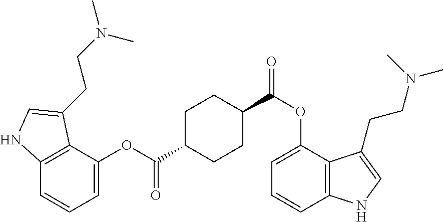 [SMILES: CN(C)CCc1c[nH]c2cccc(OC(=O)[C@H]3CC[C@H](C(=O)Oc4cccc5[nH]cc(CCN(C)C)c45)CC3)c12] | Ia |
| 101a | 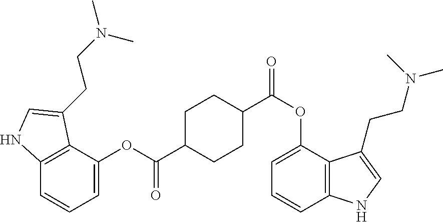 [SMILES: CN(C)CCc1c[nH]c2cccc(OC(=O)C3CCC(C(=O)Oc4cccc5[nH]cc(CCN(C)C)c45)CC3)c12] | Ia |
| 102 | 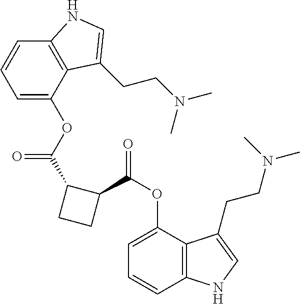 [SMILES: CN(C)CCc1c[nH]c2cccc(OC(=O)[C@H]3CC[C@@H]3C(=O)Oc3cccc4[nH]cc(CCN(C)C)c34)c12] | Ia |
| 102a | 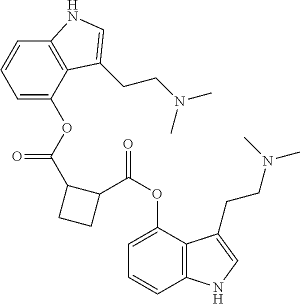 [SMILES: CN(C)CCc1c[nH]c2cccc(OC(=O)C3CCC3C(=O)Oc3cccc4[nH]cc(CCN(C)C)c34)c12] | Ia |
| 103 | 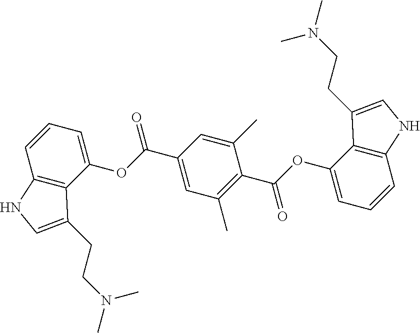 [SMILES: Cc1cc(C(=O)Oc2cccc3[nH]cc(CCN(C)C)c23)cc(C)c1C(=O)Oc1cccc2[nH]cc(CCN(C)C)c12] | Ia |
| 104 | 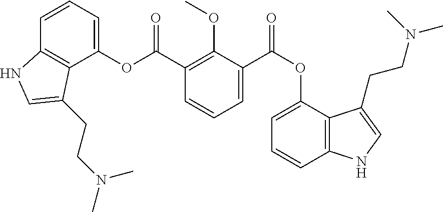 [SMILES: COc1c(C(=O)Oc2cccc3[nH]cc(CCN(C)C)c23)cccc1C(=O)Oc1cccc2[nH]cc(CCN(C)C)c12] | Ia |
| 105 | 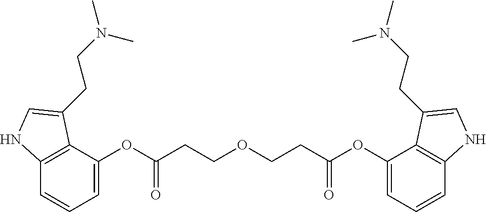 [SMILES: CN(C)CCc1c[nH]c2cccc(OC(=O)CCOCCC(=O)Oc3cccc4[nH]cc(CCN(C)C)c34)c12] | Ia |
| 106 | 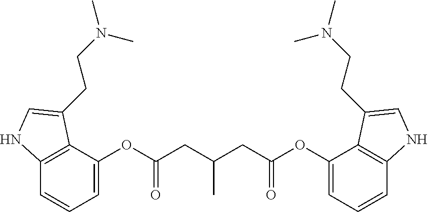 [SMILES: CC(CC(=O)Oc1cccc2[nH]cc(CCN(C)C)c12)CC(=O)Oc1cccc2[nH]cc(CCN(C)C)c12] | Ia |
| 107 | 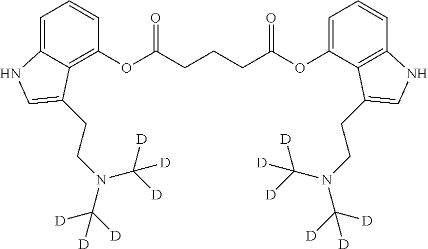 [SMILES: [2H]C([2H])([2H])N(CCc1c[nH]c2cccc(OC(=O)CCCC(=O)Oc3cccc4[nH]cc(CCN(C([2H])([2H])[2H])C([2H])([2H])[2H])c34)c12)C([2H])([2H])[2H]] | Ia |
| 108 | 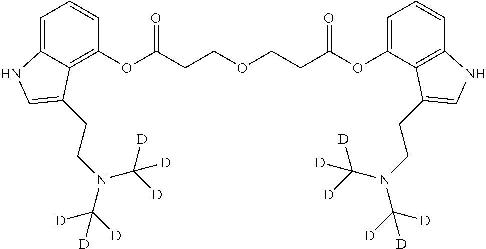 [SMILES: [2H]C([2H])([2H])N(CCc1c[nH]c2cccc(OC(=O)CCOCCC(=O)Oc3cccc4[nH]cc(CCN(C([2H])([2H])[2H])C([2H])([2H])[2H])c34)c12)C([2H])([2H])[2H]] | Ia |
| 109 | 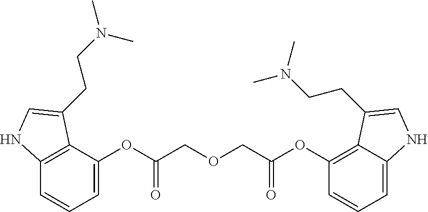 [SMILES: CN(C)CCc1c[nH]c2cccc(OC(=O)COCC(=O)Oc3cccc4[nH]cc(CCN(C)C)c34)c12] | Ia |
| 110 | 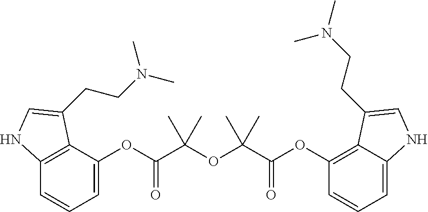 [SMILES: CN(C)CCc1c[nH]c2cccc(OC(=O)C(C)(C)OC(C)(C)C(=O)Oc3cccc4[nH]cc(CCN(C)C)c34)c12] | Ia |
| 112 | 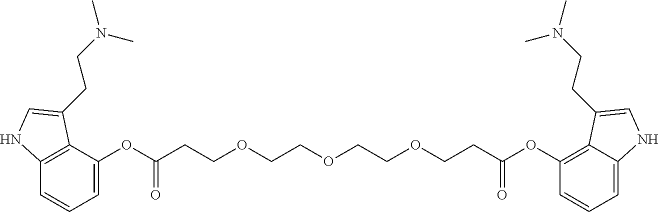 [SMILES: CN(C)CCc1c[nH]c2cccc(OC(=O)CCOCCOCCOCCC(=O)Oc3cccc4[nH]cc(CCN(C)C)c34)c12] | Ia |
| 113 | 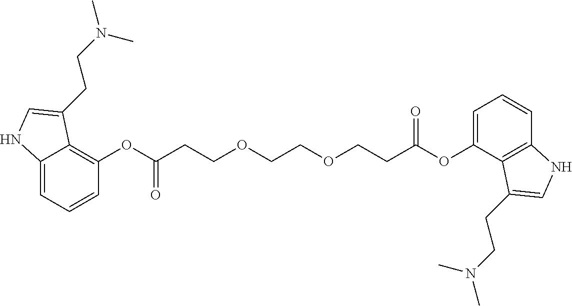 [SMILES: CN(C)CCc1c[nH]c2cccc(OC(=O)CCOCCOCCC(=O)Oc3cccc4[nH]cc(CCN(C)C)c34)c12] | Ia |
| 114 | 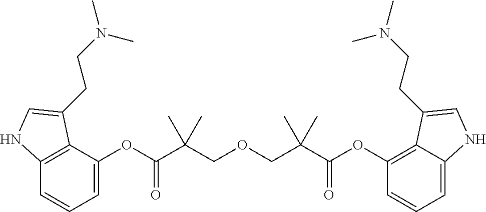 [SMILES: CN(C)CCc1c[nH]c2cccc(OC(=O)C(C)(C)COCC(C)(C)C(=O)Oc3cccc4[nH]cc(CCN(C)C)c34)c12] | Ia |
| 115 | 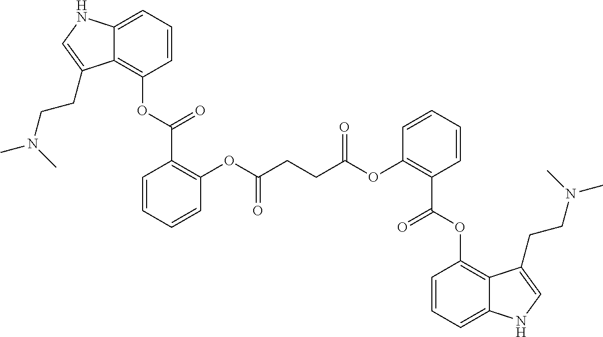 | Ia |
| 116 | 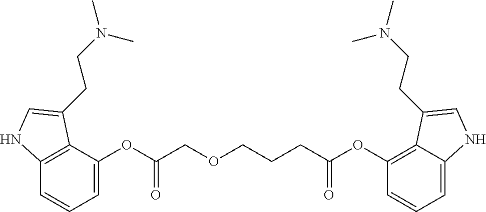 [SMILES: CN(C)CCc1c[nH]c2cccc(OC(=O)CCCOCC(=O)Oc3cccc4[nH]cc(CCN(C)C)c34)c12] | Ia |
| 117 | 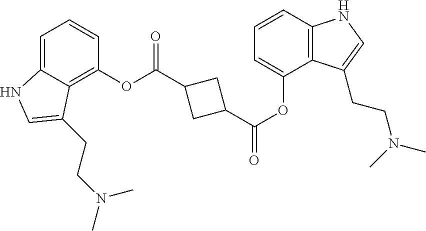 [SMILES: CN(C)CCc1c[nH]c2cccc(OC(=O)C3CC(C(=O)Oc4cccc5[nH]cc(CCN(C)C)c45)C3)c12] | Ia |
| 118 | 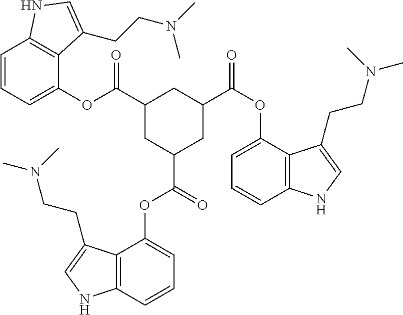 [SMILES: CN(C)CCc1c[nH]c2cccc(OC(=O)C3CC(C(=O)Oc4cccc5[nH]cc(CCN(C)C)c45)CC(C(=O)Oc4cccc5[nH]cc(CCN(C)C)c45)C3)c12] | Ib |
| 118a | 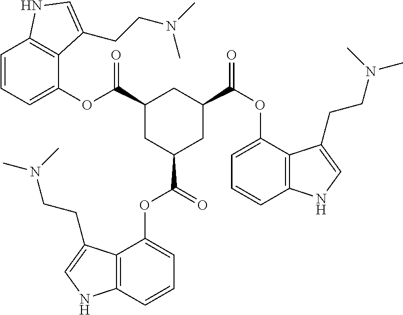 [SMILES: CN(C)CCc1c[nH]c2cccc(OC(=O)[C@H]3C[C@@H](C(=O)Oc4cccc5[nH]cc(CCN(C)C)c45)C[C@@H](C(=O)Oc4cccc5[nH]cc(CCN(C)C)c45)C3)c12] | Ib |
| 119 | 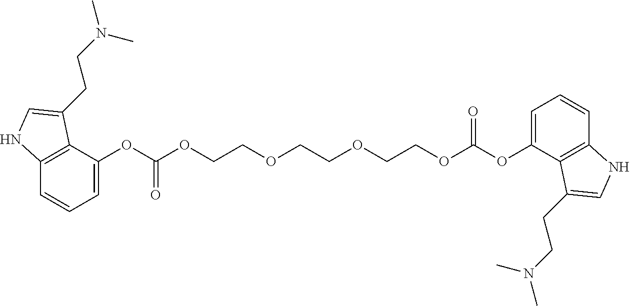 [SMILES: CN(C)CCc1c[nH]c2cccc(OC(=O)OCCOCCOCCOC(=O)Oc3cccc4[nH]cc(CCN(C)C)c34)c12] | iIa |
| 120 | 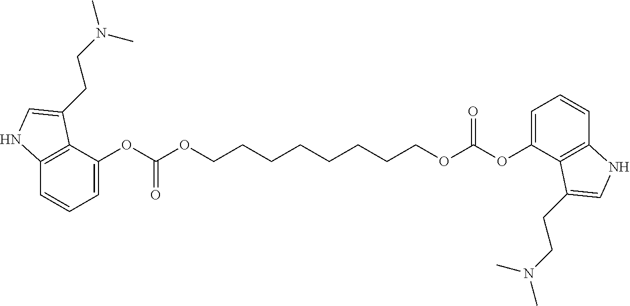 [SMILES: CN(C)CCc1c[nH]c2cccc(OC(=O)OCCCCCCCCOC(=O)Oc3cccc4[nH]cc(CCN(C)C)c34)c12] | iIa |
| 121 | 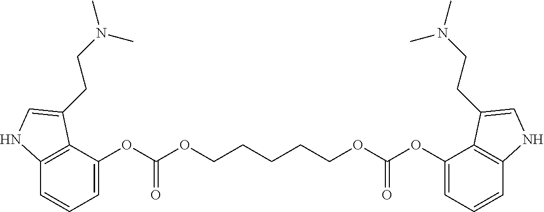 [SMILES: CN(C)CCc1c[nH]c2cccc(OC(=O)OCCCCCOC(=O)Oc3cccc4[nH]cc(CCN(C)C)c34)c12] | iIa |
| 156 | 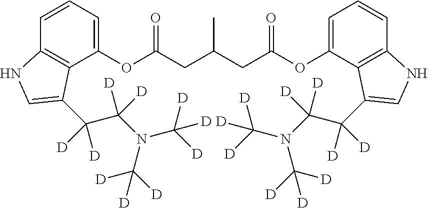 [SMILES: [2H]C([2H])([2H])N(C([2H])([2H])[2H])C([2H])([2H])C([2H])([2H])c1c[nH]c2cccc(OC(=O)CC(C)CC(=O)Oc3cccc4[nH]cc(C([2H])([2H])C([2H])([2H])N(C([2H])([2H])[2H])C([2H])([2H])[2H])c34)c12] | Ia |
| 157 | 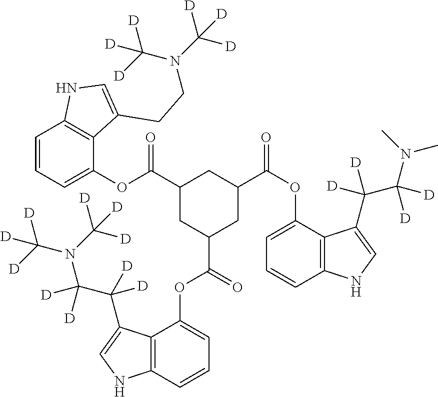 [SMILES: [2H]C([2H])([2H])N(CCc1c[nH]c2cccc(OC(=O)C3CC(C(=O)Oc4cccc5[nH]cc(C([2H])([2H])C([2H])([2H])N(C)C)c45)CC(C(=O)Oc4cccc5[nH]cc(C([2H])([2H])C([2H])([2H])N(C([2H])([2H])[2H])C([2H])([2H])[2H])c45)C3)c12)C([2H])([2H])[2H]] | Ib |
| 159 | 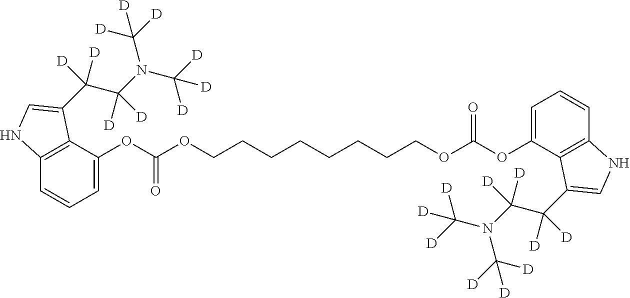 [SMILES: [2H]C([2H])([2H])N(C([2H])([2H])[2H])C([2H])([2H])C([2H])([2H])c1c[nH]c2cccc(OC(=O)OCCCCCCCCOC(=O)Oc3cccc4[nH]cc(C([2H])([2H])C([2H])([2H])N(C([2H])([2H])[2H])C([2H])([2H])[2H])c34)c12] | iIa |
| 160 | 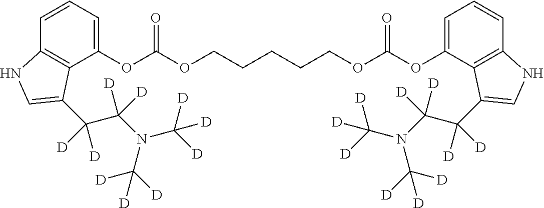 [SMILES: [2H]C([2H])([2H])N(C([2H])([2H])[2H])C([2H])([2H])C([2H])([2H])c1c[nH]c2cccc(OC(=O)OCCCCCOC(=O)Oc3cccc4[nH]cc(C([2H])([2H])C([2H])([2H])N(C([2H])([2H])[2H])C([2H])([2H])[2H])c34)c12] | iIa |
| 222 | 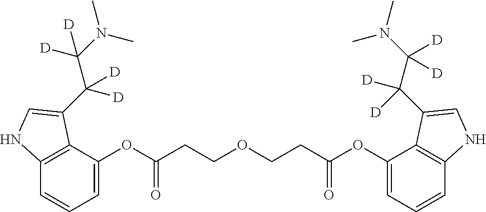 [SMILES: [2H]C([2H])(c1c[nH]c2cccc(OC(=O)CCOCCC(=O)Oc3cccc4[nH]cc(C([2H])([2H])C([2H])([2H])N(C)C)c34)c12)C([2H])([2H])N(C)C] | iIa |

TABLE 2

| Cmpd No. | Structure | Formula |
|---|---|---|
| 130 | 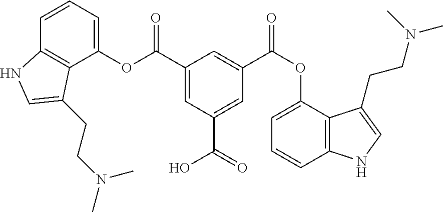 [SMILES: CN(C)CCc1c[nH]c2cccc(OC(=O)c3cc(C(=O)O)cc(C(=O)Oc4cccc5[nH]cc(CCN(C)C)c45)c3)c12] | Ia |
| 131 | 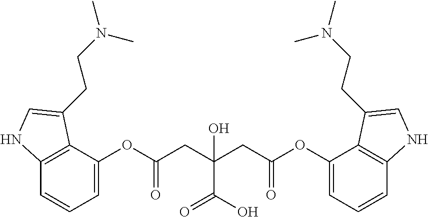 [SMILES: CN(C)CCc1c[nH]c2cccc(OC(=O)CC(O)(CC(=O)Oc3cccc4[nH]cc(CCN(C)C)c34)C(=O)O)c12] | Ia |
| 132 | 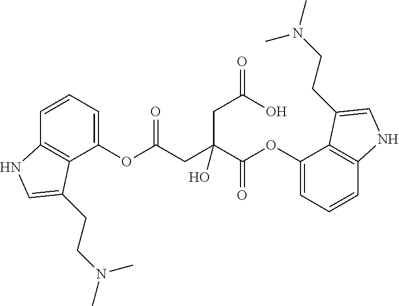 [SMILES: CN(C)CCc1c[nH]c2cccc(OC(=O)CC(O)(CC(=O)O)C(=O)Oc3cccc4[nH]cc(CCN(C)C)c34)c12] | Ia |
| 133 | 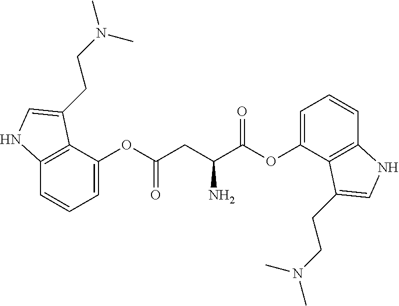 [SMILES: CN(C)CCc1c[nH]c2cccc(OC(=O)C[C@H](N)C(=O)Oc3cccc4[nH]cc(CCN(C)C)c34)c12] | Ia |
| 133a | 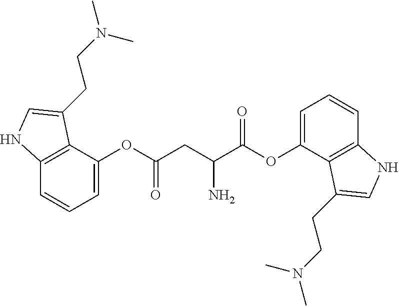 [SMILES: CN(C)CCc1c[nH]c2cccc(OC(=O)CC(N)C(=O)Oc3cccc4[nH]cc(CCN(C)C)c34)c12] | |
| 135 | 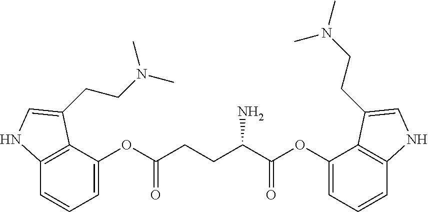 [SMILES: CN(C)CCc1c[nH]c2cccc(OC(=O)CC[C@H](N)C(=O)Oc3cccc4[nH]cc(CCN(C)C)c34)c12] | Ia |
| 136 | 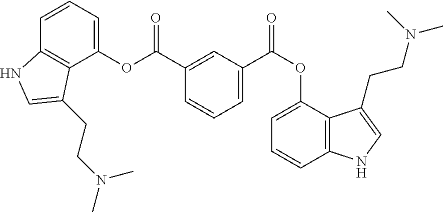 [SMILES: CN(C)CCc1c[nH]c2cccc(OC(=O)c3cccc(C(=O)Oc4cccc5[nH]cc(CCN(C)C)c45)c3)c12] | Ia |
| 138 | 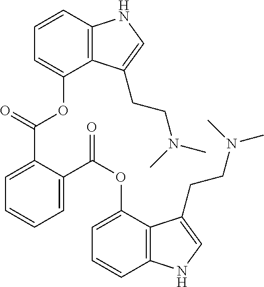 [SMILES: CN(C)CCc1c[nH]c2cccc(OC(=O)c3ccccc3C(=O)Oc3cccc4[nH]cc(CCN(C)C)c34)c12] | Ia |
| 139 | 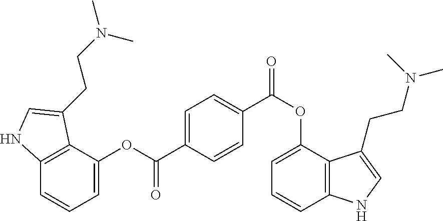 [SMILES: CN(C)CCc1c[nH]c2cccc(OC(=O)c3ccc(C(=O)Oc4cccc5[nH]cc(CCN(C)C)c45)cc3)c12] | Ia |
| 140 | 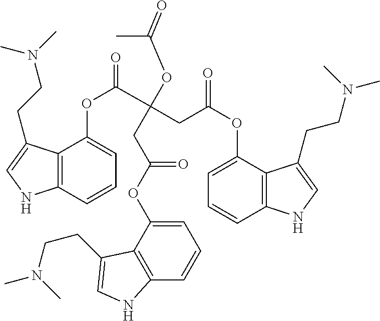 | Ia |
| 141 | 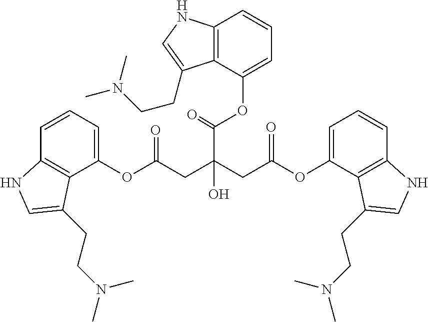 [SMILES: CN(C)CCc1c[nH]c2cccc(OC(=O)CC(O)(CC(=O)Oc3cccc4[nH]cc(CCN(C)C)c34)C(=O)Oc3cccc4[nH]cc(CCN(C)C)c34)c12] | Ib |
| 142 | 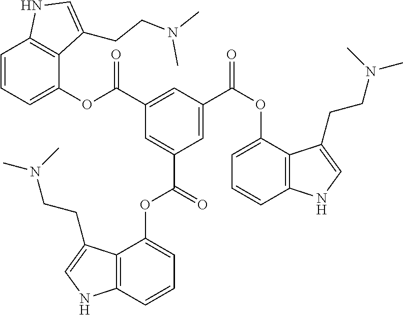 [SMILES: CN(C)CCc1c[nH]c2cccc(OC(=O)c3cc(C(=O)Oc4cccc5[nH]cc(CCN(C)C)c45)cc(C(=O)Oc4cccc5[nH]cc(CCN(C)C)c45)c3)c12] | Ib |
| 143 | 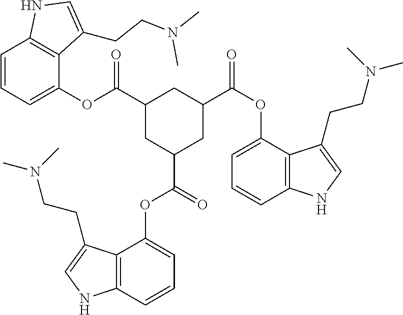 [SMILES: CN(C)CCc1c[nH]c2cccc(OC(=O)C3CC(C(=O)Oc4cccc5[nH]cc(CCN(C)C)c45)CC(C(=O)Oc4cccc5[nH]cc(CCN(C)C)c45)C3)c12] | Ib |
| 144 | 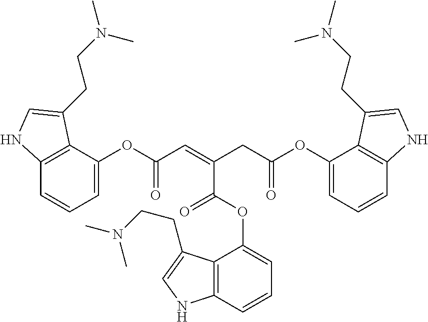 [SMILES: CN(C)CCc1c[nH]c2cccc(OC(=O)/C=C(/CC(=O)Oc3cccc4[nH]cc(CCN(C)C)c34)C(=O)Oc3cccc4[nH]cc(CCN(C)C)c34)c12] | Ib |
| 145 | 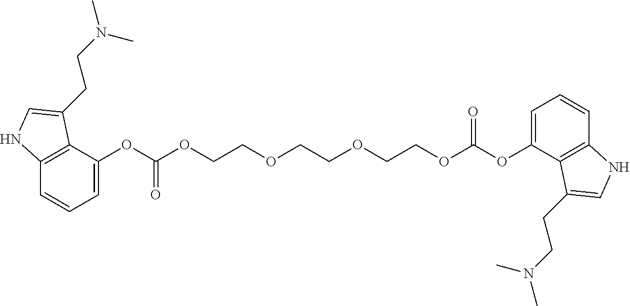 [SMILES: CN(C)CCc1c[nH]c2cccc(OC(=O)OCCOCCOCCOC(=O)Oc3cccc4[nH]cc(CCN(C)C)c34)c12] | iIa |
| 147 | 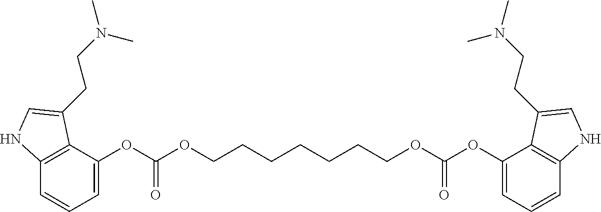 [SMILES: CN(C)CCc1c[nH]c2cccc(OC(=O)OCCCCCCCOC(=O)Oc3cccc4[nH]cc(CCN(C)C)c34)c12] | iIa |
| 148 | 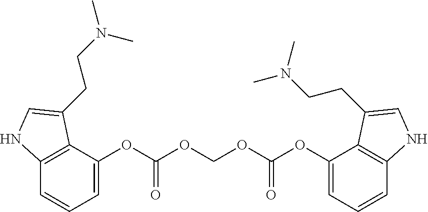 [SMILES: CN(C)CCc1c[nH]c2cccc(OC(=O)OCOC(=O)Oc3cccc4[nH]cc(CCN(C)C)c34)c12] | iIa |
| 149 | 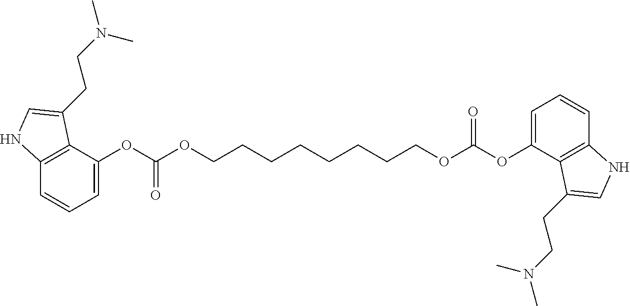 [SMILES: CN(C)CCc1c[nH]c2cccc(OC(=O)OCCCCCCCCOC(=O)Oc3cccc4[nH]cc(CCN(C)C)c34)c12] | iIa |
| 150 | 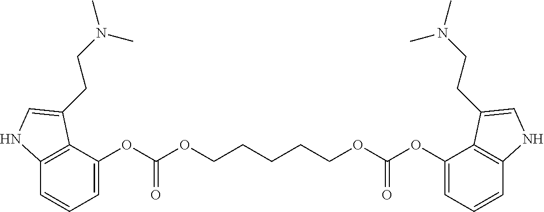 [SMILES: CN(C)CCc1c[nH]c2cccc(OC(=O)OCCCCCOC(=O)Oc3cccc4[nH]cc(CCN(C)C)c34)c12] | iIa |
| 152 | 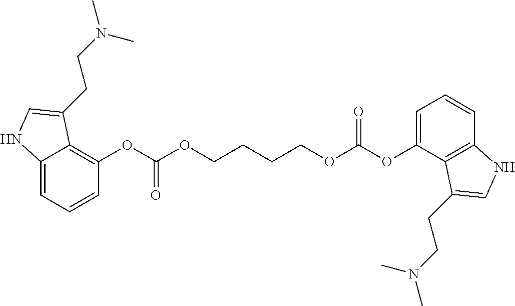 [SMILES: CN(C)CCc1c[nH]c2cccc(OC(=O)OCCCCOC(=O)Oc3cccc4[nH]cc(CCN(C)C)c34)c12] | iIa |
| 153 | 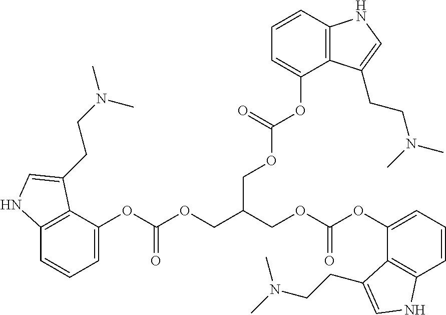 [SMILES: CN(C)CCc1c[nH]c2cccc(OC(=O)OCC(COC(=O)Oc3cccc4[nH]cc(CCN(C)C)c34)COC(=O)Oc3cccc4[nH]cc(CCN(C)C)c34)c12] | iIb |
| 154 | 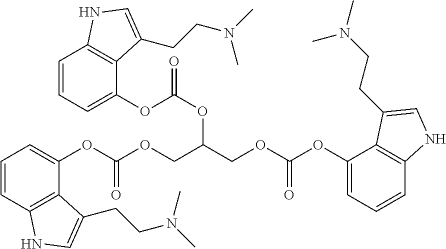 [SMILES: CN(C)CCc1c[nH]c2cccc(OC(=O)OCC(COC(=O)Oc3cccc4[nH]cc(CCN(C)C)c34)OC(=O)Oc3cccc4[nH]cc(CCN(C)C)c34)c12] | iIb |
| 155 | 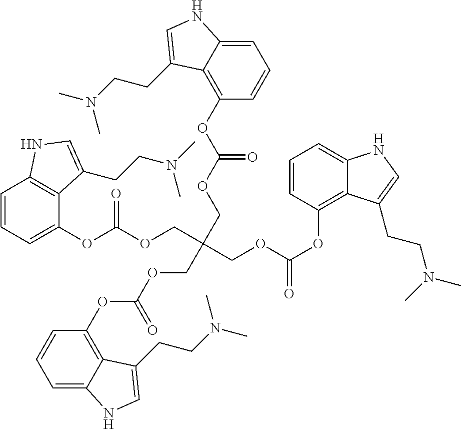 [SMILES: CN(C)CCc1c[nH]c2cccc(OC(=O)OCC(COC(=O)Oc3cccc4[nH]cc(CCN(C)C)c34)(COC(=O)Oc3cccc4[nH]cc(CCN(C)C)c34)COC(=O)Oc3cccc4[nH]cc(CCN(C)C)c34)c12] | IIc |

TABLE 3

| Cmpd No. | Structure | Formula |
|---|---|---|
| 122 | 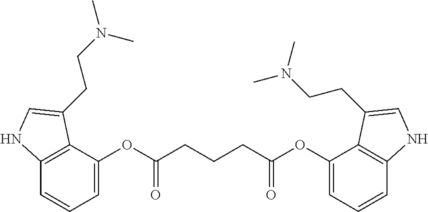 [SMILES: CN(C)CCc1c[nH]c2cccc(OC(=O)CCCC(=O)Oc3cccc4[nH]cc(CCN(C)C)c34)c12] | Ia |
| 123 | 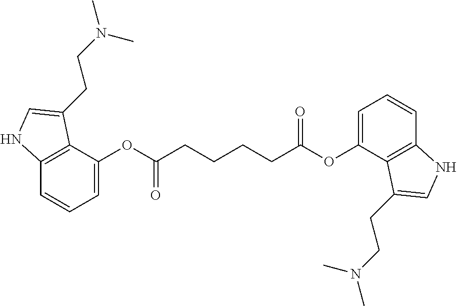 [SMILES: CN(C)CCc1c[nH]c2cccc(OC(=O)CCCCC(=O)Oc3cccc4[nH]cc(CCN(C)C)c34)c12] | Ia |
| 124 | 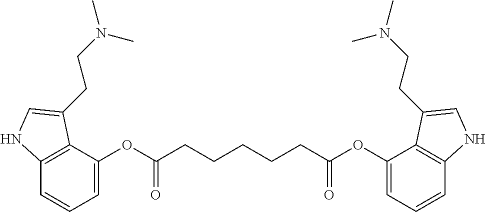 [SMILES: CN(C)CCc1c[nH]c2cccc(OC(=O)CCCCCC(=O)Oc3cccc4[nH]cc(CCN(C)C)c34)c12] | Ia |
| 134 | 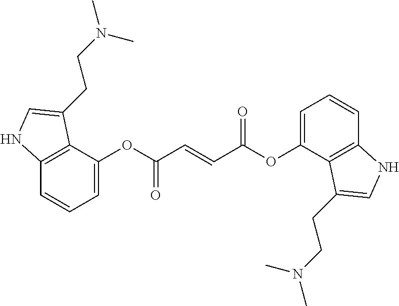 [SMILES: CN(C)CCc1c[nH]c2cccc(OC(=O)/C=C/C(=O)Oc3cccc4[nH]cc(CCN(C)C)c34)c12] | Ia |
| 137 | 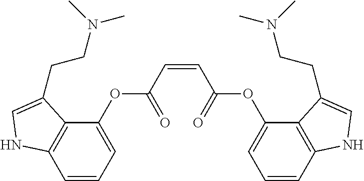 [SMILES: CN(C)CCc1c[nH]c2cccc(OC(=O)/C=C\C(=O)Oc3cccc4[nH]cc(CCN(C)C)c34)c12] | Ia |
| 156 | 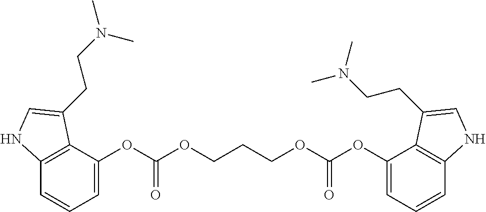 [SMILES: CN(C)CCc1c[nH]c2cccc(OC(=O)OCCCOC(=O)Oc3cccc4[nH]cc(CCN(C)C)c34)c12] | iIa |
| 157 | 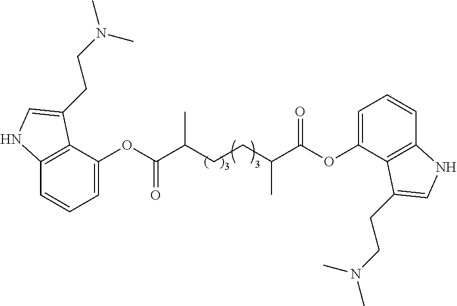 | Ia |
| 158 | 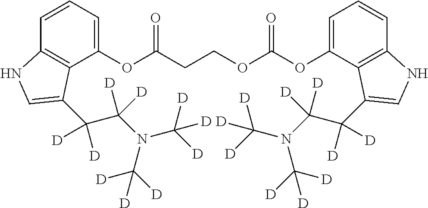 [SMILES: [2H]C([2H])([2H])N(C([2H])([2H])[2H])C([2H])([2H])C([2H])([2H])c1c[nH]c2cccc(OC(=O)CCOC(=O)Oc3cccc4[nH]cc(C([2H])([2H])C([2H])([2H])N(C([2H])([2H])[2H])C([2H])([2H])[2H])c34)c12] | Ia |
| 146 | 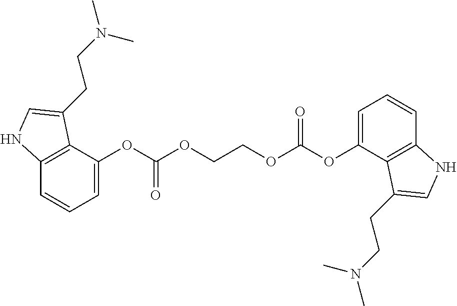 [SMILES: CN(C)CCc1c[nH]c2cccc(OC(=O)OCCOC(=O)Oc3cccc4[nH]cc(CCN(C)C)c34)c12] | iIa |
| 151 | 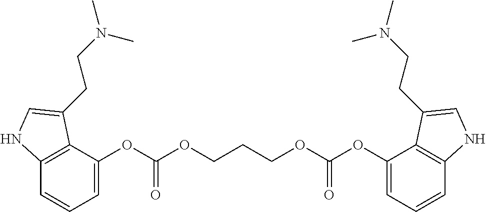 [SMILES: CN(C)CCc1c[nH]c2cccc(OC(=O)OCCCOC(=O)Oc3cccc4[nH]cc(CCN(C)C)c34)c12] | iIa |

TABLE 4

| Cpd No. | Structure | Formula |
|---|---|---|
| 161 | 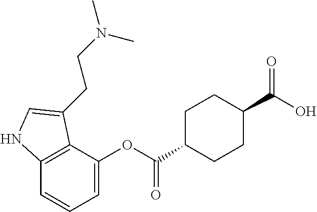 [SMILES: CN(C)CCc1c[nH]c2cccc(OC(=O)[C@H]3CC[C@H](C(=O)O)CC3)c12] | III |
| 162 | 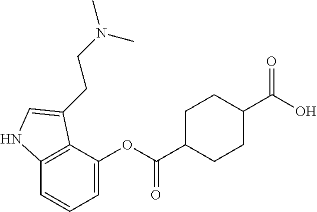 [SMILES: CN(C)CCc1c[nH]c2cccc(OC(=O)C3CCC(C(=O)O)CC3)c12] | III |
| 163 | 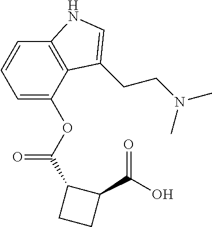 [SMILES: CN(C)CCc1c[nH]c2cccc(OC(=O)[C@H]3CC[C@@H]3C(=O)O)c12] | III |
| 164 | 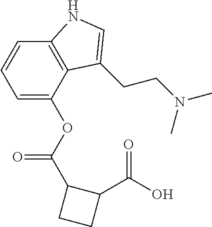 [SMILES: CN(C)CCc1c[nH]c2cccc(OC(=O)C3CCC3C(=O)O)c12] | III |
| 165 | 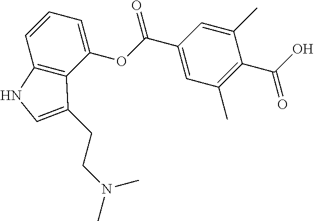 [SMILES: Cc1cc(C(=O)Oc2cccc3[nH]cc(CCN(C)C)c23)cc(C)c1C(=O)O] | III |
| 166 | 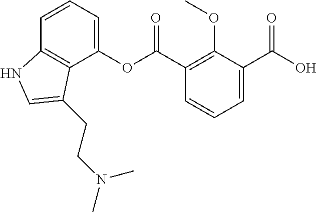 [SMILES: COc1c(C(=O)O)cccc1C(=O)Oc1cccc2[nH]cc(CCN(C)C)c12] | III |
| 167 | 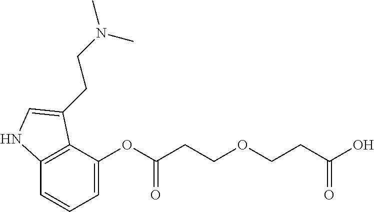 [SMILES: CN(C)CCc1c[nH]c2cccc(OC(=O)CCOCCC(=O)O)c12] | III |
| 168 | 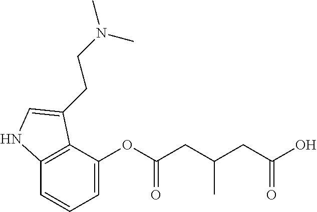 [SMILES: CC(CC(=O)O)CC(=O)Oc1cccc2[nH]cc(CCN(C)C)c12] | III |
| 169 | 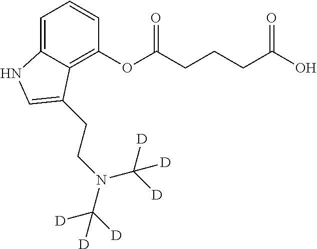 [SMILES: [2H]C([2H])([2H])N(CCc1c[nH]c2cccc(OC(=O)CCCC(=O)O)c12)C([2H])([2H])[2H]] | III |
| 170 | 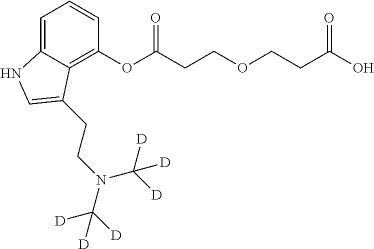 [SMILES: [2H]C([2H])([2H])N(CCc1c[nH]c2cccc(OC(=O)CCOCCC(=O)O)c12)C([2H])([2H])[2H]] | III |
| 171 | 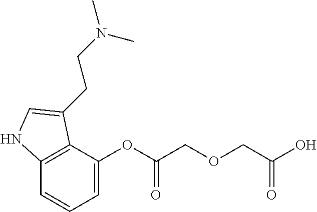 [SMILES: CN(C)CCc1c[nH]c2cccc(OC(=O)COCC(=O)O)c12] | III |
| 172 | 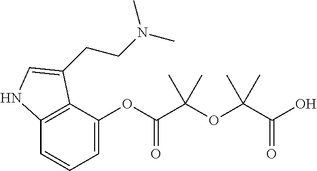 [SMILES: CN(C)CCc1c[nH]c2cccc(OC(=O)C(C)(C)OC(C)(C)C(=O)O)c12] | III |
| 173 | 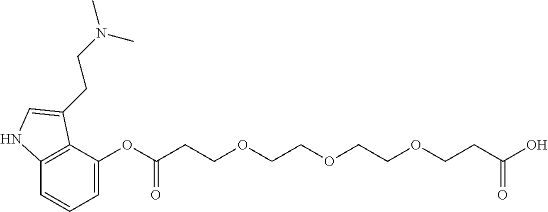 [SMILES: CN(C)CCc1c[nH]c2cccc(OC(=O)CCOCCOCCOCCC(=O)O)c12] | III |
| 174 | 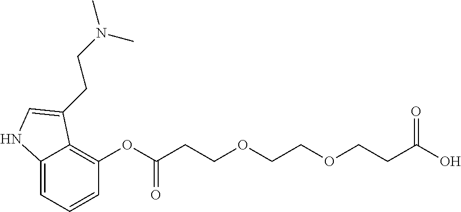 [SMILES: CN(C)CCc1c[nH]c2cccc(OC(=O)CCOCCOCCC(=O)O)c12] | III |
| 175 | 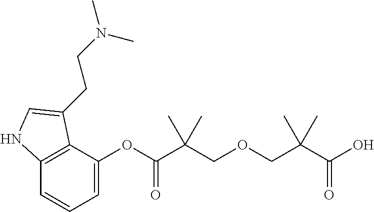 [SMILES: CN(C)CCc1c[nH]c2cccc(OC(=O)C(C)(C)COCC(C)(C)C(=O)O)c12] | III |
| 176 | 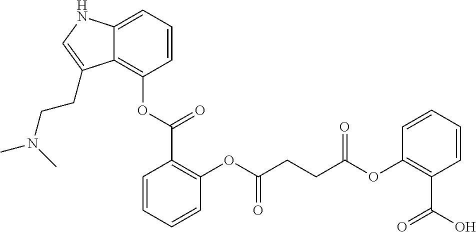 [SMILES: CN(C)CCc1c[nH]c2cccc(OC(=O)c3ccccc3OC(=O)CCC(=O)Oc3ccccc3C(=O)O)c12] | III |
| 177 | 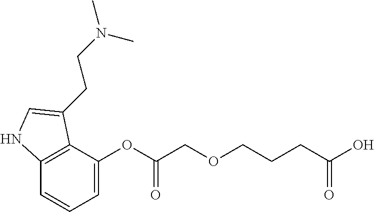 [SMILES: CN(C)CCc1c[nH]c2cccc(OC(=O)COCCCC(=O)O)c12] | III |
| 178 | 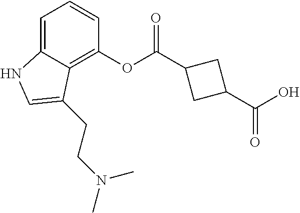 [SMILES: CN(C)CCc1c[nH]c2cccc(OC(=O)C3CC(C(=O)O)C3)c12] | III |
| 179 | 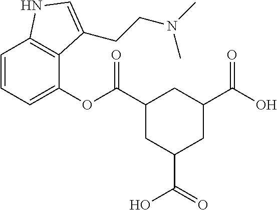 [SMILES: CN(C)CCc1c[nH]c2cccc(OC(=O)C3CC(C(=O)O)CC(C(=O)O)C3)c12] | III |
| 180 | 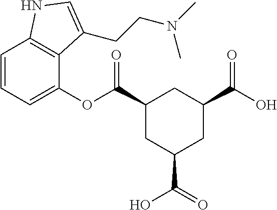 [SMILES: CN(C)CCc1c[nH]c2cccc(OC(=O)[C@@H]3C[C@H](C(=O)O)C[C@H](C(=O)O)C3)c12] | III |
| 181 | 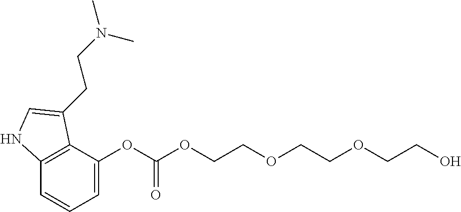 [SMILES: CN(C)CCc1c[nH]c2cccc(OC(=O)OCCOCCOCCO)c12] | III |
| 182 | 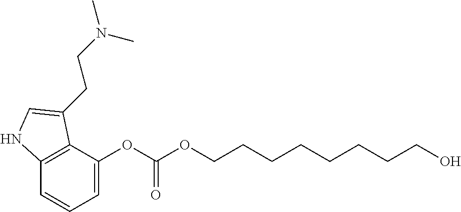 [SMILES: CN(C)CCc1c[nH]c2cccc(OC(=O)OCCCCCCCCO)c12] | III |
| 183 | 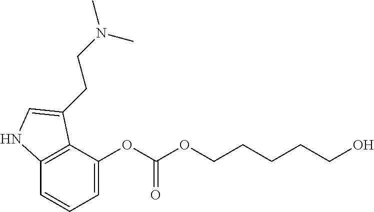 [SMILES: CN(C)CCc1c[nH]c2cccc(OC(=O)OCCCCCO)c12] | III |
| 184 | 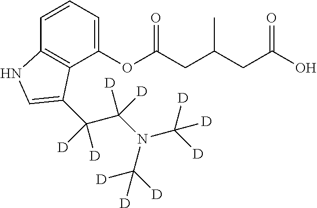 [SMILES: [2H]C([2H])([2H])N(C([2H])([2H])[2H])C([2H])([2H])C([2H])([2H])c1c[nH]c2cccc(OC(=O)CC(C)CC(=O)O)c12] | III |
| 185 | 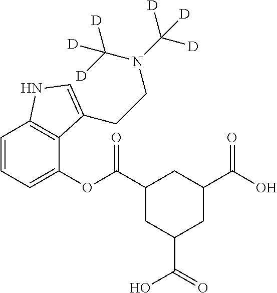 [SMILES: [2H]C([2H])([2H])N(CCc1c[nH]c2cccc(OC(=O)C3CC(C(=O)O)CC(C(=O)O)C3)c12)C([2H])([2H])[2H]] | III |
| 186 | 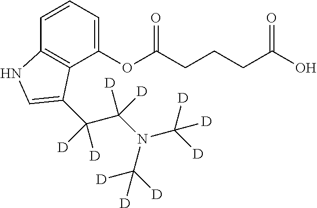 [SMILES: [2H]C([2H])([2H])N(C([2H])([2H])[2H])C([2H])([2H])C([2H])([2H])c1c[nH]c2cccc(OC(=O)CCCC(=O)O)c12] | III |
| 187 | 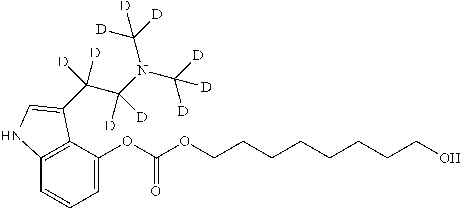 [SMILES: [2H]C([2H])([2H])N(C([2H])([2H])[2H])C([2H])([2H])C([2H])([2H])c1c[nH]c2cccc(OC(=O)OCCCCCCCCO)c12] | III |
| 188 | 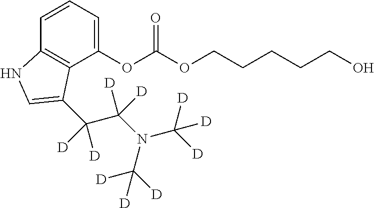 [SMILES: [2H]C([2H])([2H])N(C([2H])([2H])[2H])C([2H])([2H])C([2H])([2H])c1c[nH]c2cccc(OC(=O)OCCCCCO)c12] | III |
| 189 | 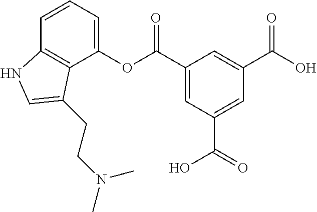 [SMILES: CN(C)CCc1c[nH]c2cccc(OC(=O)c3cc(C(=O)O)cc(C(=O)O)c3)c12] | III |
| 190 | 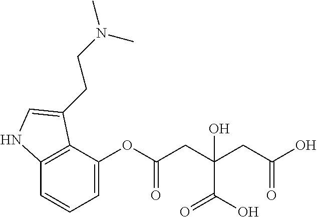 [SMILES: CN(C)CCc1c[nH]c2cccc(OC(=O)CC(O)(CC(=O)O)C(=O)O)c12] | III |
| 191 | 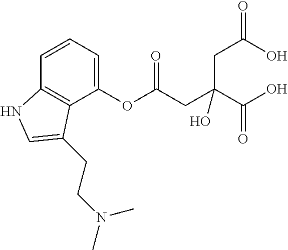 [SMILES: CN(C)CCc1c[nH]c2cccc(OC(=O)CC(O)(CC(=O)O)C(=O)O)c12] | III |
| 192 | 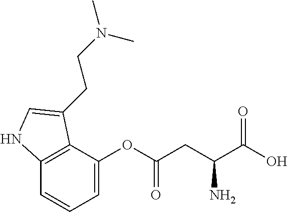 [SMILES: CN(C)CCc1c[nH]c2cccc(OC(=O)C[C@H](N)C(=O)O)c12] | III |
| 193 | 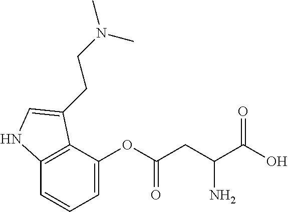 [SMILES: CN(C)CCc1c[nH]c2cccc(OC(=O)CC(N)C(=O)O)c12] | III |
| 194 | 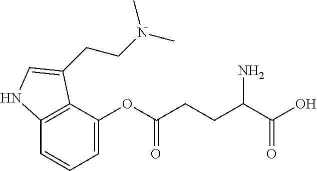 [SMILES: CN(C)CCc1c[nH]c2cccc(OC(=O)CCC(N)C(=O)O)c12] | III |
| 195 | 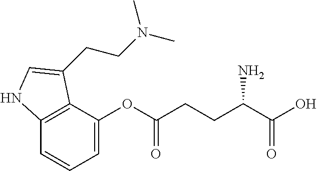 [SMILES: CN(C)CCc1c[nH]c2cccc(OC(=O)CC[C@H](N)C(=O)O)c12] | III |
| 196 | 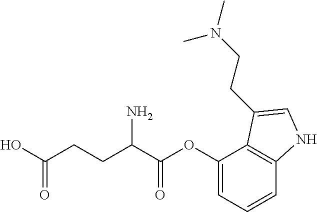 [SMILES: CN(C)CCc1c[nH]c2cccc(OC(=O)C(N)CCC(=O)O)c12] | III |
| 197 | 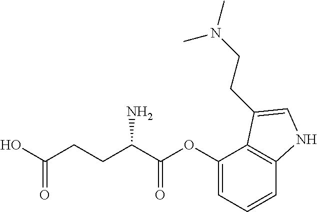 [SMILES: CN(C)CCc1c[nH]c2cccc(OC(=O)[C@@H](N)CCC(=O)O)c12] | III |
| 198 | 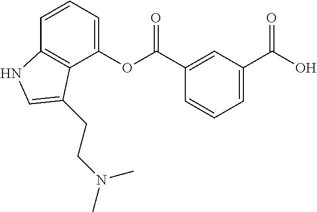 [SMILES: CN(C)CCc1c[nH]c2cccc(OC(=O)c3cccc(C(=O)O)c3)c12] | III |
| 199 | 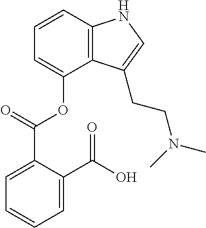 [SMILES: CN(C)CCc1c[nH]c2cccc(OC(=O)c3ccccc3C(=O)O)c12] | III |
| 200 | 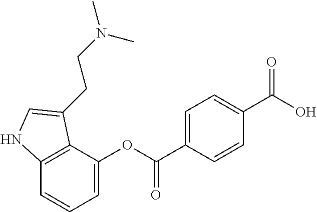 [SMILES: CN(C)CCc1c[nH]c2cccc(OC(=O)c3ccc(C(=O)O)cc3)c12] | III |
| 201 | 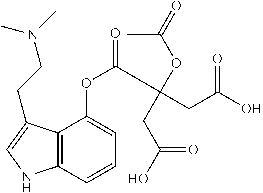 [SMILES: CC(=O)OC(CC(=O)O)(CC(=O)O)C(=O)Oc1cccc2[nH]cc(CCN(C)C)c12] | III |
| 202 | 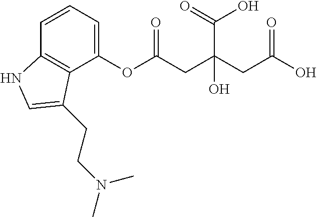 [SMILES: CN(C)CCc1c[nH]c2cccc(OC(=O)CC(O)(CC(=O)O)C(=O)O)c12] | III |
| 203 | 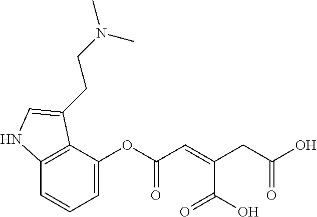 [SMILES: CN(C)CCc1c[nH]c2cccc(OC(=O)/C=C(/CC(=O)O)C(=O)O)c12] | III |
| 204 | 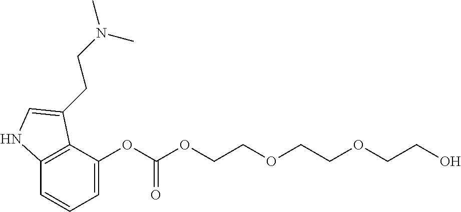 [SMILES: CN(C)CCc1c[nH]c2cccc(OC(=O)OCCOCCOCCO)c12] | III |
| 205 | 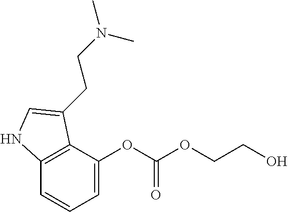 [SMILES: CN(C)CCc1c[nH]c2cccc(OC(=O)OCCO)c12] | III |
| 206 | 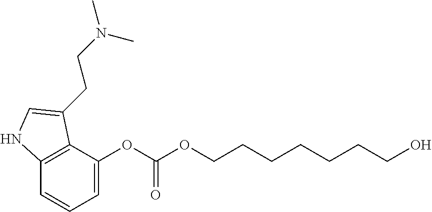 [SMILES: CN(C)CCc1c[nH]c2cccc(OC(=O)OCCCCCCCO)c12] | III |
| 207 | 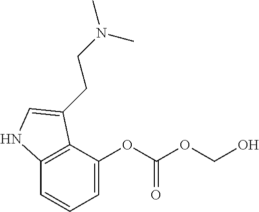 [SMILES: CN(C)CCc1c[nH]c2cccc(OC(=O)OCO)c12] | III |
| 208 | 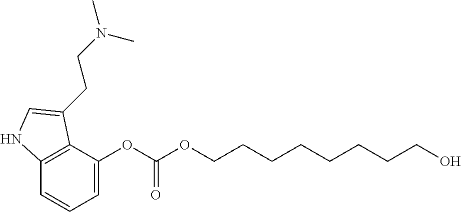 [SMILES: CN(C)CCc1c[nH]c2cccc(OC(=O)OCCCCCCCCO)c12] | III |
| 209 | 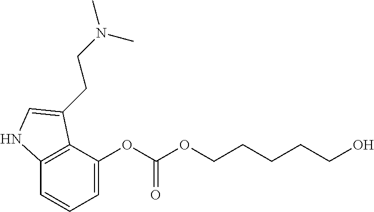 [SMILES: CN(C)CCc1c[nH]c2cccc(OC(=O)OCCCCCO)c12] | III |
| 210 | 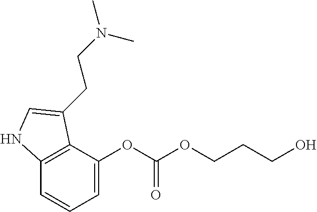 [SMILES: CN(C)CCc1c[nH]c2cccc(OC(=O)OCCCO)c12] | III |
| 211 | 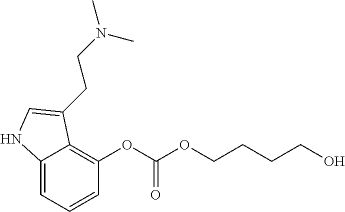 [SMILES: CN(C)CCc1c[nH]c2cccc(OC(=O)OCCCCO)c12] | III |
| 212 | 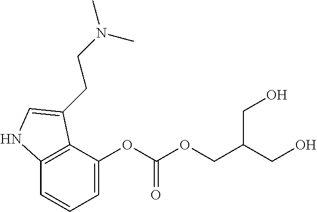 [SMILES: CN(C)CCc1c[nH]c2cccc(OC(=O)OCC(CO)CO)c12] | III |
| 213 | 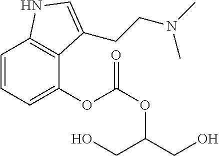 [SMILES: CN(C)CCc1c[nH]c2cccc(OC(=O)OC(CO)CO)c12] | III |
| 214 | 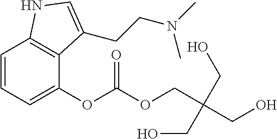 [SMILES: CN(C)CCc1c[nH]c2cccc(OC(=O)OCC(CO)(CO)CO)c12] | III |
| 215 | 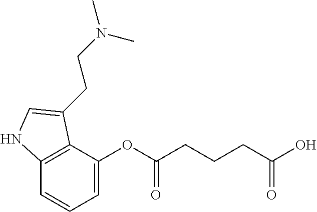 [SMILES: CN(C)CCc1c[nH]c2cccc(OC(=O)CCCC(=O)O)c12] | III |
| 216 | 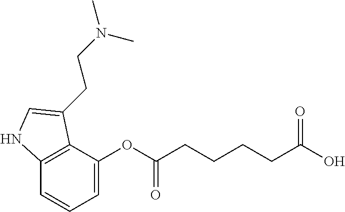 [SMILES: CN(C)CCc1c[nH]c2cccc(OC(=O)CCCCC(=O)O)c12] | III |
| 217 | 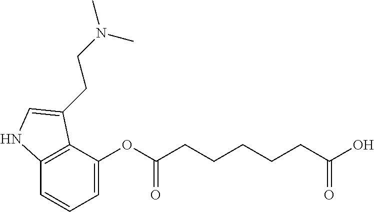 [SMILES: CN(C)CCc1c[nH]c2cccc(OC(=O)CCCCCC(=O)O)c12] | III |
| 218 | 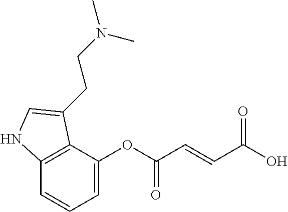 [SMILES: CN(C)CCc1c[nH]c2cccc(OC(=O)/C=C/C(=O)O)c12] | III |
| 219 | 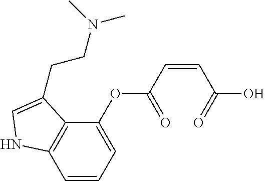 [SMILES: CN(C)CCc1c[nH]c2cccc(OC(=O)/C=C\C(=O)O)c12] | III |
| 220 | 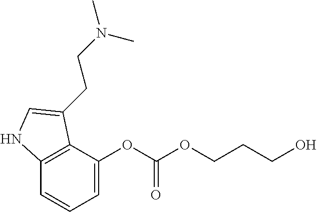 [SMILES: CN(C)CCc1c[nH]c2cccc(OC(=O)OCCCO)c12] | III |
| 221 | 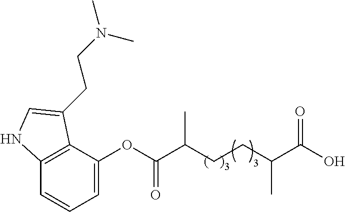 | III |

### Pharmaceutical Compositions

The present disclosure provides pharmaceutical compositions comprising at least one compound disclosed herein and one or more pharmaceutically acceptable excipients.

The compounds provided herein may be administered as compounds per se or may be formulated as pharmaceutical compositions. The pharmaceutical compositions may comprise one or more pharmaceutically acceptable excipients, such as carriers, diluents, fillers, disintegrants, lubricating agents, binders, colorants, pigments, stabilizers, preservatives, and/or antioxidants.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

### Methods of Treatment

The disclosure further relates to compounds disclosed herein, or a pharmaceutical composition comprising at least one compound disclosed here, for use in the treatment of a serotonin 5-HT2A receptor associated disease/disorder. In embodiments, the compounds may be used in the treatment of an anxiety disorder, attention deficit hyperactivity disorder (ADHD), depression (including treatment resistant depression), cluster headache, diminished drive, burn-out, bore-out, migraine, Parkinson's disease, schizophrenia, an eating disorder (including anorexia nervosa), psychotic disorder, schizophrenia, schizophreniform disorder, schizoaffective disorder, bipolar I disorder, bipolar II disorder, major depressive disorder, psychotic depression, delusional disorders, shared psychotic disorder, Shared paranoia disorder, brief psychotic disorder, paranoid personality disorder, schizoid personality disorder, schizotypal personality disorder, social anxiety disorder, substance-induced anxiety disorder, selective mutism, panic disorder, panic attacks, agoraphobia, posttraumatic stress disorder (PTSD), premenstrual dysphoric disorder (PMDD), and premenstrual syndrome (PMS).

In some embodiments, one or more of the compounds of formula (I), (Ia), (Ib), (II), (iIa), (iIb), (IIc), (III), (D-I), (D-Ia), (D-Ib), (D-II), (D-iIa), (D-iIb), (D-IIc) or (D-III), or a deuterated form or pharmaceutically acceptable salt thereof is administered to the subject to treat a disease disclosed herein.

In some embodiments, one or more of the compounds of Table 1, or a deuterated form or pharmaceutically acceptable salt thereof is administered to the subject to treat a disease disclosed herein.

In some embodiments, one or more of the compounds of Table 2, or a deuterated form or pharmaceutically acceptable salt thereof is administered to the subject to treat a disease disclosed herein.

In some embodiments, one or more of the compounds of Table 3, or a deuterated form or pharmaceutically acceptable salt thereof is administered to the subject to treat a disease disclosed herein.

In some embodiments, one or more of the compounds of Table 4, or a deuterated form or pharmaceutically acceptable salt thereof is administered to the subject to treat a disease disclosed herein.

### Numbered Embodiments

1. A compound of formula (I),

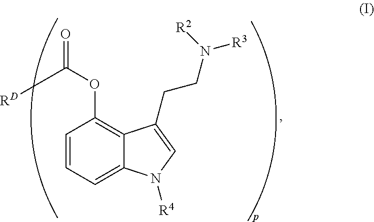

or a pharmaceutically acceptable salt thereof or deuterated form thereof, wherein:
   R² and R³ are independently alkyl;
   R⁴ is H or C(═O)Oalkyl;
   p is 2 or 3;
   R^{D} is a divalent or trivalent group selected from alkylene, alkenylene, alkylene-O-alkylene, —(CH₂)ₙ—(OCH₂CH₂)ₘ—, cycloalkylene, or arylene, or

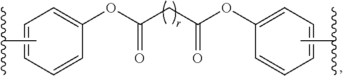

each of which is optionally substituted with 1-4 groups selected from halogen, OH, Oalkyl, alkyl, NH₂, NH(alkyl), N(alkyl)₂, C(═O)OH, C(═O)Oalkyl, OC(═O)alkyl, or C(═O)alkyl;
n is 1, 2, 3, 4, 5, 6, 7 or 8;
m is 1, 2, 3, 4, 5, 6, 7 or 8;
r is 1, 2 or 3;
provided that when R² and R³ are each non-deuterated alkyl, then R^{D} is not (CH₂)₂, (CH₂)₃, (CH₂)₄, (CH₂)₅, (CH₂)₈,

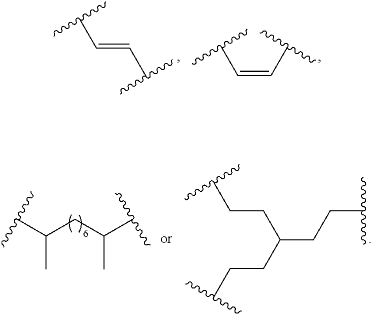

2. The compound of embodiment 1, wherein R^{D} is a divalent or trivalent group selected from C₁₋₁₂ alkylene, C₁₋₆alkylene-O—C₁₋₆alkylene, —(CH₂)ₙ—(OCH₂CH₂)ₘ—, C₃₋₈cycloalkylene, arylene or

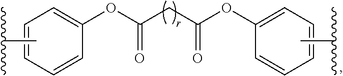

each of which is optionally substituted with 1-4 groups selected from with C₁₋₃ alkyl, OH, OCH₃, NH₂, COOH, or C(═O)alkyl.
3. The compound of embodiment 1, wherein the compound is a compound of formula (Ia)

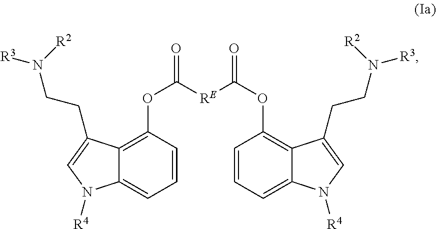

or a pharmaceutically acceptable salt or deuterated thereof,
wherein:
   each R² and R³ are independently alkyl;
   each R⁴ is independently H or C(═O)Oalkyl;
   R^{E} is a divalent group selected from alkylene, cycloalkylene, arylene, alkylene-O-alkylene, —(CH₂)ₙ—(OCH₂CH₂)ₘ— or

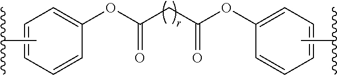

each of which is optionally substituted with one or more groups selected from halogen, alkyl, Oalkyl, NH₂, NHC₁₋₆alkyl, NC(₁₋₆alkyl)₂, COOH, cycloalkyl, and C(═O)Oalkyl;
n is 1, 2, or 3;
m is 1, 2 or 3; and
r is 1, 2 or 3.
4. The compound of embodiment 3, wherein R^{E} is a divalent group selected from C₁₋₁₂ alkylene, C₃₋₈cycloalkylene, arylene, C₁₋₆alkylene-O—C₁₋₆alkylene, —(CH₂)ₙ—(OCH₂CH₂)ₘ— or

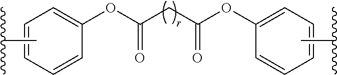

each of which is optionally substituted with one or more groups selected from halogen, C₁₋₃alkyl, Oalkyl, NH₂, NHC₁₋₆alkyl, NC(₁₋₆alkyl)₂, COOH, C₃₋₈ cycloalkyl, and C(═O)Oalkyl.
5. The compound of any one of embodiments 3-4, wherein the alkylene group is linear or branched.
6. The compound of any one of embodiments 3-5, wherein the alkylene group is branched.
7. The compound of any one of embodiments 1-6, wherein R^{D} or R^{E} is

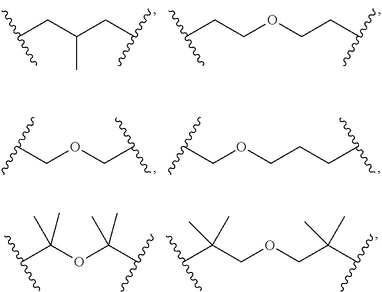

— (CH₂)₂—(OCH₂CH₂)₂—, —(CH₂)₂—(OCH₂CH₂)₃—,

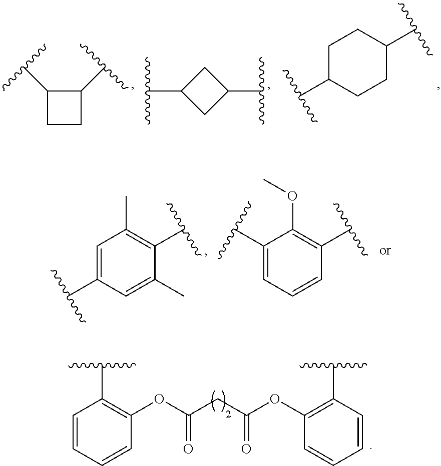

8. The compound of embodiment 1, wherein the compound is a compound of formula (Ib)

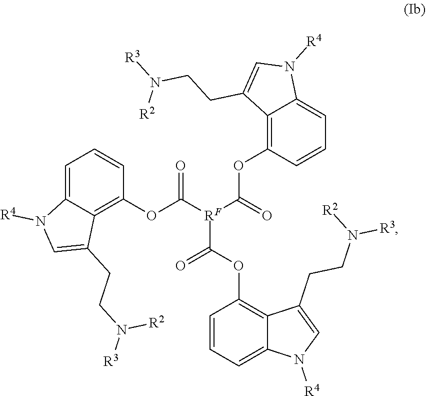

or a pharmaceutically acceptable salt or deuterated form thereof, wherein
each R² and R³ are independently alkyl;
each R⁴ is independently H or C(═O)Oalkyl;
R^{F} is a trivalent cycloalkylene or alkylene group.
9. The compound of embodiment 8, wherein R^{F} is a trivalent C₃₋₈cycloalkylene.
10. The compound of embodiment 8 or 9, wherein R^{F} is

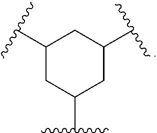 [SMILES: *C1CC(*)CC(*)C1]

11. A compound of formula (iIa),

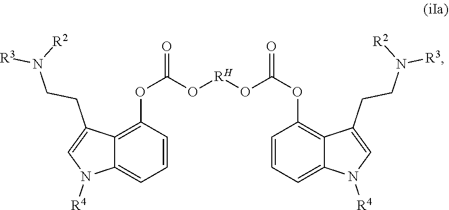

or a pharmaceutically acceptable salt or deuterated form thereof,
wherein:
   each R² and R³ are independently alkyl;
   each R⁴ is independently H or C(═O)Oalkyl;
   R^{H} is —(CH₂)ₙ—(OCH₂CH₂)ₘ— or alkylene;
   n is 1, 2, 3, 4, 5, 6, 7, 8, 9, 10, 11 or 12, and
   m is 1, 2 or 3;
provided that when q is 2 and R² and R³ are each CH₃, R^{H} is not —(CH₂)₃—.
12. The compound of embodiment 11, wherein,
   n is 1, 2, 3, 4, 5, 6, 7, 8, and
   m is 1, 2 or 3.
13. The compound of embodiment 11 or 12, wherein R^{H} is —(CH₂)₅—, —(CH₂)₈— or —(CH₂)₂—(OCH₂CH₂)₂.
14. The compound of any one of embodiments 1-13, wherein R² and R³ are independently C₁₋₆ alkyl.
15. The compound of any one of embodiments 1-14, wherein R² and R³ are —CH₃.
16. The compound of any one of embodiments 1-15, wherein R⁴ is H or C(═O)OC₁₋₆ alkyl.
17. The compound of any one of embodiments 1-16, wherein R⁴ is H or C(═O)OCH₃.
18. The compound of any one of embodiments 1-17, wherein R⁴ is H.
19. The compound of any one of embodiments 1-18, wherein R² and R³ are independently CH₃, and R⁴ is H.
20. A compound selected from Table 1, a pharmaceutically acceptable salt thereof, or a deuterated form thereof.
21. A pharmaceutical composition comprising a compound of embodiment 1-20, or a pharmaceutically acceptable salt thereof.
22. A method of treating a disease comprising administrating a pharmaceutical composition of embodiment 21.
23. A method of treating a disease comprising subcutaneously administrating a pharmaceutical composition of embodiment 21.
24. A method of treating a disease comprising subcutaneously administrating a pharmaceutical composition comprising a compound selected from Table 1, Table 2, Table 3, a pharmaceutically acceptable salt thereof, or a deuterated form thereof.
25. A method of treating a disease comprising subcutaneously administrating a pharmaceutical composition comprising a compound of formula (I), or formula (II), or a pharmaceutically acceptable salt thereof, or a deuterated form thereof,

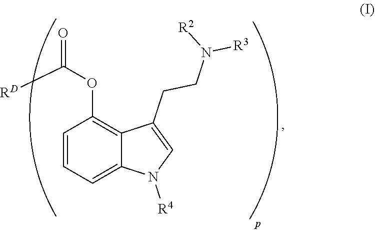

wherein
   R² and R³ are independently alkyl;
   R⁴ is H or C(═O)Oalkyl;
   p is 2 or 3;
   R^{D} is a divalent or trivalent group selected from alkyl, alkenyl, alkylene-O-alkylene, —(CH₂)ₙ—(OCH₂CH₂)ₘ—, cycloalkyl, aryl or

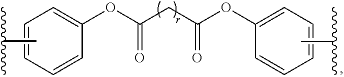

each of which is optionally substituted with OH, Oalkyl, NH₂, NH(alkyl), N(alkyl)₂, COOH, C(═O)Oalkyl, COOH, cycloalkyl or C(═O)alkyl;
n is 1, 2, 3, 4, 5, 6, 7 or 8; m is 1, 2 or 3; and
r is 1, 2, 3;
or

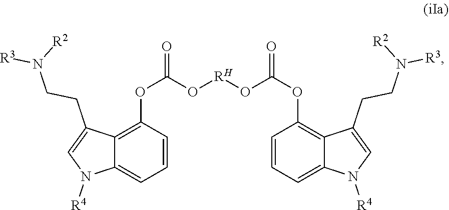

or a pharmaceutically acceptable salt or deuterated form thereof,
wherein:
   each R² and R³ are independently alkyl;
   each R⁴ is independently H or C(═O)Oalkyl;
   R^{H} is —(CH₂)ₙ—(OCH₂CH₂)ₘ— or alkylene;
   n is 1, 2, or 3, and
   m is 1, 2 or 3;
26. The method of any one of embodiments 22-25, wherein the disease is a 5-HT2A receptor associated disease or disorder.
27. The method of embodiment 26, wherein the neuropsychiatric disease is selected from anxiety disorder, attention deficit hyperactivity disorder (ADHD), depression (including treatment resistant depression), cluster headache, diminished drive, burn-out, bore-out, migraine, Parkinson's disease, schizophrenia, an eating disorder (including anorexia nervosa), psychotic disorder, schizophrenia, schizophreniform disorder, schizoaffective disorder, bipolar I disorder, bipolar II disorder, major depressive disorder, psychotic depression, delusional disorders, shared psychotic disorder, Shared paranoia disorder, brief psychotic disorder, paranoid personality disorder, schizoid personality disorder, schizotypal personality disorder, anxiety disorder, social anxiety disorder, substance-induced anxiety disorder, selective mutism, panic disorder, panic attacks, agoraphobia, attention deficit syndrome, posttraumatic stress disorder (PTSD), premenstrual dysphoric disorder (PMDD), and premenstrual syndrome (PMS).

### EXAMPLES

### Example 1. Synthesis of Compound 101: bis(3-(2-(dimethylamino)ethyl)-1H-indol-4-yl) (trans)-cyclohexane-1,4-dicarboxylate, 2Formic Acid

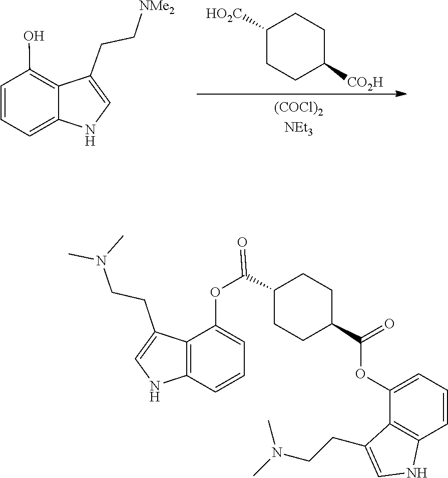 [SMILES: CN(C)CCc1c[nH]c2cccc(O)c12 ; O=C(O)[C@H]1CC[C@H](C(=O)O)CC1 ; CN(C)CCc1c[nH]c2cccc(OC(=O)[C@H]3CC[C@H](C(=O)Oc4cccc5[nH]cc(CCN(C)C)c45)CC3)c12]

To a stirred solution of (trans)-cyclohexane-1,4-dicarboxylic acid (89.9 mg, 1 Eq, 522 μmol) in dry DCM (3 mL) under an atmosphere of nitrogen at r.t. was added oxalyl chloride (278 mg, 192 μL, 4.2 Eq, 2.19 mmol) and a drop of DMF. The reaction mixture was stirred at r.t. for 2 h. The volatiles were removed in vacuo. The residue was dissolved in DCM (2 mL) and added to a solution of psilocin (217.7 mg, 2 Eq, 1.04 mmol) and triethylamine (370 mg, 510 μL, 7 Eq, 3.66 mmol) in DCM (2 mL) at 0° C. The reaction mixture was stirred at r.t. for 1 h. The reaction mixture was diluted with water (5 mL) and transferred into a separating funnel. The aqueous layer was extracted with DCM (3×10 mL). The combined organic layers were washed with brine (20 mL), dried (Na₂SO₄), filtered and concentrated in vacuo. The crude product was purified by chromatography on RP Flash C18 (24 g cartridge, 5-30% (0.1% formic acid in MeCN)/(0.1% formic acid in water)) to afford bis(3-(2-(dimethylamino)ethyl)-1H-indol-4-yl) (1r,4r)-cyclohexane-1,4-dicarboxylate, 2Formic Acid (90.9 mg, 0.11 mmol, 20%) as a yellow solid. The material was dissolved in acetone (5 mL) and a solution of fumaric acid (26 mg, 2 Eq, 0.22 mmol) in acetone (5 mL) was added. The resulting solid was filtered, washed with acetone (2×5 mL) and dried in a vacuum desiccator for 24 h to afford the title compound (58.3 mg, 86 μmol, 16%) as a beige solid.

m/z 546.7 (M+H)⁺ (ES+)

¹H NMR (500 MHZ, DMSO-d₆) δ 11.10 (s, 2H), 8.22 (s, 2H), 7.25 (dd, J=8.1, 0.8 Hz, 2H), 7.17 (d, J=2.3 Hz, 2H), 7.08-7.02 (m, 2H), 6.66 (dd, J=7.6, 0.8 Hz, 2H), 2.87-2.82 (m, 4H), 2.77 (s, 2H), 2.73-2.66 (m, 4H), 2.36 (s, 12H), 2.31-2.26 (m, 4H), 2.08 (s, 1H), 1.68 (td, J=9.1, 3.2 Hz, 4H). 2× exchangeable 0.5H not observed.

### Example 2. Synthesis of Compound 102: rac-bis(3-(2-(dimethylamino)ethyl)-1H-indol-4-yl) (trans)-cyclobutane-1,2-dicarboxylate, 2Formic Acid

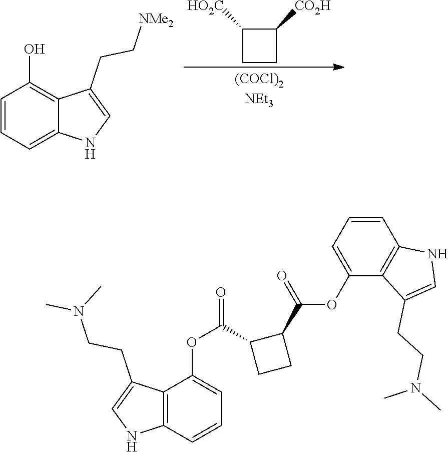

To a stirred solution of trans-cyclobutane-1,2-dicarboxylic acid (14.0 mg, 0.4 Eq, 97.1 μmol) in dry DCM (2 mL) under a nitrogen atmosphere at r.t. was added oxalyl chloride (41 mg, 28 μL, 1.3 Eq, 327 μmol) and a drop of DMF. The reaction mixture was stirred at r.t. for 2 h. The volatiles were removed in vacuo. The residue was redissolved in DCM (1 mL) and added to a solution of psilocin (50.4 mg, 1 Eq, 247 μmol) and triethylamine (247 mg, 340 μL, 10 Eq, 2.44 mmol) in DMF (2 mL) at r.t. The resulting mixture was stirred at r.t. for 2 h. The reaction mixture was diluted with ethyl acetate (10 mL), then water: brine (1:1, 40 mL) and the phases were separated. The organic layer was washed with further water: brine (1:1, 40 mL) and brine (20 mL), dried (Na₂SO₄), filtered and concentrated in vacuo. The crude product was purified by chromatography on RP Flash C18 (12 g cartridge, 5-50% (0.1% formic acid in MeCN)/(0.1% formic acid in water)) to afford the title compound (13.8 mg, 19 μmol, 20%) as a brown gum.

m/z 517.2 (M+H)⁺ (ES+)

¹H NMR (500 MHZ, DMSO-d₆) δ 11.05 (s, 2H), 8.19 (s, 2H), 7.24 (d, J=8.1 Hz, 2H), 7.15 (d, J=2.3 Hz, 2H), 7.04 (dd, J=7.9, 7.9 Hz, 2H), 6.70 (d, J=7.6 Hz, 2H), 3.99-3.90 (m, 2H), 2.79 (dd, J=9.4, 6.3 Hz, 4H), 2.55-2.51 (m, 3H), 2.47-2.42 (m, 2H), 2.40-2.34 (m, 2H), 2.20 (s, 12H). 2× exchangeable H not observed.

### Example 3. Synthesis of Salt of Compound 103: bis(3-(2-(dimethylamino)ethyl)-1H-indol-4-yl) 2,6-dimethylterephthalate, 0.75Formic Acid, 1.25HCl

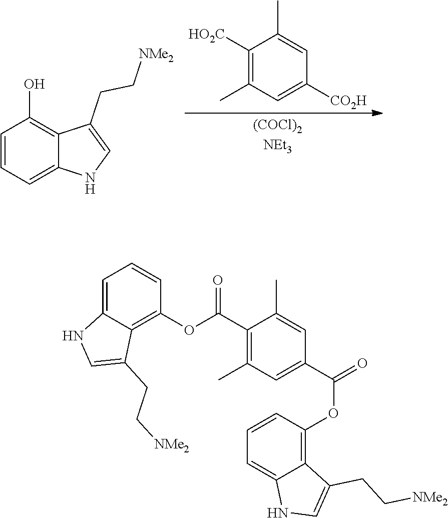 [SMILES: CN(C)CCc1c[nH]c2cccc(O)c12 ; Cc1cc(C(=O)O)cc(C)c1C(=O)O ; Cc1cc(C(=O)Oc2cccc3[nH]cc(CCN(C)C)c23)cc(C)c1C(=O)Oc1cccc2[nH]cc(CCN(C)C)c12]

Oxalyl dichloride (145 mg, 100 μL, 2.7 Eq, 1.14 mmol) was added to a stirred suspension of 2,6-dimethylterephthalic acid (86 mg, 1 Eq, 422 μmol) and one drop of DMF in DCM (4 mL) and left to stir under nitrogen for 3 h. Volatile components were then removed in vacuo and the residue redissolved in DCM (4 mL). Psilocin (200 mg, 2.2 Eq, 929 μmol) and triethylamine (513 mg, 706 μL, 12 Eq, 5.07 mmol) were added and the resultant suspension stirred at r.t. overnight. The bulk solvent was removed in vacuo. The crude material was dissolved in DMSO (1.9 mL), filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters qDa) on a Waters X-Select CSH C18 ODB prep column, 130 Å, 5 μm, 30 mm×100 mm, flow rate 40 mLmin-1 eluting with a 0.1% formic acid in water-MeCN gradient over 12.5 min using UV across all wavelengths with PDA as well as a QDA and ELS detector. At column dilution pump gives 2 mLmin-1 MeCN over the entire method, which is included in the following MeCN percentages. Gradient information: 0.0-0.5 min, 5% MeCN; 0.5-10.5 min, ramped from 5% MeCN to 32.5% MeCN; 10.5-10.6 min, ramped from 32.5% MeCN to 100% MeCN; 10.6-12.5 min, held at 100% MeCN. The clean fractions were evaporated in a Genevac to afford the title compound (23.0 mg, 35 μmol, 6%) as an off-white solid.

m/z 284.0 (M+H)²⁺ (ES+)

¹H NMR (500 MHZ, DMSO-d6) δ 11.13 (d, J=9.0 Hz, 2H), 8.15 (s, 0.75H), 8.05 (s, 2H), 7.31 (dd, J=8.2, 2.4 Hz, 2H), 7.20 (dd, J=9.1, 2.3 Hz, 2H), 7.16-7.10 (m, 2H), 7.05 (d, J=7.6 Hz, 1H), 6.83 (d, J=7.6 Hz, 1H), 2.81-2.73 (m, 4H), 2.59 (s, 6H), 2.46 (d, J=7.4 Hz, 2H), 2.07 (s, 6H), 2.00 (s, 6H). 4×H obscured by solvent peak. 2× exchangeable H not observed.

### Example 4. Synthesis of Compound 104: bis(3-(2-(dimethylamino)ethyl)-1H-indol-4-yl) 2-methoxyisophthalate, 2Formic Acid

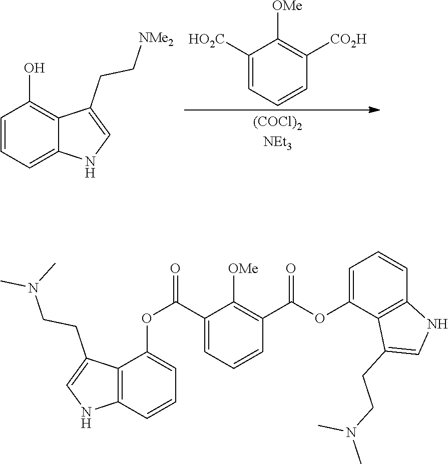 [SMILES: CN(C)CCc1c[nH]c2cccc(O)c12 ; COc1c(C(=O)O)cccc1C(=O)O ; COc1c(C(=O)Oc2cccc3[nH]cc(CCN(C)C)c23)cccc1C(=O)Oc1cccc2[nH]cc(CCN(C)C)c12]

To a stirred solution of 2-methoxyisophthalic acid (21 mg, 1 Eq, 109 μmol) in dry DCM (4 mL) under a nitrogen atmosphere at r.t. was added oxalyl chloride (58 mg, 40 μL, 4.2 Eq, 456 μmol) and a drop of DMF. The reaction mixture was stirred at r.t. for 2 h. Volatiles were removed in vacuo. The residue was redissolved in DCM (2 mL) and added to a solution of psilocin (50.4 mg, 2 Eq, 217 μmol) and triethylamine (77 mg, 106 μL, 7 Eq, 760 μmol) in DCM (2 mL) at 0° C. The reaction mixture was stirred at r.t. for 16 h. The reaction mixture was diluted with water (5 mL) and transferred into a separating funnel. The aqueous layer was extracted with DCM (3×10 mL). The combined organic layers were washed with brine (20 mL), dried (Na₂SO₄), filtered and concentrated in vacuo. The crude product was purified by chromatography on RP Flash C18 (24 g cartridge, 5-20% (0.1% formic acid in MeCN)/(0.1% formic acid in Water)) to afford the title compound (3.0 mg, 3.6 μmol, 3%) as a dark brown gum.

m/z 567.9 (M+H)⁺ (ES+)

¹H NMR (500 MHZ, DMSO-d₆) δ 11.14 (s, 2H), 8.38 (d, J=7.8 Hz, 2H), 8.19 (s, 2H), 7.59-7.53 (m, 1H), 7.31 (dd, J=8.2, 0.8 Hz, 2H), 7.20 (d, J=2.3 Hz, 2H), 7.16-7.09 (m, 2H), 6.87 (dd, J=7.6, 0.8 Hz, 2H), 3.99 (s, 3H), 2.84-2.77 (m, 4H), 2.57-2.52 (m, 4H), 2.09 (s, 12H). 2× exchangeable H not observed.

### Example 5. Synthesis of Compound 105: bis(3-(2-(dimethylamino)ethyl)-1H-indol-4-yl) 3,3′-oxydipropionate, 2Formic Acid

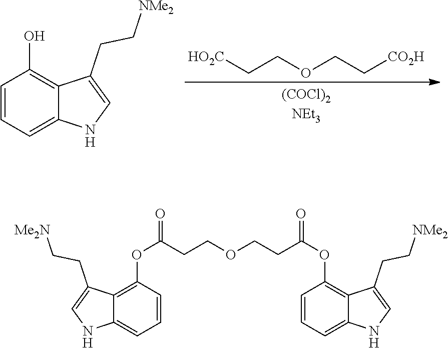 [SMILES: CN(C)CCc1c[nH]c2cccc(O)c12 ; O=C(O)CCOCCC(=O)O ; CN(C)CCc1c[nH]c2cccc(OC(=O)CCOCCC(=O)Oc3cccc4[nH]cc(CCN(C)C)c34)c12]

To a stirred solution of 3,3′-oxydipropionic acid (81 mg, 1 Eq, 502 μmol) in dry DCM (4 mL) under an atmosphere of nitrogen at r.t. was added oxalyl dichloride (268 mg, 185 μL, 4.2 Eq, 2.11 mmol) and a drop of DMF. The reaction mixture was stirred at r.t. for 2 h. The volatiles were removed in vacuo. The residue was dissolved in DCM (2 mL) and added to a solution of psilocin (205.2 mg, 2 Eq, 1.01 mmol) and triethylamine (356 mg, 490 μL, 7 Eq, 3.52 mmol) in DCM (2 mL) at 0° C. The reaction mixture was stirred at r.t. for 17 h. The reaction mixture was diluted with water (5 mL) and transferred into a separating funnel. The aqueous layer was extracted with DCM (3×10 mL). The combined organic layers were washed with brine (20 mL), dried (Na₂SO₄), filtered and concentrated in vacuo. The crude product was purified by chromatography on RP Flash C18 (24 g cartridge, 5-30% (0.1% formic acid in MeCN)/(0.1% formic acid in water)) to afford bis(3-(2-(dimethylamino)ethyl)-1H-indol-4-yl) 3,3′-oxydipropionate, 2Formic Acid (55.7 mg, 80 μmol, 16%, 90% Purity) as a red oil. The material was dissolved in acetone (3 mL) and a solution of fumaric acid (19 mg, 0.3 Eq, 160 μmol) in acetone (3 mL) was added. Volatiles were removed in vacuo and the material was purified by chromatography on RP Flash C18 (12 g cartridge, 5-25% (0.1% formic acid in MeCN)/(0.1% formic acid in water)) to afford the title compound (12.2 mg, 17 μmol, 3%) as a dark green oil.

m/z 534.9 (M+H)⁺ (ES+)

¹H NMR (500 MHZ, DMSO-d₆) δ 11.04 (s, 2H), 8.20 (s, 2H), 7.23 (d, J=8.2 Hz, 2H), 7.15 (d, J=2.4 Hz, 2H), 7.01-6.97 (m, 2H), 6.64 (d, J=7.4 Hz, 2H), 3.85 (t, J=6.2 Hz, 4H), 2.95 (t, J=6.2 Hz, 4H), 2.82-2.77 (m, 4H), 2.56-2.53 (m, 4H), 2.26 (s, 12H). 2× exchangeable H not observed.

### Example 6. Synthesis of Compound 106: bis(3-(2-(dimethylamino)ethyl)-1H-indol-4-yl) 3-methylpentanedioate, 2Fumaric acid, acetone

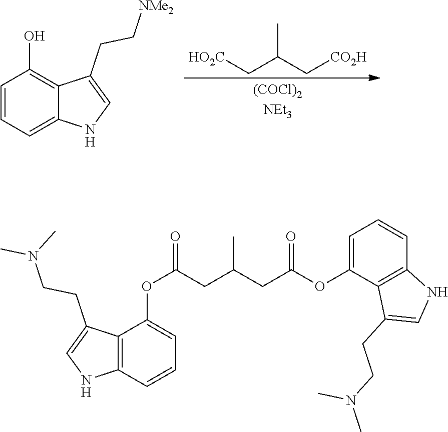

To a stirred solution of 3-methylpentanedioic acid (67 mg, 1 Eq, 459 μmol) in dry DCM (4 mL) under a nitrogen atmosphere at r.t. was added oxalyl chloride (245 mg, 169 μL, 4.2 Eq, 1.93 mmol) and a drop of DMF. The reaction mixture was stirred at r.t. for 2 h. Volatiles were removed in vacuo. The residue was redissolved in DCM (2 mL) and added to a solution of psilocin (213 mg, 2 Eq, 918 μmol) and triethylamine (325 mg, 448 μL, 7 Eq, 3.21 mmol) in DCM (2 mL) at 0° C. The reaction mixture was stirred at r.t. for 16 h. The reaction mixture was diluted with water (5 mL) and transferred into a separating funnel. The aqueous layer was extracted with DCM (3×10 mL). The combined organic layers were washed with brine (20 mL), dried (Na₂SO₄), filtered and concentrated in vacuo. The crude product was purified by chromatography on RP Flash C18 (24 g cartridge, 5-20% (0.1% formic acid in MeCN)/(0.1% formic acid in water)) and dried in a vacuum desiccator at 50° C. overnight to afford bis(3-(2-(dimethylamino)ethyl)-1H-indol-4-yl) 3-methylpentanedioate, 2Formic Acid (65.0 mg, 87 μmol, 19%) as a sticky brown gum. The partially purified material was dissolved in acetone (2 mL) was added a solution of fumaric acid (10 mg, 1 Eq, 89.5 μmol) in acetone (2 mL). The mixture was left in the freezer for 2 days, then left to stand at r.t. for 2 days. The liquid was decanted and the solid was removed from the vial, and dried under vacuum to afford the title compound (14.0 mg, 16 μmol, 3%) as a light brown solid.

m/z 519.3 (M+H)⁺ (ES+)

¹H NMR (500 MHz, DMSO-d₆) δ 11.12 (d, J=1.7 Hz, 2H), 7.25 (dd, J=8.1, 0.8 Hz, 2H), 7.19 (d, J=2.4 Hz, 2H), 7.08-7.01 (m, 2H), 6.69 (dd, J=7.7, 0.8 Hz, 2H), 6.55 (s, 4H), 2.94-2.86 (m, 6H), 2.82-2.70 (m, 6H), 2.62-2.54 (m, 1H), 2.42 (s, 12H), 2.08 (s, 6H), 1.19 (d, J=6.6 Hz, 3H). 4× exchangeable H not observed.

### Example 7. Synthesis of Compound 118: tris(3-(2-(dimethylamino)ethyl)-1H-indol-4-yl) cyclohexane-1,3,5-tricarboxylate, 1.5Formic Acid, 1.5Fumaric acid and tris(3-(2-(dimethylamino)ethyl)-1H-indol-4-yl) cyclohexane-1,3,5-tricarboxylate, 3Formic Acid

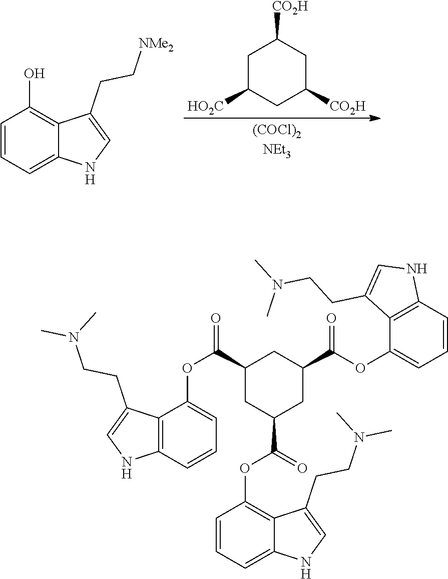 [SMILES: CN(C)CCc1c[nH]c2cccc(O)c12 ; O=C(O)[C@H]1C[C@@H](C(=O)O)C[C@@H](C(=O)O)C1 ; CN(C)CCc1c[nH]c2cccc(OC(=O)[C@H]3C[C@@H](C(=O)Oc4cccc5[nH]cc(CCN(C)C)c45)C[C@@H](C(=O)Oc4cccc5[nH]cc(CCN(C)C)c45)C3)c12]

To a stirred solution of cyclohexane-1,3,5-tricarboxylic acid (70 mg, 1 Eq, 324 μmol) in dry DCM (3 mL) under an atmosphere of nitrogen at r.t. was added oxalyl chloride (181 mg, 125 μL, 4.4 Eq, 1.42 mmol) and a drop of DMF. The reaction mixture was stirred at r.t. for 2 h. The volatiles were removed in vacuo. The residue was dissolved in DCM (2 mL) and added to a solution of psilocin (202.0 mg, 3 Eq, 969 μmol) and triethylamine (295 mg, 406 μL, 9 Eq, 2.91 mmol) in DCM (2 mL) at 0° C. The reaction mixture was stirred at r.t. for 1 h. The reaction mixture was diluted with water (5 mL) and transferred into a separating funnel. The aqueous layer was extracted with DCM (3×10 mL). The combined organic layers were washed with brine (20 mL), dried (Na₂SO₄), filtered and concentrated in vacuo. The crude product was purified by chromatography on RP Flash C18 (24 g cartridge, 5-50% (0.1% formic acid in MeCN)/(0.1% formic acid in water)) to afford tris(3-(2-(dimethylamino)ethyl)-1H-indol-4-yl) cyclohexane-1,3,5-tricarboxylate, 3Formic Acid (91.2 mg, 95 μmol, 29%) as a beige oil.

m/z 775.5 (M+H)⁺ (ES+)

¹H NMR (500 MHZ, DMSO-d₆) δ 11.06 (s, 3H), 8.19 (s, 3H), 7.25 (dd, J=8.0, 4.7 Hz, 3H), 7.16 (d, J=2.4 Hz, 3H), 7.09-7.01 (m, 3H), 6.69 (dd, J=11.6, 7.6 Hz, 3H), 3.17-3.09 (m, 3H), 2.81 (q, J=9.7 Hz, 6H), 2.71 (d, J=12.5 Hz, 2H), 2.54 (d, J=5.1 Hz, 7H), 2.23 (d, J=9.3 Hz, 18H), 1.88 (q, J=12.7 Hz, 3H). 3× exchangeable H not observed.

The material was dissolved in acetone (3 mL) and a solution of fumaric acid (38 mg, 1.0 Eq, 0.33 mmol) in acetone (4 mL) was added. The resulting solid was filtered, washed with acetone (2 mL) and dried in a vacuum desiccator for 6 h to afford tris(3-(2-(dimethylamino)ethyl)-1H-indol-4-yl) cyclohexane-1,3,5-tricarboxylate, 1.5Formic Acid, 1.5Fumaric acid (13.4 mg, 13 μmol, 4%) as a beige solid.

m/z 775.5 (M+H)⁺ (ES+)

¹H NMR (500 MHZ, DMSO-d₆) δ 11.11 (s, 3H), 8.20 (s, 2H), 7.28-7.22 (m, 3H), 7.18 (d, J=2.5 Hz, 3H), 7.09-7.02 (m, 3H), 6.72-6.67 (m, 3H), 6.54 (s, 3H), 3.18 (t, J=12.9 Hz, 3H), 2.94-2.82 (m, 7H), 2.72 (dd, J=20.3, 11.5 Hz, 8H), 2.35 (d, J=3.1 Hz, 18H), 2.08 (s, 5H, 1 acetone solvate), 1.86 (q, J=12.7 Hz, 3H). 6× exchangeable 0.5H not observed.

### Example 8. Synthesis of compound 119: bis(3-(2-(dimethylamino)ethyl)-1H-indol-4-yl) ((ethane-1,2-diylbis(oxy))bis(ethane-2,1-diyl)) bis(carbonate), 2Fumaric acid

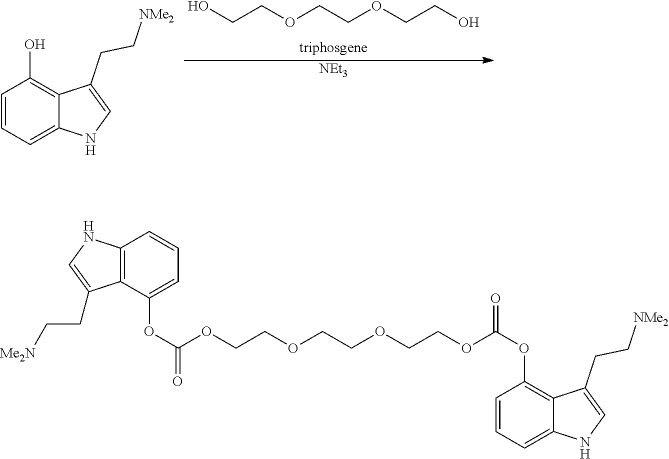

To a stirred solution of triphosgene (198 mg, 1 Eq, 666 μmol) and pyridine (293 mg, 300 μL, 5.6 Eq, 3.71 mmol) in dry DCM (2 mL) at 0° C. was added 2,2′-(ethane-1,2-diylbis(oxy))bis(ethan-1-ol) (100 mg, 1 Eq, 666 μmol) in dry DCM (1 mL) and the reaction mixture was stirred for 2 h at 0° C. Psilocin (272 mg, 2 Eq, 1.33 mmol) in DMF (2 mL) was added and the reaction mixture was stirred for 2 h at 0° C. then for 16 h at r.t. The mixture was diluted with DCM (5 mL) and water (2 mL) and passed through a phase-sep cartridge. The crude product was purified by chromatography on RP Flash C18 (24 g cartridge, 0-40% (0.1% formic acid in MeCN)/(0.1% formic acid in water)) (eluting 15%) to afford partially purified bis(3-(2-(dimethylamino)ethyl)-1H-indol-4-yl) ((ethane-1,2-diylbis(oxy))bis(ethane-2,1-diyl)) bis(carbonate), 2formic acid (24.0 mg, 20.0 μmol, 3%) as a thick brown oil. The material was dissolved in acetone (2 mL) and a solution of fumaric acid (9 mg, 3.8 Eq, 77.5 μmol) in acetone (2 mL) was added. The mixture was cooled at −20° C. for 48 h. The resulting solid was isolated by filtration, washed with MeCN (2×2 mL), and dried in vacuo to afford the title compound (10.0 mg, 12 μmol, 2%) as a grey solid.

m/z 611.3 (M+H)⁺ (ES+)

¹H NMR (500 MHZ, DMSO-d₆) δ 11.13 (s, 2H), 7.27 (d, J=8.1 Hz, 2H), 7.20 (d, J=2.3 Hz, 2H), 7.05 (dd, J=7.9, 7.9 Hz, 2H), 6.78 (d, J=7.6 Hz, 2H), 6.55 (s, 4H), 4.40-4.30 (m, 4H), 3.75-3.68 (m, 4H), 3.61 (s, 4H), 2.87-2.79 (m, 4H), 2.70-2.59 (m, 4H), 2.35 (s, 12H). 4× exchangeable H not observed.

### Example 9. Synthesis of Compound 120: bis(3-(2-(dimethylamino)ethyl)-1H-indol-4-yl) octane-1,8-diyl bis(carbonate), 1.5Fumaric acid, 0.5HCl

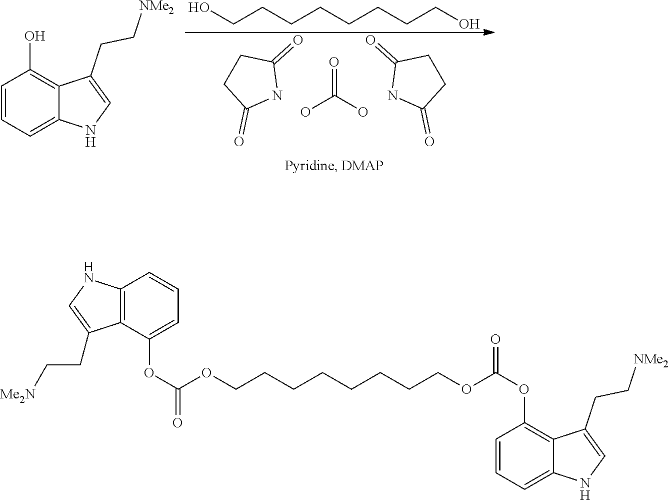

To a solution of psilocin (205.8 mg, 1 Eq, 1.01 mmol) and N.N′-disuccinimidyl carbonate (336 mg, 1.3 Eq, 1.31 mmol) in dry DCM (7 mL) at r.t. under a nitrogen atmosphere, was added pyridine (319 mg, 0.33 mL, 4 Eq, 4.03 mmol). The reaction mixture was stirred for 1 h at r.t. To the reaction mixture, were added octane-1,8-diol (74 mg, 0.5 Eq, 504 μmol) and N,N-dimethylpyridin-4-amine (123 mg, 1 Eq, 1.01 mmol) and the mixture was stirred at r.t. for 1 h. The mixture was then heated at 38° C. overnight. The reaction mixture was diluted with DCM (10 mL) and water (15 mL) and transferred into a separating funnel. The aqueous layer was extracted with DCM (3×15 mL). The combined organic layers were washed with brine (30 mL), dried (Na₂SO₄), filtered and concentrated in vacuo. The crude product was purified by chromatography on RP Flash C18 (24 g cartridge, 5-40% (0.1% formic acid in MeCN)/(0.1% formic acid in water)) to afford partially purified bis(3-(2-(dimethylamino)ethyl)-1H-indol-4-yl) octane-1,8-diyl bis(carbonate), formic acid (45 mg, 51 μmol, 5%) as a sticky brown oil. The material was dissolved in acetone (2 mL) and a solution of fumaric acid (16 mg, 0.14 Eq, 139 μmol) in acetone (3 mL) was added. The resulting solid was filtered, washed with acetone (2 mL) and dried in a vacuum desiccator for 24 h to afford the title compound (18.9 mg, 23 μmol, 2%) as a grey solid.

m/z 607.4 (M+H)⁺ (ES+)

¹H NMR (500 MHZ, DMSO) δ 11.16 (s, 2H), 7.27 (dd, J=8.1, 0.8 Hz, 2H), 7.20 (d, J=2.3 Hz, 2H), 7.09-7.02 (m, 2H), 6.78 (dd, J=7.7, 0.8 Hz, 2H), 6.55 (s, 3H), 4.23 (t, J=6.6 Hz, 4H), 2.87 (dd, J=9.8, 6.1 Hz, 4H), 2.75 (dd, J=9.6, 6.3 Hz, 4H), 2.40 (s, 12H), 1.68 (p, J=6.7 Hz, 4H), 1.42-1.23 (m, 8H). 3× exchangeable H not observed.

### Example 10. Synthesis of Compound 122: bis(3-(2-(dimethylamino)ethyl)-1H-indol-4-yl) glutarate, 1.75Fumaric acid, 0.25HCl

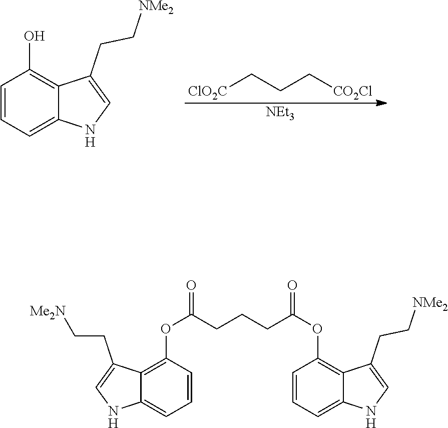

To a suspension of psilocin (200 mg, 1 Eq, 979 μmol) in DMF (4 mL) was added triethylamine (0.59 g, 0.8 mL, 6 Eq, 5.9 mmol). glutaroyl dichloride (86 mg, 65 μL, 0.52 Eq, 509 μmol) was added dropwise and the reaction was stirred at r.t. for 18 h. The mixture was poured into ice/water (40 mL) and extracted with EtOAc (2×20 mL). The combined organics were washed sequentially with water: brine (50 mL), brine (50 mL), then dried (Na₂SO₄) and concentrated in vacuo. The crude product was purified by chromatography on RP Flash C18 (40 g cartridge, 5-40% (0.1% formic acid in MeCN)/(0.1% formic acid in water)) (eluting ˜10%) to afford bis(3-(2-(dimethylamino)ethyl)-1H-indol-4-yl) glutarate, 2Formic Acid (125 mg, 227 μmol, 46%) as a pale brown oil. To a solution of the product (123 mg, 1 Eq, 206 μmol) in acetone (15 mL) was added a solution of fumaric acid (48 mg, 2 Eq, 414 μmol) in acetone (3 mL). Initially a solid precipitated from the acetone, but this became a gum on isolation. The material was sonicated in MeCN (10 mL). The resulting solid was isolated by filtration and dried in vacuo to afford the title compound (82.1 mg, 115 μmol, 56%) as a white solid. A further solid precipitated to afford bis(3-(2-(dimethylamino)ethyl)-1H-indol-4-yl) glutarate, 2Fumaric acid (15.8 mg, 21 μmol, 12%) as a white solid.

m/z 505.3 (M+H)⁺ (ES+)

¹H NMR (500 MHZ, DMSO-d₆) δ 11.09 (s, 2H), 7.24 (d, J=8.1 Hz, 2H), 7.18 (d, J=2.3 Hz, 2H), 7.05 (t, J=7.9 Hz, 2H), 6.71 (d, J=7.6 Hz, 2H), 6.55 (s, 3.5H), 2.91-2.82 (m, 8H), 2.71 (t, J=7.9 Hz, 4H), 2.37 (s, 12H), 2.09 (p, J=7.5 Hz, 2H). 4× exchangeable H not observed.

¹H NMR (500 MHZ, DMSO-d₆) δ 11.09 (s, 2H), 7.24 (d, J=8.1 Hz, 2H), 7.18 (d, J=2.3 Hz, 2H), 7.05 (dd, J=7.9, 7.9 Hz, 2H), 6.71 (d, J=7.6 Hz, 2H), 6.56 (s, 4H), 2.88-2.81 (m, 8H), 2.69 (t, J=7.9 Hz, 4H), 2.36 (s, 12H), 2.12-2.05 (m, 2H). 4× exchangeable H not observed.

### Example 11. Synthesis of Compound 123: bis(3-(2-(dimethylamino)ethyl)-1H-indol-4-yl) adipate, 1.75Fumaric acid, 0.25Formic Acid

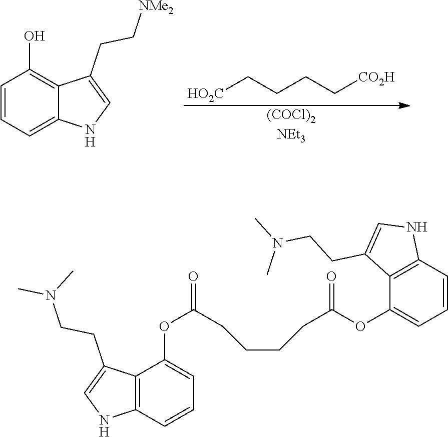 [SMILES: CN(C)CCc1c[nH]c2cccc(O)c12 ; O=C(O)CCCCC(=O)O ; CN(C)CCc1c[nH]c2cccc(OC(=O)CCCCC(=O)Oc3cccc4[nH]cc(CCN(C)C)c34)c12 ; "(COCl)2NEt3"]

To a stirred solution of adipic acid (71.8 mg, 1 Eq, 492 μmol) in dry DCM (4 mL) under an atmosphere of nitrogen at r.t. was added oxalyl chloride (262 mg, 181 μL, 4.2 Eq, 2.06 mmol) and a drop of DMF. The reaction mixture was stirred at r.t. for 2 h. Volatiles were removed in vacuo. The residue was dissolved in DCM (2 mL) and added to a solution of psilocin (200.8 mg, 2 Eq, 983 μmol) and triethylamine (348 mg, 480 μL, 7 Eq, 3.44 mmol) in DCM (2 mL) at 0° C. The reaction mixture was stirred at r.t. for 17 h. The reaction mixture was diluted with water (5 mL) and transferred into a separating funnel. The aqueous layer was extracted with DCM (3×10 mL). The combined organic layers were washed with brine (20 mL), dried (Na₂SO₄), filtered and concentrated in vacuo. The crude product was purified by chromatography on RP Flash C18 (24 g cartridge, 5-30% (0.1% formic acid in MeCN)/(0.1% formic acid in Water)) to afford bis(3-(2-(dimethylamino)ethyl)-1H-indol-4-yl) adipate, 2Formic Acid (58.6 mg, 91 μmol, 19%) as a red oil. The material was dissolved in acetone (3 mL) and a solution of fumaric acid (22 mg, 2 Eq, 192 μmol) in acetone (4 mL) was added. The resulting solid was filtered, washed with acetone (2×5 mL) and dried in a vacuum desiccator for 24 h to afford the title compound (42.8 mg, 57 μmol, 12%) as a red solid.

m/z 519.3 (M+H)⁺ (ES+)

¹H NMR (500 MHZ, DMSO-d₆) δ 11.09 (s, 2H), 8.18 (s, 0.25H), 7.24 (d, J=8.1 Hz, 2H), 7.17 (d, J=2.4 Hz, 2H), 7.07-7.01 (m, 2H), 6.68 (d, J=7.6 Hz, 2H), 6.54 (s, 3.5H), 2.91-2.84 (m, 4H), 2.83-2.71 (m, 8H), 2.41 (s, 12H), 1.80 (s, 4H). 2× exchangeable 0.5H not observed.

### Example 12. Synthesis of Compounds 124: bis(3-(2-(dimethylamino)ethyl)-1H-indol-4-yl) heptanedioate, 2Formic Acid

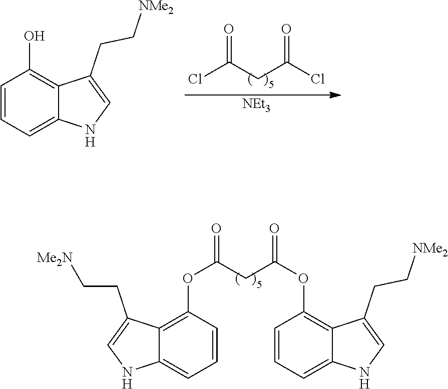

To a solution of psilocin (200 mg, 1 Eq, 979 μmol) and triethylamine (119 mg, 1.2 Eq, 1.17 mmol) in DMF (5 mL) at 0° C. was added dropwise heptanedioyl dichloride (96.5 mg, 0.5 Eq, 490 μmol). The reaction mixture was stirred at r.t. for 16 h. The mixture was diluted with ethyl acetate (20 mL). Water/brine (1:1, 50 mL) was added and the phases were separated. The aqueous phase was extracted with EtOAc (20 mL). The combined organics were washed with water/brine (1:1, 50 mL), brine (50 mL), dried (Na₂SO₄) and concentrated in vacuo. The crude product was loaded onto celite and purified by chromatography on RP Flash C18 (24 g cartridge, 5-30% (0.1% formic acid in MeCN)/(0.1% formic Acid in water)) to afford the title compound (30.0 mg, 46 μmol, 5%) as a sticky yellow oil.

m/z 533.3 (M+H)⁺ (ES+)

¹H NMR (500 MHZ, DMSO) δ 11.05 (m, 2H), 8.19 (s, 2H), 7.23 (d, J=8.1 Hz, 2H), 7.16 (d, J=2.3 Hz, 2H), 7.06-7.00 (m, 2H), 6.66 (d, J=7.6 Hz, 2H), 2.83-2.76 (m, 4H), 2.72 (t, J=7.4 Hz, 4H), 2.61-2.55 (m, 4H), 2.28 (s, 12H), 1.76 (app. p, J=7.5 Hz, 4H), 1.57-1.48 (m, 2H). 2× exchangeable H not observed.

### Example 13. Synthesis of Compound 167:3-(3-((3-(2-(dimethylamino)ethyl)-1H-indol-4-yl)oxy)-3-oxopropoxy) propanoic acid

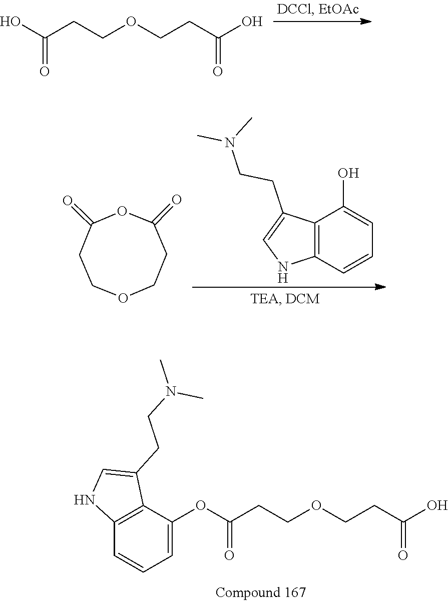 [SMILES: O=C(O)CCOCCC(=O)O ; O=C1CCOCCC(=O)O1 ; CN(C)CCc1c[nH]c2cccc(O)c12 ; CN(C)CCc1c[nH]c2cccc(OC(=O)CCOCCC(=O)O)c12]

### Step 1: Preparation of 1,5-dioxocane-2,8-dione

3,3′-Oxydipropionic acid (5.00 g, 30.8 mmol, 1.00 eq) was added to a stirred ice-cold solution of N,N′-dicyclohexylcarbodiimide (7.00 g, 33.9 mmol, 1.10 eq) in EtOAc (50 mL) and the resulting mixture stirred under nitrogen for 18 h, warming to room temperature. The resulting white suspension was filtered through a pad of Celite® to remove solid by-products and the filter cake rinsed with EtOAc (20 mL). The filtrates were evaporated in vacuo to give the crude title compound (4.949 g, 97%) as a colourless oil. 1H NMR analysis showed that the product was a mixture of the expected cyclic anhydride plus other, possibly linear anhydride, impurities The material was used without purification.

### Step 2: Preparation of Compound 167:3-(3-((3-(2-(dimethylamino)ethyl)-1H-indol-4-yl)oxy)-3-oxopropoxy) propanoic acid

A stirred solution of 1,5-dioxocane-2,8-dione (8, 4.51 g, 31.3 mmol, 2.13 eq), and TEA (6.3 mL, 45.2 mmol, 3.07 eq) in dry DCM (30 mL) was added at room temperature to a stirred suspension of psilocin (3.004 g, 14.7 mmol, 1.00 eq) in dry dichloromethane (30 mL) and the resulting dark red solution stirred for 4.5 h. The reaction mixture was concentrated in vacuo to give a dark red gum that was dissolved in DMSO and the solution purified by preparative reverse-phase chromatography using a Puriflash® PF-15C18HP-0330 column and a gradient acidic mobile phase of 3-40% MeCN in water containing 0.1 v/v % formic acid, with UV detection at 218 nm. Pure product fractions were combined and freeze dried to give the product as colourless glass that was triturated with MeCN (5 mL) to give 3-(3-((3-(2-(dimethylamino)ethyl)-1H-indol-4-yl)oxy)-3-oxopropoxy) propanoic acid (Compound 167, 888 mg, 17%).

¹H NMR (400 MHZ, DMSO-d6) δ (ppm): 11.04 (1H, bs), 7.23 (1H, d, J=7.8 Hz), 7.14 (1H, d, J=2 Hz), 7.03 (1H, t, J=7.9 Hz), 6.65 (1H, d, J=7.3 Hz), 3.76 (2H, t, J=6 Hz), 3.65 (2H, t, J=6 Hz), 2.90 (2H, t, J=6 Hz), 2.85-2.79 (2H, m), 2.65-2.58 (2H, m), 2.38 (2H, t, J=6 Hz), 2.31 (6H, s), acid proton not observed due to exchange with water in DMSO-d6 solvent. UPLC-MS (Method in Appendix 1): Rt=3.50 min, [MH]+=349.3

### Example 14. Psilocin Release Studies

Plasma stability and psilocin release of compounds were assessed by monitoring the disappearance of parent compounds for up to 2 hours at 37° C. in plasma (human or rat) in duplicate, using positive (Propantheline), negative (Pepstatin) and solvent controls (DMSO) to confirm suitability of the assay. Psilocin was also run as a control to monitor psilocin formation in a semi-quantitative manner.

Samples were analysed by UHPLC-MS/MS-Sciex™ MS500 Triple Quad QTRAP UHPLC system with a HESI-II electrospray source on a Waters™ Acquity UPLC® HSS T3 column (1.8 μm, 2.1 mm×50 mm), mobile were phases water+0.1% formic acid and methanol+0.1% formic acid.

The elimination rate constant and half-life (t_{1/2}) were determined using Ln (MS response) vs time plots. In addition, the appearance of psilocin from test compounds was monitored and assessed (as a percentage) against control psilocin peaks (at Time 0) to provide a semi-quantitative measure of psilocin release. The psilocin release data are included in table 5.

TABLE 5
Psilocin Release

| Compound | Maximum Psilocin | |
|---|---|---|
| No. | Release (%) | Psilocin Release Efficiency (%) ^{[1]} |
| 101 | 76 | 38 |
| 102 | 125 | 62.5 |
| 103 | 14 | 7 |
| 104 | 27 | 13.5 |
| 105 | 172 | 81 |
| 106 | 80 | 40 |
| 119 | 200 | 100 |
| 120 | 200 | 100 |
| 121 | 100 | 50 |
| 118 | 30 | 10 |
| 167 ^{[2]} | 100 | 100 |

^{[1]} Psilocin Release Efficiency (%) is calculated by the following equation:
$Psilocin release efficiency ( \% ) = \frac{\begin{matrix}actual human plasma \\ maximum psilocin release\end{matrix}}{\begin{matrix}theoretical maximum human \\ plasma psilocin release\end{matrix}} \times 100 \%$ ^{[2]} psilocin release data for this compound was obtained from rat plasma experiments.

### Example 15. Psilocin Pharmacokinetics of Compound 122

The psilocin pharmacokinetics of compound 122 were studied in the plasma of male Sprague-Dawley (SD) rats (n=3), 0.083-4 hours, following a subcutaneous (SC) dose that delivered 0.72 mg/kg psilocin (assuming 100% conversion of the pro-drug). Additionally, brain samples (n=3) were taken at approximately 1 hour to determine psilocin concentration achieved in the brain. Psilocin was quantified by high performance liquid chromatography with tandem mass spectrometry (HPLC-MS/MS) against matrix matched calibration curves.

Animals: Male Sprague-Dawley rats (300-325 g at time of arrival) were housed in groups of 2 or 3, and on a normal 12-hour light-dark cycle. Relative humidity (RH) was typically 55±15% with prolonged periods below 40% RH or above 70% RH avoided. Animals had free access to standard maintenance diet and tap water ad libitum throughout the study.

Compounds: Compound 122 was dissolved in saline (1.3 mg/kg) and adjusted to pH 7.4 for SC administration to the neck scruff at a dosing volume of 5 mL/kg. The final pH was checked prior to dosing, and found to be pH 6.3. This dose was selected as it delivered 0.72 mg/kg psilocin (assuming 100% conversion of the pro-drug). Aliquots of the formulation was additionally collected and stored at −80° C. until analysis by high performance liquid chromatography with tandem mass spectrometry (HPLC-MS/MS) to confirm dosing of compound 122 and minimal conversion to psilocin pre-dose (<2.5%).

Sample collection and bioanalysis: At 0.083, 0.11, 0.25, 0.5, 0.75, 1, 2, 4 hours post dose, blood samples were collected from the lateral tail vein in K3EDTA-coated tubes and spun to collect plasma within 30 minutes of collection (samples were held on ice prior to centrifugation). Plasma samples were aliquoted and supplemented with DTT (10:1, plasma: DTT) to give a final concentration in the plasma samples of 5 mM DTT. Plasma samples were stored on dry ice, then transferred to −80° C., prior to analysis.

For the 1-hour brain samples, following confirmation of death the brain was collected, rinsed in PBS, blotted dry and then snap frozen. Brain samples were initially stored on dry ice, then transferred to −80° C., prior to analysis.

To quantify any psilocin present in the dose formulations, psilocin and compound 122 standards were prepared in water. Three aliquots from the dose formulations were diluted to a nominal concentration of 5 μg/mL, and injected in triplicate into the HPLC-MS/MS. The relative percentage of the psilocin peak in the dose formulation compared to the psilocin standard peak at 5 μg/mL, was used to assess for psilocin conversion in the dosing formulations.

Plasma and brain samples were extracted using acetonitrile protein crash containing internal standard (100 ng/mL psilocin d₁₀). Brain samples were homogenized in 6.7 mM DTT in PBS, 1:3 g/v, prior. Samples were subsequently thawed and centrifuged, and supernatant transferred into fresh tubes prior to freeze drying. Samples were reconstituted in 0.1% formic acid prior to injection by HPLC-MS/MS. Plasma and brain psilocin was quantified against matrix matched calibration curves, respectively, and quality controls were additionally performed. Standards and quality controls were extracted in the same manner as test samples.

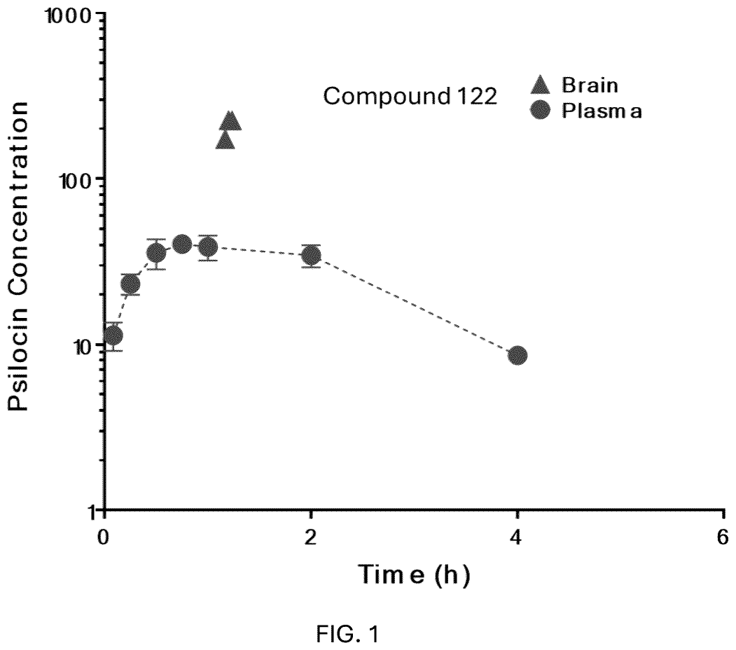

Data analysis: pharmacokinetics parameters were estimated using non-compartmental analysis in WinNon-Lin (Certara, Phoenix, Version 8.3.5.340). The PK data of compound 122 are included in FIG. 1, which shows that Compound 122 acts as a psilocin prodrug after a subcutaneous dose of 1.3 mg/kg, delivering psilocin to the plasma and brain in the rat with a Tₘₐₓ of approximately 1 hour.

### Example 16. Synthesis of Compound 222 and Fumarate Salt of Compound 222

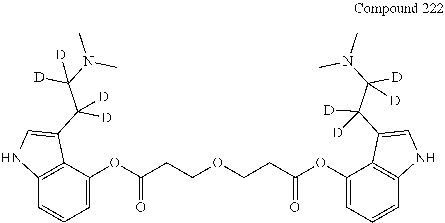 [SMILES: [2H]C([2H])(c1c[nH]c2cccc(OC(=O)CCOCCC(=O)Oc3cccc4[nH]cc(C([2H])([2H])C([2H])([2H])N(C)C)c34)c12)C([2H])([2H])N(C)C]

### Step 1: Synthesis of Tetradueterated Psilocin

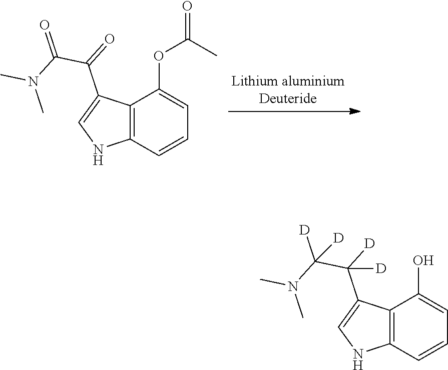 [SMILES: CC(=O)Oc1cccc2[nH]cc(C(=O)C(=O)N(C)C)c12 ; [2H]C([2H])(c1c[nH]c2cccc(O)c12)C([2H])([2H])N(C)C]

A reaction flask (250 mL) was purged with nitrogen. To the reaction flask was charged with 3-(2-(dimethylamino)-2-oxoacetyl)-1H-indol-4-yl acetate (7.40 g, 1 eq, 27.0 mmol) and 2-Methyltetrahydrofuran (81.4 mL). The mixture was stirred at ambient temperatures (Tp=22 C, RPM=500) to dissolve 3-(2-(dimethylamino)-2-oxoacetyl)-1H-indol-4-yl acetate.

Lithium aluminum deuteride (4.25 g, 3.75 eq, 101 mmol) was weighed out into 8 separate vials (Vial 1=0.25 g; Vial 2-6=0.5 g; Vial 7-8=0.75 g). The lithium aluminum deuteride from each vial was added into the reaction flask. The solid charge part (for adding the lithium aluminum deuteride) was rinsed with 2-Methyltetrahydrofuran (7.40 mL) and the rinse was added the reaction flask.

The reaction mixture was heated to reflux for no less than (NLT) four hours. After the reaction is completed, the reaction was cooled down in an ice-bath. A water-THE mixture (1.82 mL water and 6.48 THF) was added dropwise to the reaction mixture to quench the reaction. The reaction flask was lifted from the ice bath and the mixture was stirred at ambient temperatures for no less than 30 mins. Silica gel (7.40 g, 1 eq, 27.0 mmol) and sodium sulphate (7.40 g, 1 eq, 27.0 mmol) were charged in one portion to the reaction flask and allowed to stir at ambient temperatures for NLT 30 mins.

The liquid contents of the reaction flask were decanted to filter 1 equipped with nitrogen cone. The reaction flask was washed with THF (74.0 mL) by adding the THF into the flask and stirring the THF for NLT 5 mins. After stirring, the liquid contents of the reaction flask were decanted into filter 1. The reaction flask was washed four more times with THF (74.0 mL×4) and the resulting liquid was decanted into filter 1. The combined liquid contents in filter 1 was filtered and the filtrate was collected into receiver 1. The solids and remaining wash on filter 1 were transferred back to the reaction flask, rinsed with THF (7.5 mL, 1 vol) and allowed to stir for NLT 10 mins. The resulting mixture in the reaction flask was transferred into filter 2. The reaction flask was washed with 74.0 mL THF and the resulting content in the flask was transferred to filter 2. The filtrate from filter 2 was collected into receiver 1. The combined filtrate in receiver 1 was filtered through a sinter filter packed with a celite pad of 1 cm. The filtrate was collected into a 250 ml conical flask and stirred overnight under nitrogen. After stirring, the filtrate in the conical flask was concentrated to dryness under reduced pressure to generate a first batch of crude tetradeuterated psilocin. The remained solid in the sinter filter was washed with DCM: MeOH (10%, 74 mL×3). The wash was filtered, and the filtrate was collected and concentrated to dryness under pressure to a second batch of crude tetradeuterated psilocin.

The crude tetradeuterated psilocin was recrystallized in isopropyl acetate to generate purified tetradeuterated psilocin (2.350 g) with chemical purity of 99.34 area % (HPLC), NMR assay purity of 96.32 w/w % and residual solvent of 1.17 w/w % (qNMR).

### Step 2: Synthesis of Compound 222

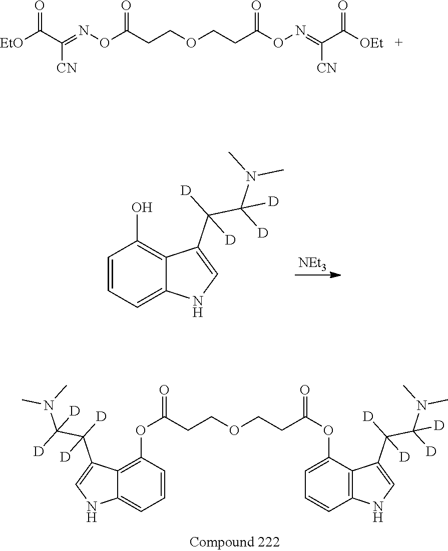

To a 100 mL round bottom flask equipped with a magnetic stirrer bard and a T-piece adapter for a flow of nitrogen was charged with 3-(2-(dimethylamino)ethyl-1,1,2,2-d4)-1H-indol-4-ol (2.30 g, 2 Eq, 11.0 mmol) and ethyl acetate (9.57 mL). The resulting mixture in the flask was stirred at room temperature. The reaction flask was inerted with nitrogen. Diethyl (2E,13E)-2,14-dicyano-5,11-dioxo-4,8,12-trioxa-3,13-diazapentadeca-2,13-dienedioate (2.70 g, 84% Wt, 1 eq, 5.52 mmol) dissolved in 12.7 mL ethyl acetate was added dropwise to the flask. The resulting mixture in the flask was stirred at room temperature for less than 2 hours. Triethylamine (2.28 g, 3.1 mL, 4.09 eq, 22.6 mmol) was charged to the flask and the resulting mixture in the flask was stirred for no less than 30 minutes. Water (11.5 mL) and ethyl acetate (11.5 mL) was charged to the reaction flask and the resulting mixture in the flask was stirred for no less than 15 mins. The organic layer of the resulting solution was separated through extraction and washed with water (11.5 mL×3). The resulting organic layer was concentrated to dryness under vacuum to afford crude compound 222 (2.24 g) with a purity of 95.2 area % (HPLC) and 74.05 w/w % (qH-NMR).

### Step 2: Synthesis of Fumarate Salt of Compound 222

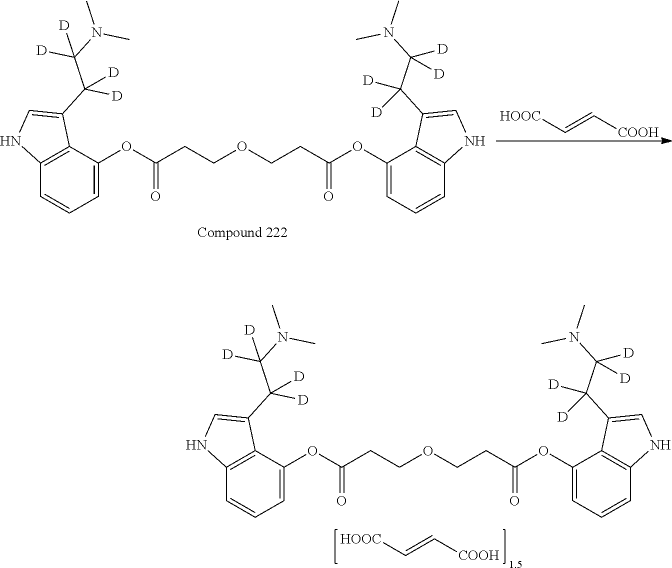

To a round bottom flask (25 mL) was charged with bis(3-(2-(dimethylamino)ethyl-1,1,2,2-d4)-1H-indol-4-yl) 3,3′-oxydipropionate (1.66 g, 1 eq, 3.06 mmol), isopropanol (IPA, 8.72 mL), water (2.17 mL) and fumaric acid (710 mg, 2 eq, 6.12 mmol). The resulting mixture in the flask was stirred at room temperature overnight. After stirring, the mixture in the flask was filtered over a sinter connected to a cone of nitrogen. The flask was rinsed twice with 2:1 mixture of IPA: water (1.66 mL×2) and the rinse was filtered with sinter connected to a cone of nitrogen.

The solid obtained from the filtration, along with isopropanol (7.67 mL) and water (3.80 mL), were added to a second round bottom flask (25 mL). The mixture in the second flask was heated to 80° C. and filtered at 80° C. and the filtrate was collected into a third flask. The mixture in the third flask was cooled to room temperature and stirred at room temperature for no less than 4 hours. The stirring was stopped and the mixture in the third flask was filtered. The third flask was rinsed twice with 2:1 mixture of IPA: water (2.3 mL×2) and the rinse was filtered. The filtration afforded fumarate salt of Compound 222 as solid (1.59 g, 72% yield) with a purity of 98.69 area % (HPLC). ¹H NMR (500 MHz, DMSO) δ 11.12 (d, J=1.4 Hz, 2H), 7.22 (d, J=8.2 Hz, 2H), 7.16 (d, J=2.3 Hz, 2H), 6.99 (t, J=7.9 Hz, 2H), 6.65 (d, J=7.6 Hz, 2H), 6.51 (s, 3H), 3.83 (t, J=6.3 Hz, 4H), 2.99 (t, J=6.3 Hz, 4H), 2.46 (s, 12H).

1. A compound of formula (I), 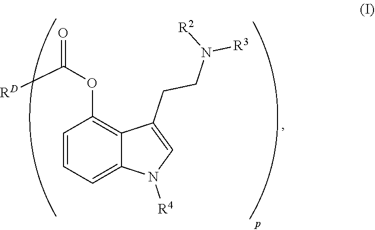
or a pharmaceutically acceptable salt thereof or deuterated form thereof,
wherein:
R² and R³ are independently alkyl;
R⁴ is H or C(═O)Oalkyl;
p is 2 or 3;
R^{D} is a divalent or trivalent group selected from alkylene, alkenylene, alkylene-O-alkylene, —(CH₂)ₙ—(OCH₂CH₂)ₘ—, cycloalkylene, or arylene, or 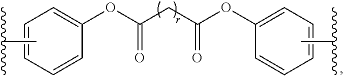
each of which is optionally substituted with 1-4 groups selected from halogen, OH, Oalkyl, alkyl, NH₂, NH(alkyl), N(alkyl)₂, C(═O)OH, C(═O)Oalkyl, OC(═O)alkyl, or C(═O)alkyl;
n is 1, 2, 3, 4, 5, 6, 7 or 8;
m is 1, 2, 3, 4, 5, 6, 7 or 8;
r is 1, 2 or 3;
provided that when R² and R³ are each non-deuterated alkyl, then R^{D} is not CH₂, (CH₂)₂, (CH₂)₃, (CH₂)₄, (CH₂)₅, (CH₂)₈, 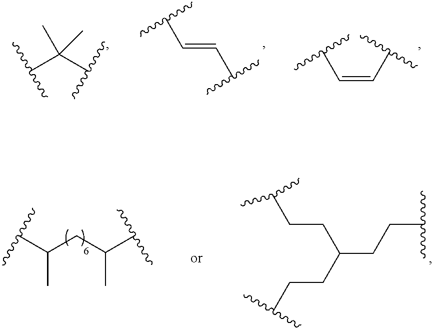
and
provided that when R² and R³ are each CD₃ then R^{D} is not (CH₂)₃.

2. The compound of claim 1, wherein R^{D} is a divalent or trivalent group selected from C₁₋₁₂ alkylene, C₁₋₆alkylene-O—C₁₋₆alkylene, —(CH₂)ₙ—(OCH₂CH₂)ₘ—, C₃₋₈cycloalkylene, arylene or 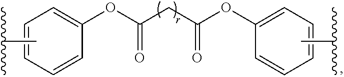
each of which is optionally substituted with 1-4 groups selected from with C₁₋₃ alkyl, OH, OCH₃, NH₂, COOH, or C(═O)alkyl.

3. The compound of claim 1, wherein R^{D} is a divalent or trivalent group selected from alkylene-O-alkylene, —(CH₂)ₙ—(OCH₂CH₂)ₘ—, cycloalkylene, heterocyclylene, arylene, heteroarylene or 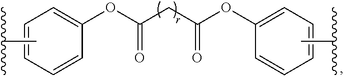
each of which is optionally substituted with OH, OCH₃, NH₂, COOH, cycloalkyl, or C(═O)alkyl.

4. The compound of claim 1, wherein R^{D} is a divalent or trivalent group selected from C₁₋₆alkylene-O—C₁₋₆alkylene, —(CH₂)ₙ—(OCH₂CH₂)ₘ—, C₃₋₈cycloalkylene, 3-8 membered heterocyclylene, arylene, heteroarylene or 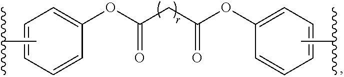
each of which is optionally substituted with OH, OCH₃, NH₂, COOH, cycloalkyl, or C(═O)alkyl.

5. The compound of claim 1, wherein the compound is a compound of formula (Ia) 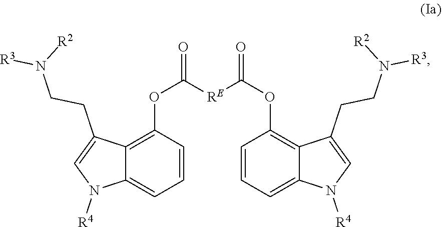
or a pharmaceutically acceptable salt or deuterated thereof,
wherein:
each R² and R³ are independently alkyl;
each R⁴ is independently H or C(═O)Oalkyl;
R^{E} is a divalent group selected from alkylene, cycloalkylene, arylene, alkylene-O-alkylene, —(CH₂)ₙ—(OCH₂CH₂)ₘ— or 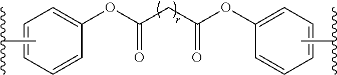
each of which is optionally substituted with one or more groups selected from halogen, alkyl, Oalkyl, NH₂, NHC₁₋₆alkyl, NC(₁₋₆alkyl)₂, COOH, cycloalkyl, or C(═O)Oalkyl;
n is 1, 2, or 3;
m is 1, 2 or 3; and
r is 1, 2 or 3.

6. The compound of claim 5, wherein R^{E} is a divalent group selected from C₁₋₁₂ alkylene, C₃₋₈cycloalkylene, arylene, C₁₋₆alkylene-O—C₁₋₆alkylene, —(CH₂)ₙ—(OCH₂CH₂)ₘ— or 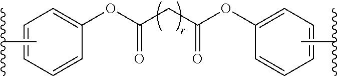
each of which is optionally substituted with one or more groups selected from halogen, C₁₋₃alkyl, Oalkyl, NH₂, NHC₁₋₆alkyl, NC(₁₋₆alkyl)₂, COOH, C₃₋₈ cycloalkyl, or C(═O)Oalkyl.

7. The compound of claim 5, wherein the alkylene group is linear or branched.

8. The compound of claim 6, wherein the alkylene group is branched.

9. The compound of claim 5, wherein R^{E} is 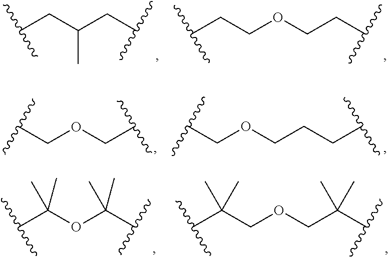
—(CH₂)₂—(OCH₂CH₂)₂—, —(CH₂)₂—(OCH₂CH₂)₃—,

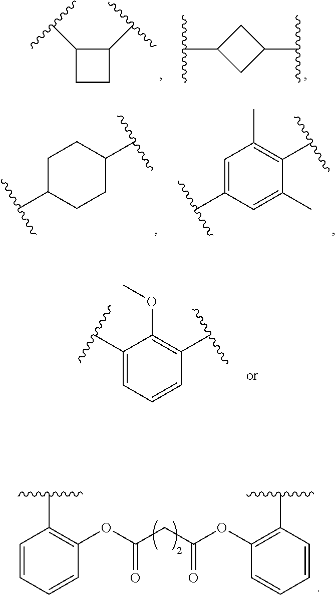

10. The compound of claim 1, wherein the compound is a compound of formula (Ib) 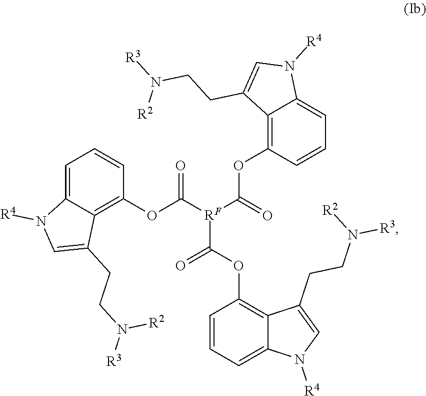
or a pharmaceutically acceptable salt or deuterated form thereof,
wherein:
each R² and R³ are independently alkyl;
each R⁴ is independently H or C(═O)Oalkyl; and
R^{F} is a trivalent cycloalkylene or alkylene group.

11. The compound of claim 10, wherein R^{F} is a trivalent C₃₋₈cycloalkylene.

12. The compound of claim 10, wherein R^{F} is

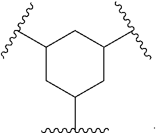 [SMILES: *C1CC(*)CC(*)C1]

13. A compound of formula (IIa), 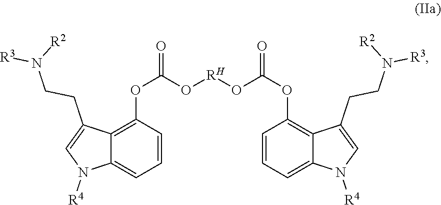
or a pharmaceutically acceptable salt or deuterated form thereof,
wherein:
each R² and R³ are independently alkyl;
each R⁴ is independently H or C(═O)Oalkyl;
R^{H} is —(CH₂)ₙ—(OCH₂CH₂)ₘ— or alkylene;
n is 1, 2, 3, 4, 5, 6, 7, 8, 9, 10, 11 or 12,
m is 1, 2 or 3; and
provided that when R² and R³ are each CH₃, R^{H} is not —(CH₂)₂— or —(CH₂)₃—.

14. The compound of claim 13, wherein,
n is 1, 2, 3, 4, 5, 6, 7, or 8, and
m is 1, 2 or 3.

15. The compound of claim 13, wherein R^{H} is —(CH₂)₅—, —(CH₂)₈— or —(CH₂)₂—(OCH₂CH₂)₂.

16. A compound of formula (III) 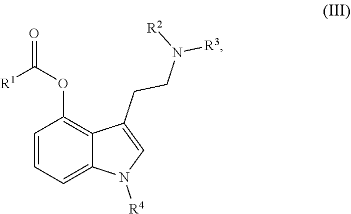
or a pharmaceutically acceptable salt thereof or deuterated form thereof,
wherein:
R² and R³ are independently alkyl;
R⁴ is H or C(═O)Oalkyl;
R¹ is alkylene-C(═O)OH wherein the alkylene is optionally substituted with OH, C(═O)OH, —OC(═O)alkyl or NH₂, alkenylene-C(═O)OH wherein the alkenylene is optionally substituted with C(═O)OH, cycloalkylene-C(═O)OH wherein the cycloalkylene is optionally substituted with C(═O)OH, arylene-C(═O)OH wherein the arylene is optionally substituted with alkyl, Oalkyl or C(═O)OH, —(CH₂CH₂O)ₘ—(CH₂CH₂)ₙ—C(═O)OH, alkylene-O-alkylene-C(═O)OH, arylene-O(C═O)-alkylene-C(═O)O-arylene-C(═O)OH, O-alkylene-OH wherein the alkylene is optionally substituted with OH, or —(OCH₂CH₂)ₘ—OH;
m is 1, 2, or 3;
n is 1, 2, or 3; and
provided that when R² and R³ are each alkyl, then R¹ is not (CH₂)₂C(═O)OH, (CH₂)₃C(═O)OH, —CH═CHC(═O)OH or

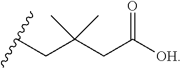 [SMILES: *CC(C)(C)CC(=O)O]

17. The compound of claim 16, wherein R¹ is C₁₋₁₀alkylene-C(═O)OH wherein the alkylene is optionally substituted with OH, C(═O)OH, —OC(═O)alkyl or NH₂, C₁₋₆alkenylene-C(═O)OH wherein the alkenylene is optionally substituted with C(═O)OH, C₃₋₆cycloalkylene-C(═O)OH wherein the cycloalkylene is optionally substituted with C(═O)OH, arylene-C(═O)OH wherein the arylene is optionally substituted with alkyl, Oalkyl or C(═O)OH, —(CH₂CH₂O)ₘ—(CH₂CH₂)ₙ—C(═O)OH, C₁₋₆alkylene-O—C₁₋₆alkylene-C(═O)OH, arylene-O(C═O)—C₁₋₆alkylene-C(═O)O-arylene-C(═O)OH, O—C₁₋₁₀alkylene-OH wherein the alkylene is optionally substituted with OH, or —(OCH₂CH₂)ₘ—OH.

18. The compound of claim 16, wherein R¹ is

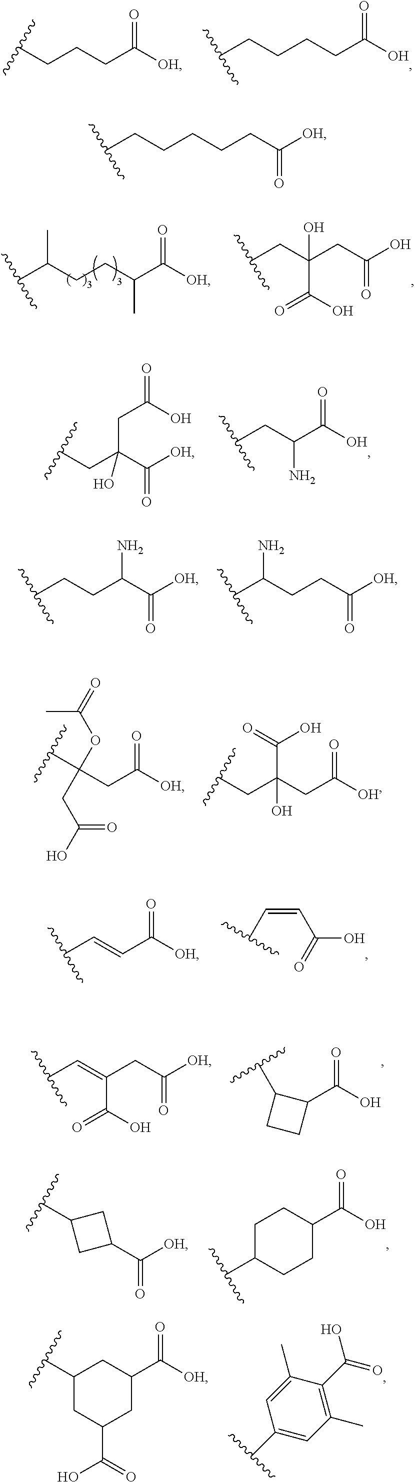

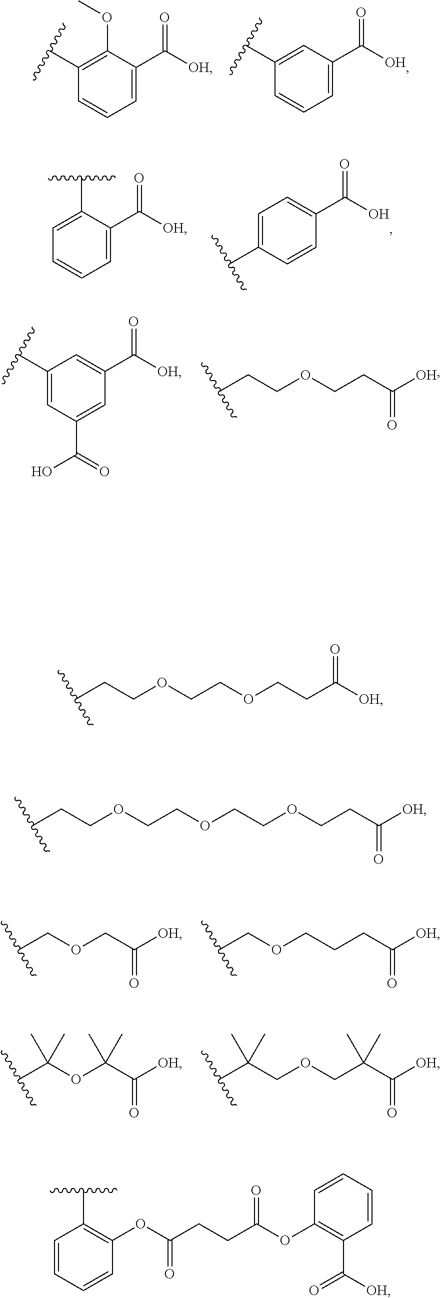

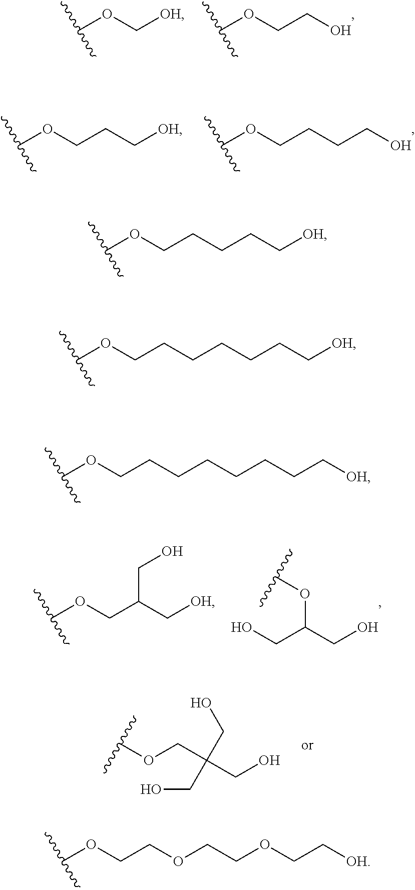

19. The compound of claim 16, wherein R¹ is —(CH₂CH₂O)ₘ—(CH₂CH₂)ₙ—C(═O)OH.

20. The compound of claim 16, wherein R¹ is

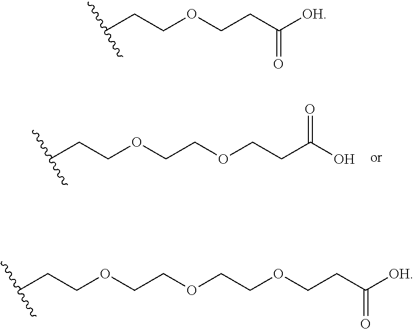 [SMILES: *CCOCCC(=O)O ; *CCOCCOCCC(=O)O ; "or" ; *CCOCCOCCOCCC(=O)O]

21. The compound of claim 16, wherein R¹ is

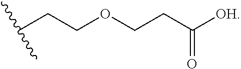 [SMILES: *CCOCCC(=O)O]

22. The compound of claim 16, wherein R² and R³ are independently C₁₋₆ alkyl.

23. The compound of claim 16, wherein R² and R³ are —CH₃.

24. The compound of claim 16, wherein R⁴ is H or C(═O)OC₁₋₆ alkyl.

25. The compound of claim 16, wherein R⁴ is H or C(═O)OCH₃.

26. The compound of claim 16, wherein R⁴ is H.

27. The compound of claim 16, wherein R² and R³ are independently CH₃, and R⁴ is H.

28-32. (canceled)

33. A method of treating a disease comprising subcutaneously administrating a pharmaceutical composition comprising a compound of formula (I), or formula (IIa), formula (III) or a pharmaceutically acceptable salt thereof, or a deuterated form thereof, 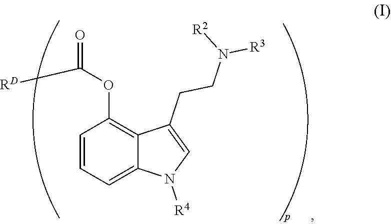
or a pharmaceutically acceptable salt or deuterated form thereof,
wherein:
R² and R³ are independently alkyl;
R⁴ is H or C(═O)Oalkyl;
p is 2 or 3;
R^{D} is a divalent or trivalent group selected from alkyl, alkenyl, alkylene-O-alkylene, —(CH₂)ₙ—(OCH₂CH₂)ₘ—, cycloalkyl, aryl or 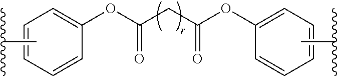
each of which is optionally substituted with OH, Oalkyl, NH₂, NH(alkyl), N(alkyl)₂, COOH, C(═O)Oalkyl, COOH, cycloalkyl or C(═O)alkyl;
n is 1, 2, 3, 4, 5, 6, 7 or 8; m is 1, 2 or 3; and
r is 1, 2, 3;
or 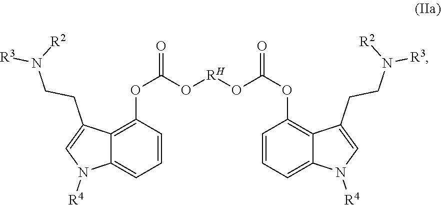
or a pharmaceutically acceptable salt or deuterated form thereof,
wherein:
each R² and R³ are independently alkyl;
each R⁴ is independently H or C(═O)Oalkyl;
R^{H} is —(CH₂)ₙ—(OCH₂CH₂)ₘ— or alkylene;
n is 1, 2, or 3, and
m is 1, 2 or 3;
or 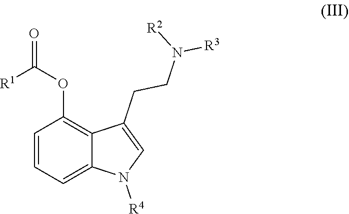
or a pharmaceutically acceptable salt thereof or deuterated form thereof, wherein:
R² and R³ are independently alkyl;
R⁴ is H or C(═O)Oalkyl;
R¹ is alkylene-C(═O)OH wherein the alkylene is optionally substituted with OH, C(═O)OH, —OC(═O)alkyl or NH₂, alkenylene-C(═O)OH wherein the alkenylene is optionally substituted with C(═O)OH, cycloalkylene-C(═O)OH wherein the cycloalkylene is optionally substituted with C(═O)OH, arylene-C(═O)OH wherein the arylene is optionally substituted with alkyl, Oalkyl or C(═O)OH, —(CH₂CH₂O)ₘ—(CH₂CH₂)ₙ—C(═O)OH, alkylene-O-alkylene-C(═O)OH, arylene-O(C═O)-alkylene-C(═O)O-arylene-C(═O)OH, O-alkylene-OH wherein the alkylene is optionally substituted with OH, or —(OCH₂CH₂)ₘ—OH;
m is 1, 2, 3,
n is 1, 2, 3,
provided that when provided that when R² and R³ are each alkyl, then R¹ is not (CH₂)₂C(═O)OH, (CH₂)₃C(═O)OH, —CH═CHC(═O)OH or 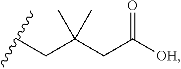 [SMILES: *CC(C)(C)CC(=O)O]
wherein the disease is selected from anxiety disorder, attention deficit hyperactivity disorder (ADHD), depression (including treatment resistant depression), cluster headache, diminished drive, burn-out, bore-out, migraine, Parkinson's disease, schizophrenia, an eating disorder (including anorexia nervosa), psychotic disorder, schizophrenia, schizophreniform disorder, schizoaffective disorder, bipolar I disorder, bipolar II disorder, major depressive disorder, psychotic depression, delusional disorders, shared psychotic disorder, Shared paranoia disorder, brief psychotic disorder, paranoid personality disorder, schizoid personality disorder, schizotypal personality disorder, anxiety disorder, social anxiety disorder, substance-induced anxiety disorder, selective mutism, panic disorder, panic attacks, agoraphobia, attention deficit syndrome, posttraumatic stress disorder (PTSD), premenstrual dysphoric disorder (PMDD), and premenstrual syndrome (PMS).

34-35. (canceled)
